SEQUENCE LISTING (58 sequences)

SEQ ID NO: 1 - DNA, 1167 bp, CANDIDATUS LIBERIBACTER ASIATICUS
gcgcgtatgcaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 2 - DNA, 1158 bp, CANDIDATUS LIBERIBACTER ASIATICUS
agcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgacggccaaggc

SEQ ID NO: 3 - DNA, 1166 bp, CANDIDATUS LIBERIBACTER ASIATICUS
gcgcgtatgcatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 4 - DNA, 1166 bp, CANDIDATUS LIBERIBACTER ASIATICUS; identical to SEQ ID NO: 3

SEQ ID NO: 5 - DNA, 1166 bp, CANDIDATUS LIBERIBACTER ASIATICUS; identical to SEQ ID NO: 3

SEQ ID NO: 6 - DNA, 1167 bp, CANDIDATUS LIBERIBACTER ASIATICUS; identical to SEQ ID NO: 1

SEQ ID NO: 7 - DNA, 1166 bp, CANDIDATUS LIBERIBACTER ASIATICUS; identical to SEQ ID NO: 3

SEQ ID NO: 8 - DNA, 1163 bp, CANDIDATUS LIBERIBACTER ASIATICUS
gcgcgtatgcatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcga

SEQ ID NO: 9 - DNA, 1167 bp, CANDIDATUS LIBERIBACTER ASIATICUS; identical to SEQ ID NO: 1

SEQ ID NO: 10 - DNA, 1168 bp, CANDIDATUS LIBERIBACTER ASIATICUS
gcgcgtatgcaattacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 11 - DNA, 1168 bp, CANDIDATUS LIBERIBACTER ASIATICUS
gcgcgtatgccaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 12 - DNA, 1163 bp, CANDIDATUS LIBERIBACTER ASIATICUS
tatgccaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 13 - DNA, 1146 bp, CANDIDATUS LIBERIBACTER ASIATICUS
gcgcgtatgcatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaag

SEQ ID NO: 14 - DNA, 1167 bp, CANDIDATUS LIBERIBACTER ASIATICUS; identical to SEQ ID NO: 1

SEQ ID NO: 15 - DNA, 1167 bp, CANDIDATUS LIBERIBACTER ASIATICUS; identical to SEQ ID NO: 1

SEQ ID NO: 16 - DNA, 1167 bp, CANDIDATUS LIBERIBACTER ASIATICUS; identical to SEQ ID NO: 1

SEQ ID NO: 17 - DNA, 1167 bp, CANDIDATUS LIBERIBACTER ASIATICUS; identical to SEQ ID NO: 1

SEQ ID NO: 18 - DNA, 1167 bp, CANDIDATUS LIBERIBACTER ASIATICUS
gcgcgtatgcaatacgagcggcagacgggtgagtaacgcgtaggaatctacctttttctacaggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 19 - DNA, 1166 bp, CANDIDATUS LIBERIBACTER ASIATICUS; identical to SEQ ID NO: 3

SEQ ID NO: 20 - DNA, 1167 bp, CANDIDATUS LIBERIBACTER ASIATICUS
gcgcgtatgcaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactacctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 21 - DNA, 1167 bp, CANDIDATUS LIBERIBACTER ASIATICUS
gcgcgtatgcaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcacaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 22 - DNA, 1167 bp, CANDIDATUS LIBERIBACTER ASIATICUS; identical to SEQ ID NO: 1

SEQ ID NO: 23 - DNA, 1168 bp, CANDIDATUS LIBERIBACTER ASIATICUS; identical to SEQ ID NO: 11

SEQ ID NO: 24 - DNA, 1167 bp, CANDIDATUS LIBERIBACTER ASIATICUS
gcgcgtatgccatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 25 - DNA, 1166 bp, CANDIDATUS LIBERIBACTER ASIATICUS; identical to SEQ ID NO: 3

SEQ ID NO: 26 - DNA, 1166 bp, CANDIDATUS LIBERIBACTER ASIATICUS; identical to SEQ ID NO: 3

SEQ ID NO: 27 - DNA, 1168 bp, CANDIDATUS LIBERIBACTER ASIATICUS; identical to SEQ ID NO: 11

SEQ ID NO: 28 - DNA, 1167 bp, CANDIDATUS LIBERIBACTER ASIATICUS; identical to SEQ ID NO: 1

SEQ ID NO: 29 - DNA, 1167 bp, CANDIDATUS LIBERIBACTER ASIATICUS; identical to SEQ ID NO: 1

SEQ ID NO: 30 - DNA, 1507 bp, CANDIDATUS LIBERIBACTER ASIATICUS
atgagagtttgatcctggctcagaacgaacgctggcggcaggcctaacacatgcaagtcgagcgcgtatgcgaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggcggagctaatccccaaaagccatctcagttcggattgcactctgcaactcgagtgcatgaagtcggaatcgctagtaatcgcggatcagcatgccgcggtgaatacgttctcgggccttgtacacaccgcccgtcacaccatgggagttggttttgcctgaagacggtgtgctaaccgcaaggaggcagccggccacggtagggtcagcgactggggtgaagtcgtaacaaggtagccgtaggggaacctgcggctggatcacctcctttctaaggaag

SEQ ID NO: 31 - DNA, 23 bp, LIBERIBACTER
gcgcgtatgcaatacgagcggca

SEQ ID NO: 32 - DNA, 22 bp, LIBERIBACTER
gcctcgcgacttcgcaacccat

SEQ ID NO: 33 - DNA, 25 bp, LIBERIBACTER
cttatcaccggcagtccctataaag

SEQ ID NO: 34 - DNA, 22 bp, LIBERIBACTER
cagctcgtgtcgtgagatgttg

SEQ ID NO: 35 - DNA, 27 bp, LIBERIBACTER
ttgtttcgatatccgaatctatgcggg

SEQ ID NO: 36 - DNA, 30 bp, LIBERIBACTER
atatttgattctcgtgcaatttgtagcaac

SEQ ID NO: 37 - DNA, 21 bp, LIBERIBACTER
tctaaagcaggacgtttgtgt

SEQ ID NO: 38 - DNA, 24 bp, LIBERIBACTER
tgagtatatttgattctcgtgcaa

SEQ ID NO: 39 - DNA, 23 bp, L. CRESCENS, PGI
acttgccaaaccaaccgttgaag

SEQ ID NO: 40 - DNA, 23 bp, L. CRESCENS, PGI
accaattcttgtgatattcgggc

SEQ ID NO: 41 - DNA, 20 bp, L. CRESCENS, PGI
cgctctcgatgggattggaa

SEQ ID NO: 42 - DNA, 20 bp, L. CRESCENS, PGI
ctgaggtttctgtccccgtc

SEQ ID NO: 43 - DNA, 25 bp, CA. L. ASIATICUS
ggtttttacctagatgttgggtact

SEQ ID NO: 44 - DNA, 19 bp, CA. L. ASIATICUS
ctcgcaacccattgtaacc

SEQ ID NO: 45 - DNA, 1161 bp, CANDIDATUS LIBERIBACTER ASIATICUS
agcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctaccacacgtgctacaatggtggttaccaattgggttgcgaagtcgcgacggcaagggc

SEQ ID NO: 46 - DNA, 1165 bp, CANDIDATUS LIBERIBACTER ASIATICUS
gcgcgtatgcatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgctgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggctacaatgggtgcgaagtcgcgaggc

SEQ ID NO: 47 - DNA, 1168 bp, CANDIDATUS LIBERIBACTER ASIATICUS
gcgcgtatgcatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgctgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacaccacgtgctacaatggtggctaccaatgggttgcgaagtcgcgaggc

SEQ ID NO: 48 - DNA, 1167 bp, CANDIDATUS LIBERIBACTER ASIATICUS
gcgcgtatgccaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggtgcgaagtcgcgaggc

SEQ ID NO: 49 - DNA, 1166 bp, CANDIDATUS LIBERIBACTER ASIATICUS
gcgcgtatgcaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaatttgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggtgcgaagtcgcgaggc

SEQ ID NO: 50 - DNA, 1168 bp, CANDIDATUS LIBERIBACTER ASIATICUS
gcgcgtatgcaatacgagcggcagacgggtgagtaacgcgtgggaatctacctttttctacaggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattctcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttaccaatgggttgcgaagtcgcgaggc

SEQ ID NO: 51 - DNA, 1169 bp, CANDIDATUS LIBERIBACTER ASIATICUS
gcgcgtatgcatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctaccacacgtgctaccaatggtggttaccaatgggttgcgaagtcgcgaggc

SEQ ID NO: 52 - DNA, 1166 bp, CANDIDATUS LIBERIBACTER ASIATICUS
gcgcgtatgcaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactacctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggtgcgaagtcgcgaggc

SEQ ID NO: 53 - DNA, 1166 bp, CANDIDATUS LIBERIBACTER ASIATICUS
gcgcgtatgcaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcacaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggtgcgaagtcgcgaggc

SEQ ID NO: 54 - DNA, 1168 bp, CANDIDATUS LIBERIBACTER ASIATICUS
gcgcgtatgcaatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattccgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 55 - DNA, 1168 bp, CANDIDATUS LIBERIBACTER ASIATICUS
gcgcgtatgccatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattccgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 56 - DNA, 1167 bp, CANDIDATUS LIBERIBACTER ASIATICUS
gcgcgtatgcatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaactgcctttaatactggttgtctagagtttaggagaggtgagtggaattccgagtgtagaggtgaaattccgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcactccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 57 - DNA, 1169 bp, CANDIDATUS LIBERIBACTER ASIATICUS
gcgcgtatgcatacgagcggcagacgggtgagtaacgcgtaggaatctacctttttctacgggataacgcatggaaacgtgtgctaataccgtatacgccctattgggggaaagattttattggagagagatgagcctgcgttggattagctagttggtagggtaagagcctaccaaggctacgatctatagctggtctgagaggacgatcagccacactgggactgagacacggcccagactcctacgggaggcagcagtggggaatattggacaatgggggcaaccctgatccagccatgccgcgtgagtgaagaaggccttagggttgtaaagctctttcgccggagaagataatgacggtattcggagaagaagccccggctaacttcgtgccagcagccgcggtaatacgaagggggcgagcgttgttcggaataactgggcgtaaagggcgcgtaggcgggcgattaagttagaggtgaaatcccagggctcaaccttggaaactgccctttaatactggttgtctagagtttaggagaggtgagtggaatttccgagtgtagaggtgaaattcgtagatattcggaggaacaccggtggcgaaggcggctcactggcctgatactgacgctgaggcgcgaaagcgtggggagcaaacaggattagataccctggtagtccacgccgtaaacgatgagtgctagctgttgggtggtttaccattcagtggcgcagctaacgcattaagcattccgcctggggagtacggtcgcaagattaaaactcaaaggaattgacgggggcccgcacaagcggtggagcatgtggtttaattcgatgcaacgcgcagaaccttaccagcccttgacatgtataggacgatatcagagatggtattttcttttcggagacctttacacaggtgctgcatggctgtcgtcagctcgtgtcgtgagatgttgggttaagtcccgcaacgagcgcaacccctgcctctagttgccatcaagtttaggtttttacctagatgttgggtactttatagggactgccggtgataagccggaggaaggtggggatgacgtcaagtcctcatggcccttatgggctgggctacacacgtgctacaatggtggttacaatgggttgcgaagtcgcgaggc

SEQ ID NO: 58 - DNA, 1167 bp, CANDIDATUS LIBERIBACTER ASIATICUS; identical to SEQ ID NO: 48

(19) United States
(12) Patent Application Publication
(10) Pub. No.: US 2021/0403860 A1
(43) Pub. Date: Dec. 30, 2021

(54) HOST-FREE 'CANDIDATUS LIBERIBACTER ASIATICUS' CULTURE

(71) Applicant: WASHINGTON STATE UNIVERSITY, Pullman, WA (US)

(72) Inventors: David Roger GANG, Moscow, ID (US); Haluk BEYENAL, Pullman, WA (US); Anders OMSLAND, Moscow, ID (US); Elham ATTARAN, Pullman, WA (US); Phuc HA, Richland, WA (US); Banafsheh MOLKI, Pullman, WA (US); Douglas CALL, Pullman, WA (US)

(21) Appl. No.: 17/293,696

(22) PCT Filed: Nov. 14, 2019
(86) PCT No.: PCT/US2019/061483

§ 371 Date: May 13, 2021

Related U.S. Application Data
(60) Provisional application No. 62/767,053, filed on Nov. 14, 2018.
(60) Provisional application No. 62/813,495, filed on Mar. 4, 2019.
(60) Provisional application No. 62/907,436, filed on Sep. 27, 2019.

(51) Int. Cl. C12N 1/20; C12N 1/38

(52) U.S. Cl. CPC C12N 1/20; C12N 1/38

(57) ABSTRACT

The invention relates to compositions comprising Candidatus Liberibacter. The invention includes growth mediums, microbial cultures, methods, assays, and kits related to the compositions comprising Candidatus Liberibacter.

### CROSS-REFERENCE TO RELATED APPLICATIONS

This application claims the benefit under 35 USC § 119(e) of U.S. Provisional Application Ser. No. 62/767,053, filed on Nov. 14, 2018, of U.S. Provisional Application Ser. No. 62/813,495, filed on Mar. 4, 2019, and of U.S. Provisional Application Ser. No. 62/907,436, filed on Sep. 27, 2019. The entire disclosures of each of the three claimed U.S. Provisional Applications are incorporated herein by reference.

### GOVERNMENT RIGHTS

This invention was made with government support under grant no. 2016-70016-24824 awarded by United States Department of Agriculture through the National Institute of Food & Agriculture. The government has certain rights in the invention.

### TECHNICAL FIELD

The invention relates to compositions comprising Candidatus Liberibacter. The invention includes growth mediums, microbial cultures, methods, assays, and kits related to the compositions comprising Candidatus Liberibacter.

### BACKGROUND AND SUMMARY OF THE INVENTION

Huanglongbing (HLB) is a destructive disease primarily affecting citrus trees. The disease, also known as citrus greening disease, is widespread all over the world and currently threatens the existence of the citrus industry. This disease is one of the most important challenges to the citrus industry, costing at least S3.6 billion annually in the United States.

Diagnostic tests have consistently identified the association of ‘Candidatus Liberibacter sp.’ with citrus trees carrying HLB symptoms. ‘Candidatus Liberibacter sp.’ are characterized as phloem-restricted alpha-proteobacteria that have not yet been able to culture independently from the host. Currently, there are three species of ‘Candidatus Liberibacter sp.’ that have been described as the cause of HLB in different countries and climates: ‘Candidatus Liberibacter asiaticus’, ‘Candidatus Liberibacter americanus’ and ‘Candidatus Liberibacter africanus’. Among them, ‘Candidatus Liberibacter asiaticus’ (‘C. L. asiaticus’) is the main cause of HLB in America's citrus industry.

The isolation of ‘Candidatus Liberibacter sp.’ is critical to determining whether the species are actually the causes of HLB or are only parts of the syndrome. If ‘Candidatus Liberibacter sp.’ are the main reason for the disease, having a host-free culture is vital to developing long-term strategies for curing this devastating HLB. It is also to be noted that ‘Candidatus Liberibacter solanacearum,’ (Ca. L. solanacearum) has a genome sequence similar to that of “Ca. L. asiaticus,” and it also carries an ATP translocase like that of ‘Ca. L. asiaticus’. This suggests that the pathogens, having similar effects on its hosts and vectors, would necessarily imply that host-free cultures of a Ca. L. solanacearum pathogen would also be beneficial.

Previous reports about the successful development of media or culturing conditions for ‘C. L. asiaticus’ in particular, have not been verified mostly because cultivation could not be independently repeated or ‘C. L. asiaticus’ disappeared after several transfers. ‘C. L. asiaticus’ is a phloem-restricted pathogen which also can grow in the hemolymph of the Asian citrus psyllid (ACP), its primary insect carrier. The bacterium resists in vitro cultivation, even though it multiplies actively in the phloem sap of citrus plants and in the hemolymph of ACPs. This suggests that the phloem sap of citrus plants and the hemolymph of ACPs harbor the unique nutrients and/or conditions that are essential for its growth. These unique nutrients and/or conditions might also come from other microorganisms present in the hosts.

Accordingly, a need exists for compositions of a growth medium that supports a host-free culture of species of Candidatus Liberibacter pathogens that includes Candidatus Liberibacter asiaticus, Candidatus Liberibacter americanus, Candidatus Liberibacter africanus, and Candidatus Liberibacter solanacearum.

Moreover, Candidatus Liberibacter asiaticus has a relatively small genome [˜1.23 Mb] that appears to lack genes for many essential enzymes and other proteins. This reduced genome is consistent with the limited success with growing “Ca. L. asiaticus” in monoculture. Earlier reports of successful axenic culture have not been independently verified. It has been reported that successful culturing “Ca. L. asiaticus” as a co-culture with an actinobacteria species based on conventional polymerase chain reaction (PCR) assays (presence/absence). Attempts to separate these two bacteria were not successful, suggesting that “Ca. L. asiaticus” growth is dependent on metabolites or other factors produced by the actinobacteria.

Adding citrus vein extract to various media including Liber A medium and observed “Ca. L. asiaticus” colonies on agar plates that were subsequently confirmed using real-time PCR (RT-PCR). These colonies did not survive more than 4-5 serial single-colony transfers. Although insightful, citrus vein extract is not a practical solution for laboratory studies given the limited ability to standardize this inoculum. Moreover, because the colonies were not viable after multiple transfers, this method has limited sustainability. Following this work, citrus juice or citrus pulp has also been added to King's B medium where one was able to detect “Ca. L. asiaticus” within a biofilm structure, but serial propagation failed.

One insight from these earlier investigations is that “Ca. L. asiaticus” may require the presence of other microorganisms to produce essential nutrients that it lacks the ability to biosynthesize. The dependence of “Ca. L. asiaticus” (strain Ishil) on cohabiting “helper bacteria” has been suggested. “Ca. L. asiaticus” is known to propagate in a mixed community within a parasitic insect, the Asian citrus psyllid (ACP), and the addition of “Ca. L. asiaticus” itself can also affect these communities. Others have cultured both Gram-positive and Gram-negative bacteria from ACPs, including Bacillus cereus, Paenibacillus sp., Lysinibacillus sp., Staphylococcus saprophyticus, Streptomyces sp., Enterobacteriales, Enterobacter sp., Pantoea agglomerans, Pseudomonales, Pseudomonas putida, Chryseomonas luteola, Alcaligenes xylosoxidans, and Acromobacter sp. An inverse correlation between the abundance of “Ca. L. asiaticus” and that of a syncytium endosymbiont has been suggested, as well as a direct correlation between the abundance of “Ca. L. asiaticus” and that of Wolbachia. The presence of “Ca. L. asiaticus” may have a direct impact on the gene expression of Wolbachia. However, it is not clear whether the changes in “Ca. L. asiaticus” abundance result from the shifts in microbial community members or vice versa. The dependency and the lack of a monoculture for “Ca. L. asiaticus” make the cause and effect difficult to separate in these systems.

Bacillus spp. including B. subtilis, have multiple antibacterial effects including hydrolytic enzymes (e.g. Ytnp) and surfactin that could inhibit “Ca. L. asiaticus” growth. Under specific growth conditions including application of antibiotics at sub-inhibitory conditions the expression of lactonase proteins in Bacillus spp. (B. subtilis) will be induced. Also, constructed strains of Bacillus spp induce the expression of hydrolytic enzymes; for example, in AcodY strains of B. subtilis, the expression of hydrolytic enzymes is 5 times higher than wild-type B. subtilis which is similar to the application of sub-inhibitory concentrations of streptomycin. The endotherapy of plant diseases using endophytes strains of Bacillus spp especially B. subtilis, as described herein, enable a biocontrol strategy for plant diseases.

Accordingly, a need exists for cultures, methods, assays, and kits that include embodiments in which both i) antibiotics are effective to inhibit growth of the Candidatus Liberibacter inoculum and ii) antibiotics are not effective to inhibit growth of the Candidatus Liberibacter inoculum.

Finally, the ability to activate “Ca. L. asiaticus” DNA replication in the context of natural tissue can be explored. The “Ca. L. asiaticus” genome has been derived via metagenomics-based assembly and metabolic pathway reconstruction based on the genome sequence has been used to predict major metabolic features of “Ca. L. asiaticus”. Similar to other bacterial obligate intracellular parasites including species of the genus Rickettsia and phytoplasmas, the “Ca. L. asiaticus” genome has undergone genome reduction suggesting that the bacterium relies on the host to obtain essential metabolites in order to replicate. “Ca. L. asiaticus” appears to be adapted to the lower oxygen tension of phloem sap (˜7%) and the genome encodes some components necessary for aerobic respiration. However, genes for cytochrome bd (cydAB), a terminal oxidase associated with bacteria specifically adapted to microaerobic environments, do not appear to be encoded by the “Ca. L. asiaticus” genome. Moreover, “Ca. L asiaticus” appears to encode a partial glycolytic pathway in which the gene pgi, encoding glucose-6-phosphate isomerase, is missing. This apparent defect would likely severely reduce the efficiency of “Ca. L. asiaticus” glucose metabolism.

Accordingly, a need exists for assays and kits to more effectively screen variables that impact the potential for “Ca. L. asiaticus” DNA replication, assays and kits can be developed that allow quantification of the absolute load of “Ca. L. asiaticus” DNA within the assay incubated under different physicochemical and nutritional conditions. Conditions that produce an increase in relative DNA content can represent conditions likely to trigger and/or support “Ca. L. asiaticus” replication within leaf tissue. The effects of glucose and oxygen availability can be tested on “Ca. L. asiaticus” replication of DNA in situ.

The following numbered embodiments are contemplated and are non-limiting:
1. A bacterial growth medium, comprising:
a composition, wherein the composition further comprises:
a plurality of nutrients and
a Candidatus Liberibacter inoculum.
2. The bacterial growth medium of clause 1, wherein the composition is configured as a solidified media with an adjusted neutral pH.
3. The bacterial growth medium of clause 1 or clause 2, wherein the plurality of nutrients comprises a first plurality of nutrients and a second plurality of nutrients.
4. The bacterial growth medium of clause 3, wherein the second plurality of nutrients comprises at least one of trace minerals, salts, and vitamins to enhance the composition.
5. The bacterial growth medium of any of clauses 1 to 4, wherein the plurality of nutrients comprises least one of nutrient selected from the group consisting of alpha-ketoglutaric acid, ACE buffer, potassium hydroxide, phosphate buffer, deionized water, and any combination thereof.
6. The bacterial growth medium of any of clauses 1 to 5, wherein the plurality of nutrients comprises one or more trace minerals.
7. The bacterial growth medium of clause 6, wherein the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickel chloride, sodium tungstate, sodium selenite, and any combination thereof.
8. The bacterial growth medium of any of clauses 1 to 7, wherein the plurality of nutrients comprises one or more salts.
9. The bacterial growth medium of clause 8, wherein the salts comprise at least one salt selected from the group consisting of potassium chloride, ammonium chloride, sodium hydrogen phosphate, calcium chloride, magnesium sulfate. and any combination thereof.
10. The bacterial growth medium of any of clauses 1 to 9, wherein the plurality of nutrients comprises one or more vitamins
11. The bacterial growth medium of clause 10, wherein the vitamins comprise at least one vitamin selected from the group consisting of biotin, folic acid, pyridoxine hydrochloride, riboflavin, thiamine hydrochloride, nicotinic acid, DL-calcium pantothenate, vitamin B₁₂, p-aminobenzoic acid, thiocidic acid, and any combination thereof.
12. The bacterial growth medium of any of clauses 1 to 11, wherein the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof.
13. The bacterial growth medium of any of clauses 1 to 11, wherein the Candidatus Liberibacter inoculum is Candidatus Liberibacter asiaticus.
14. The bacterial growth medium of any of clauses 1 to 11, wherein the Candidatus Liberibacter inoculum is Candidatus Liberibacter americanus.
15. The bacterial growth medium of any of clauses 1 to 11, wherein the Candidatus Liberibacter inoculum is Candidatus Liberibacter africanus.
16. The bacterial growth medium of any of clauses 1 to 11, wherein the Candidatus Liberibacter inoculum is Candidatus Liberibacter solanacearum.
17. The bacterial growth medium of any of clauses 1 to 16, wherein the medium is configured to support the growth of the Candidatus Liberibacter inoculum as a culture in a biofilm form.
18. The bacterial growth medium of any of clauses 1 to 17, wherein the composition is configured to have a pH between about 7 and a pH of about 12.
19. The bacterial growth medium of any of clauses 1 to 17, wherein the composition is configured to have a pH between about 7 and a pH of about 11.
20. The bacterial growth medium of any of clauses 1 to 17, wherein the composition is configured to have a pH between about 7 and a pH of about 10.
21. The bacterial growth medium of any of clauses 1 to 17, wherein the composition is configured to have a pH between about 7 and a pH of about 9.
22. The bacterial growth medium of any of clauses 1 to 17, wherein the composition is configured to have a pH between about 7 and a pH of about 8.1.
23. The bacterial growth medium of any of clauses 1 to 17, wherein the composition is configured to have a pH between about 7 and a pH of about 8.
24. The bacterial growth medium of any of clauses 1 to 23, wherein the composition is configured to have an oxygen tension of less than about 30% of air.
25. The bacterial growth medium of any of clauses 1 to 23, wherein the composition is configured to have an oxygen tension of between about 10% of air and about 30% of air.
26. The bacterial growth medium of any of clauses 1 to 23, wherein the composition is configured to have an oxygen tension of between about 15% of air and about 25% of air.
27. The bacterial growth medium of any of clauses 1 to 23, wherein the composition is configured to have an oxygen tension of between about 10% of air and about 20% of air.
28. The bacterial growth medium of any of clauses 1 to 23, wherein the composition is configured to have an oxygen tension of less than about 10% of air.
29. The bacterial growth medium of any of clauses 1 to 23, wherein the composition is configured to have an oxygen tension of between about 5% of air and about 10% of air.
30. The bacterial growth medium of any of clauses 1 to 23, wherein the composition is configured to have an oxygen tension of between about 1% of air and about 10% of air.
31. The bacterial growth medium of any of clauses 1 to 23, wherein the composition is configured to have an oxygen tension of between about 1% of air and about 5% of air.
32. A method of growing a bacterium, said method comprising the steps of:
i) inoculating a composition using a Candidatus Liberibacter bacteria; and
ii) physiochemically adjusting the composition to comprise a pH between 7 and 12 and an oxygen tension of less than about 30% of air of the composition.
33. The method of clause 32, wherein the method further comprises the step of combining a plurality of nutrients with the composition.
34. The method of clause 33, wherein the plurality of nutrients comprises a first plurality of nutrients and a second plurality of nutrients.
35. The method of clause 34, wherein the second plurality of nutrients comprises at least one of trace minerals, salts, and vitamins to enhance the composition.
36. The method of any one of clauses 33 to 35, wherein the plurality of nutrients comprises least one of nutrient selected from the group consisting of alpha-ketoglutaric acid, ACE buffer, potassium hydroxide, phosphate buffer, deionized water, and any combination thereof.
37. The method of any one of clauses 33 to 36, wherein the plurality of nutrients comprises one or more trace minerals.
38. The method of clause 37, wherein the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickel chloride, sodium tungstate, sodium selenite, and any combination thereof.
39. The method of any one of clauses 33 to 38, wherein the plurality of nutrients comprises one or more salts.
40. The method of clause 39, wherein the salts comprise at least one salt selected from the group consisting of potassium chloride, ammonium chloride, sodium hydrogen phosphate, calcium chloride, magnesium sulfate. and any combination thereof.
41. The method of any one of clauses 33 to 39, wherein the plurality of nutrients comprises one or more vitamins.
42. The method of clause 41, wherein the vitamins comprise at least one vitamin selected from the group consisting of biotin, folic acid, pyridoxine hydrochloride, riboflavin, thiamine hydrochloride, nicotinic acid, DL-calcium pantothenate, vitamin B₁₂, p-aminobenzoic acid, thiocidic acid, and any combination thereof.
43. The method of any one of clauses 32 to 42, wherein the method further comprises the step of combining a Candidatus Liberibacter inoculum with the composition.
44. The method of clause 43, wherein the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof.
45. The method of clause 43, wherein the Candidatus Liberibacter inoculum is Candidatus Liberibacter asiaticus.
46. The method of clause 43, wherein the Candidatus Liberibacter inoculum is Candidatus Liberibacter americanus.
47. The method of clause 43, wherein the Candidatus Liberibacter inoculum is Candidatus Liberibacter africanus.
48. The method of clause 43, wherein the Candidatus Liberibacter inoculum is Candidatus Liberibacter solanacearum.
49. The method of any one of clauses 32 to 48, wherein the composition is configured as a solidified media with an adjusted neutral pH.
50. The method of any one of clauses 32 to 49, wherein the Candidatus Liberibacter bacteria is derived from at least one bacteria selected from the group consisting of a leaf and one or more infected psyllids.
51. A kit for a bacterial growth medium, said kit comprising a composition comprising a plurality of nutrients, a Candidatus Liberibacter inoculum, and an instruction for combination of the plurality of nutrients and the Candidatus Liberibacter inoculum.
52. The kit of clause 51, wherein the composition is configured as a solidified media with an adjusted neutral pH.
53. The kit of clause 51 or clause 52, wherein the plurality of nutrients comprises a first plurality of nutrients and a second plurality of nutrients.
54. The kit of clause 53, wherein the second plurality of nutrients comprises at least one of trace minerals, salts, and vitamins to enhance the composition.
55. The kit of any one of clauses 51 to 54, wherein the plurality of nutrients comprises least one of nutrient selected from the group consisting of alpha-ketoglutaric acid, ACE buffer, potassium hydroxide, phosphate buffer, deionized water, and any combination thereof.
56. The kit of any one of clauses 51 to 55, wherein the plurality of nutrients comprises one or more trace minerals.
57. The kit of clause 56, wherein the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickel chloride, sodium tungstate, sodium selenite, and any combination thereof.
58. The kit of any one of clauses 51 to 57, wherein the plurality of nutrients comprises one or more salts.
59. The kit of clause 58, wherein the salts comprise at least one salt selected from the group consisting of potassium chloride, ammonium chloride, sodium hydrogen phosphate, calcium chloride, magnesium sulfate. and any combination thereof.
60. The kit of any one of clauses 51 to 58, wherein the plurality of nutrients comprises one or more vitamins
61. The kit of clause 60, wherein the vitamins comprise at least one vitamin selected from the group consisting of biotin, folic acid, pyridoxine hydrochloride, riboflavin, thiamine hydrochloride, nicotinic acid, DL-calcium pantothenate, vitamin B₁₂, p-aminobenzoic acid, thiocidic acid, and any combination thereof.
62. The kit of any one of clauses 51 to 61, wherein the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof.
63. The kit of any one of clauses 51 to 61, wherein the Candidatus Liberibacter inoculum is Candidatus Liberibacter asiaticus.
64. The kit of any one of clauses 51 to 61, wherein the Candidatus Liberibacter inoculum is Candidatus Liberibacter americanus.
65. The kit of any one of clauses 51 to 61, wherein the Candidatus Liberibacter inoculum is Candidatus Liberibacter africanus.
66. The kit of any one of clauses 51 to 61, wherein the Candidatus Liberibacter inoculum is Candidatus Liberibacter solanacearum.
67. The kit of any one of clauses 51 to 61, wherein the medium is configured to support the growth of the Candidatus Liberibacter inoculum as a culture in a biofilm form.
68. The kit of any one of clauses 51 to 67, wherein the composition is configured to have a pH between about 7 and a pH of about 12.
69. The kit of any one of clauses 51 to 67, wherein the composition is configured to have a pH between about 7 and a pH of about 11.
70. The kit of any one of clauses 51 to 67, wherein the composition is configured to have a pH between about 7 and a pH of about 10.
71. The kit of any one of clauses 51 to 67, wherein the composition is configured to have a pH between about 7 and a pH of about 9.
72. The kit of any one of clauses 51 to 67, wherein the composition is configured to have a pH between about 7 and a pH of about 8.1.
73. The kit of any one of clauses 51 to 67, wherein the composition is configured to have a pH between about 7 and a pH of about 8.
74. The kit of any one of clauses 51 to 73, wherein the composition is configured to have an oxygen tension of less than about 30% of air.
75. The kit of any one of clauses 51 to 73, wherein the composition is configured to have an oxygen tension of between about 10% of air and about 30% of air.
76. The kit of any one of clauses 51 to 73, wherein the composition is configured to have an oxygen tension of between about 15% of air and about 25% of air.
77. The kit of any one of clauses 51 to 73, wherein the composition is configured to have an oxygen tension of between about 10% of air and about 20% of air.
78. The kit of any one of clauses 51 to 73, wherein the composition is configured to have an oxygen tension of less than about 10% of air.
79. The kit of any one of clauses 51 to 73, wherein the composition is configured to have an oxygen tension of between about 5% of air and about 10% of air.
80. The kit of any one of clauses 51 to 73, wherein the composition is configured to have an oxygen tension of between about 1% of air and about 10% of air.
81. The kit of any one of clauses 51 to 73, wherein the composition is configured to have an oxygen tension of between about 1% of air and about 5% of air.
82. A host-free microbial culture, comprising:
a composition, wherein the composition further comprises:
a Candidatus Liberibacter inoculum;
a Bacillus species; and
one or more antibiotics.
83. The host-free microbial culture of clause 82, wherein the one or more antibiotics are not effective to inhibit growth of the Candidatus Liberibacter inoculum.
84. The host-free microbial culture of clause 82 or clause 83, wherein the Bacillus species is genetically modified.
85. The host-free microbial culture of any one of clauses 82 to 84, wherein the Bacillus species is Bacillus subtilis.
86. The host-free microbial culture of any one of clauses 82 to 84, wherein the Bacillus species is Bacillus cereus.
87. The host-free microbial culture of any one of clauses 82 to 86, wherein the one or more antibiotics comprises at least one antibiotic selected from the group consisting of a vancomycin antibiotic, a streptomycin antibiotic, and a polymyxin antibiotic.
88. The host-free microbial culture of any one of clauses 82 to 86, wherein the one or more antibiotics comprises a vancomycin antibiotic.
89. The host-free microbial culture of clause 88, wherein the vancomycin antibiotic comprises a dose of about 50 μg/ml to about 99 μg/ml.
90. The host-free microbial culture of clause 88, wherein the vancomycin antibiotic comprises a dose of about 10 μg/ml to about 500 μg/ml.
91. The host-free microbial culture of clause 88, wherein the vancomycin antibiotic comprises a dose of about 50 μg/ml to about 250 μg/ml.
92. The host-free microbial culture of clause 88, wherein the vancomycin antibiotic comprises a dose of about 50 μg/ml to about 200 μg/ml.
93. The host-free microbial culture of clause 88, wherein the vancomycin antibiotic comprises a dose of at least 100 μg/ml.
94. The host-free microbial culture of any one of clauses 82 to 93, wherein the vancomycin antibiotic increases growth abundance of the Candidatus Liberibacter inoculum of at least a 3 fold change.
95. The host-free microbial culture of any one of clauses 82 to 93, wherein the vancomycin antibiotic increases growth abundance of the Candidatus Liberibacter inoculum of at least a 5 fold change.
96. The host-free microbial culture of any one of clauses 82 to 93, wherein the vancomycin antibiotic increases growth abundance of the Candidatus Liberibacter inoculum of at least a 7 fold change.
97. The host-free microbial culture of any one of clauses 82 to 93, wherein the vancomycin antibiotic increases growth abundance of the Candidatus Liberibacter inoculum of at least a 7.08 fold change.
98. The host-free microbial culture of any one of clauses 82 to 93, wherein the vancomycin antibiotic increases growth abundance of the Candidatus Liberibacter inoculum of at least a 8 fold change.
99. The host-free microbial culture of any one of clauses 82 to 93, wherein the vancomycin antibiotic increases growth abundance of the Candidatus Liberibacter inoculum of at least a 10 fold change.
100. The host-free microbial culture of any one of clauses 82 to 99, wherein the vancomycin antibiotic reduces growth abundance of the Candidatus Liberibacter inoculum.
101. The host-free microbial culture of any one of clauses 82 to 86, wherein the one or more antibiotics comprises a streptomycin antibiotic.
102. The host-free microbial culture of clause 101, wherein the streptomycin antibiotic comprises a dose of about 0.2 μg/ml to about 0.49 μg/ml.
103. The host-free microbial culture of clause 101, wherein the streptomycin antibiotic comprises a dose of about 0.01 μg/ml to about 10 μg/ml.
104. The host-free microbial culture of clause 101, wherein the streptomycin antibiotic comprises a dose of about 0.1 μg/ml to about 5 μg/ml.
105. The host-free microbial culture of clause 101, wherein the streptomycin antibiotic comprises a dose of about 0.1 μg/ml to about 2.5 μg/ml.
106. The host-free microbial culture of clause 101, wherein the streptomycin antibiotic comprises a dose of about 0.1 μg/ml to about 1 μg/ml.
107. The host-free microbial culture of clause 101, wherein the streptomycin antibiotic comprises a dose of at least 0.5 μg/ml.
108. The host-free microbial culture of any one of clauses 101 to 107, wherein the streptomycin antibiotic increases growth abundance of the Candidatus Liberibacter inoculum of at least a 3 fold change.
109. The host-free microbial culture of any one of clauses 101 to 107, wherein the streptomycin antibiotic increases growth abundance of the Candidatus Liberibacter inoculum of at least a 4 fold change.
110. The host-free microbial culture of any one of clauses 101 to 107, wherein the streptomycin antibiotic increases growth abundance of the Candidatus Liberibacter inoculum of at least a 5 fold change.
111. The host-free microbial culture of any one of clauses 101 to 107, wherein the streptomycin antibiotic increases growth abundance of the Candidatus Liberibacter inoculum of at least a 5.41 fold change.
112. The host-free microbial culture of any one of clauses 101 to 107, wherein the streptomycin antibiotic increases growth abundance of the Candidatus Liberibacter inoculum of at least a 6 fold change.
113. The host-free microbial culture of any one of clauses 101 to 107, wherein the streptomycin antibiotic increases growth abundance of the Candidatus Liberibacter inoculum of at least a 8 fold change.
114. The host-free microbial culture of any one of clauses 101 to 107, wherein the streptomycin antibiotic increases growth abundance of the Candidatus Liberibacter inoculum of at least a 10 fold change.
115. The host-free microbial culture of any one of clauses 101 to 114, wherein the streptomycin antibiotic reduces a growth abundance of the Candidatus Liberibacter inoculum.
116. The host-free microbial culture of any one of clauses 82 to 86, wherein the one or more antibiotics comprises a polymyxin antibiotic.
117. The host-free microbial culture of clause 116, wherein the polymyxin antibiotic comprises a dose level of 0.5 μg/ml to 4 μg/ml.
118. The host-free microbial culture of clause 116, wherein the polymyxin antibiotic comprises a dose level of 0.1 μg/ml to 10 μg/ml.
119. The host-free microbial culture of clause 116, wherein the polymyxin antibiotic comprises a dose level of 0.1 μg/ml to 8 μg/ml.
120. The host-free microbial culture of clause 116, wherein the polymyxin antibiotic comprises a dose level of 1 μg/ml to 5 μg/ml.
121. The host-free microbial culture of clause 116, wherein the polymyxin antibiotic comprises a dose level of 2 μg/ml to 5 μg/ml.
122. The host-free microbial culture of clause 116, wherein the polymyxin antibiotic comprises a dose of about 4 μg/ml.
123. The host-free microbial culture of any one of clauses 116 to 122, wherein the polymyxin antibiotic increases growth abundance of the Candidatus Liberibacter inoculum of at least a 2 fold change.
124. The host-free microbial culture of any one of clauses 116 to 122, wherein the polymyxin antibiotic increases growth abundance of the Candidatus Liberibacter inoculum of at least a 3 fold change.
125. The host-free microbial culture of any one of clauses 116 to 122, wherein the polymyxin antibiotic increases growth abundance of the Candidatus Liberibacter inoculum of at least a 3.71 fold change.
126. The host-free microbial culture of any one of clauses 116 to 122, wherein the polymyxin antibiotic increases growth abundance of the Candidatus Liberibacter inoculum of at least a 4 fold change.
127. The host-free microbial culture of any one of clauses 116 to 122, wherein the polymyxin antibiotic increases growth abundance of the Candidatus Liberibacter inoculum of at least a 5 fold change.
128. The host-free microbial culture of any one of clauses 116 to 122, wherein the polymyxin antibiotic increases growth abundance of the Candidatus Liberibacter inoculum of at least a 8 fold change.
129. The host-free microbial culture of any one of clauses 116 to 122, wherein the polymyxin antibiotic increases growth abundance of the Candidatus Liberibacter inoculum of at least a 10 fold change.
130. The host-free microbial culture of any one of clauses 116 to 129, wherein the polymyxin antibiotic reduces growth abundance of the Candidatus Liberibacter inoculum.
131. The host-free microbial culture of any one of clauses 82 to 130, wherein growth of the Candidatus Liberibacter in the culture is inversely correlated with the abundance of the Bacillus species in the culture.
132. The host-free microbial culture of any one of clauses 82 to 131, wherein the culture further comprises a plurality of nutrients.
133. The host-free microbial culture of clause 132, wherein the plurality of nutrients comprises a first plurality of nutrients and a second plurality of nutrients.
134. The host-free microbial culture of clause 133, wherein the second plurality of nutrients comprises at least one of trace minerals, salts, and vitamins to enhance the composition.
135. The host-free microbial culture of any one of clauses 132 to 134, wherein the plurality of nutrients comprises least one of nutrient selected from the group consisting of alpha-ketoglutaric acid, ACE buffer, potassium hydroxide, phosphate buffer, deionized water, and any combination thereof.
136. The host-free microbial culture of any one of clauses 132 to 135, wherein the plurality of nutrients comprises one or more trace minerals.
137. The host-free microbial culture of clause 136, wherein the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickel chloride, sodium tungstate, sodium selenite, and any combination thereof.
138. The host-free microbial culture of any one of clauses 132 to 137, wherein the plurality of nutrients comprises one or more salts.
139. The host-free microbial culture of clause 138, wherein the salts comprise at least one salt selected from the group consisting of potassium chloride, ammonium chloride, sodium hydrogen phosphate, calcium chloride, magnesium sulfate. and any combination thereof.
140. The host-free microbial culture of any one of clauses 132 to 139, wherein the plurality of nutrients comprises one or more vitamins
141. The host-free microbial culture of clause 140, wherein the vitamins comprise at least one vitamin selected from the group consisting of biotin, folic acid, pyridoxine hydrochloride, riboflavin, thiamine hydrochloride, nicotinic acid, DL-calcium pantothenate, vitamin B₁₂, p-aminobenzoic acid, thiocidic acid, and any combination thereof.
142. The host-free microbial culture of any one of clauses 82 to 141, wherein the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof.
143. The host-free microbial culture of any one of clauses 82 to 141, wherein the Candidatus Liberibacter inoculum is Candidatus Liberibacter asiaticus.
144. The host-free microbial culture of any one of clauses 82 to 141, wherein the Candidatus Liberibacter inoculum is Candidatus Liberibacter americanus.
145. The host-free microbial culture of any one of clauses 82 to 141, wherein the Candidatus Liberibacter inoculum is Candidatus Liberibacter africanus.
146. The host-free microbial culture of any one of clauses 82 to 141, wherein the Candidatus Liberibacter inoculum is Candidatus Liberibacter solanacearum.
147. A method of growing a host-free microbial culture, said method comprising the steps of:
inoculating a composition with a Candidatus Liberibacter inoculum;
combining a Bacillus species and the composition; and
treating the Candidatus Liberibacter inoculum with one or more antibiotics, wherein the one or more antibiotics are not effective to inhibit growth of the Candidatus Liberibacter inoculum.
148. The method of clause 147, wherein the Bacillus species is genetically modified.
149. The method of clause 147 or clause 148, wherein the Bacillus species is Bacillus subtilis.
150. The method of clause 147 or clause 148, wherein the Bacillus species is Bacillus cereus.
151. The method of any one of clauses 147 to 150, wherein the one or more antibiotics comprises at least one antibiotic selected from the group consisting of a vancomycin antibiotic, a streptomycin antibiotic, and a polymyxin antibiotic.
152. The method of any one of clauses 147 to 150, wherein the one or more antibiotics comprises a vancomycin antibiotic.
153. The method of clause 152, wherein the vancomycin antibiotic comprises a dose of about 50 μg/ml to about 99 μg/ml.
154. The method of clause 152, wherein the vancomycin antibiotic comprises a dose of about 10 μg/ml to about 500 μg/ml.
155. The method of clause 152, wherein the vancomycin antibiotic comprises a dose of about 50 μg/ml to about 250 μg/ml.
156. The method of clause 152, wherein the vancomycin antibiotic comprises a dose of about 50 μg/ml to about 200 μg/ml.
157. The method of clause 152, wherein the vancomycin antibiotic comprises a dose of at least 100 μg/ml.
158. The method of any one of clauses 152 to 157, wherein the vancomycin antibiotic increases growth abundance of the Candidatus Liberibacter inoculum of at least a 3 fold change.
159. The method of any one of clauses 152 to 157, wherein the vancomycin antibiotic increases growth abundance of the Candidatus Liberibacter inoculum of at least a 5 fold change.
160. The method of any one of clauses 152 to 157, wherein the vancomycin antibiotic increases growth abundance of the Candidatus Liberibacter inoculum of at least a 7 fold change.
161. The method of any one of clauses 152 to 157, wherein the vancomycin antibiotic increases growth abundance of the Candidatus Liberibacter inoculum of at least a 7.08 fold change.
162. The method of any one of clauses 152 to 157, wherein the vancomycin antibiotic increases growth abundance of the Candidatus Liberibacter inoculum of at least a 8 fold change.
163. The method of any one of clauses 152 to 157, wherein the vancomycin antibiotic increases growth abundance of the Candidatus Liberibacter inoculum of at least a 10 fold change.
164. The method of any one of clauses 152 to 164, wherein the vancomycin antibiotic reduces growth abundance of the Candidatus Liberibacter inoculum.
165. The method of any one of clauses 147 to 150, wherein the one or more antibiotics comprises a streptomycin antibiotic.
166. The method of clause 165, wherein the streptomycin antibiotic comprises a dose of about 0.2 μg/ml to about 0.49 μg/ml.
167. The method of clause 165, wherein the streptomycin antibiotic comprises a dose of about 0.01 μg/ml to about 10 μg/ml.
168. The method of clause 165, wherein the streptomycin antibiotic comprises a dose of about 0.1 μg/ml to about 5 μg/ml.
169. The method of clause 165, wherein the streptomycin antibiotic comprises a dose of about 0.1 μg/ml to about 2.5 μg/ml.
170. The method of clause 165, wherein the streptomycin antibiotic comprises a dose of about 0.1 μg/ml to about 1 μg/ml.
171. The method of clause 165, wherein the streptomycin antibiotic comprises a dose of at least 0.5 μg/ml.
172. The method of any one of clauses 165 to 171, wherein the streptomycin antibiotic increases growth abundance of the Candidatus Liberibacter inoculum of at least a 3 fold change.
173. The method of any one of clauses 165 to 171, wherein the streptomycin antibiotic increases growth abundance of the Candidatus Liberibacter inoculum of at least a 4 fold change.
174. The method of any one of clauses 165 to 171, wherein the streptomycin antibiotic increases growth abundance of the Candidatus Liberibacter inoculum of at least a 5 fold change.
175. The method of any one of clauses 165 to 171, wherein the streptomycin antibiotic increases growth abundance of the Candidatus Liberibacter inoculum of at least a 5.41 fold change.
176. The method of any one of clauses 165 to 171, wherein the streptomycin antibiotic increases growth abundance of the Candidatus Liberibacter inoculum of at least a 6 fold change.
177. The method of any one of clauses 165 to 171, wherein the streptomycin antibiotic increases growth abundance of the Candidatus Liberibacter inoculum of at least a 8 fold change.
178. The method of any one of clauses 165 to 171, wherein the streptomycin antibiotic increases growth abundance of the Candidatus Liberibacter inoculum of at least a 10 fold change.
179. The method of any one of clauses 165 to 178, wherein the streptomycin antibiotic reduces a growth abundance of the Candidatus Liberibacter inoculum.
180. The method of any one of clauses 147 to 150, wherein the one or more antibiotics comprises a polymyxin antibiotic.
181. The method of clause 180, wherein the polymyxin antibiotic comprises a dose level of 0.5 μg/ml to 4 μg/ml.
182. The method of clause 180, wherein the polymyxin antibiotic comprises a dose level of 0.1 μg/ml to 10 μg/ml.
183. The method of clause 180, wherein the polymyxin antibiotic comprises a dose level of 0.1 μg/ml to 8 μg/ml.
184. The method of clause 180, wherein the polymyxin antibiotic comprises a dose level of 1 μg/ml to 5 μg/ml.
185. The method of clause 180, wherein the polymyxin antibiotic comprises a dose level of 2 μg/ml to 5 μg/ml.
186. The method of clause 180, wherein the polymyxin antibiotic comprises a dose of about 4 μg/ml.
187. The method of any one of clauses 180 to 186, wherein the polymyxin antibiotic increases growth abundance of the Candidatus Liberibacter inoculum of at least a 2 fold change.
188. The method of any one of clauses 180 to 186, wherein the polymyxin antibiotic increases growth abundance of the Candidatus Liberibacter inoculum of at least a 3 fold change.
189. The method of any one of clauses 180 to 186, wherein the polymyxin antibiotic increases growth abundance of the Candidatus Liberibacter inoculum of at least a 3.71 fold change.
190. The method of any one of clauses 180 to 186, wherein the polymyxin antibiotic increases growth abundance of the Candidatus Liberibacter inoculum of at least a 4 fold change.
191. The method of any one of clauses 180 to 186, wherein the polymyxin antibiotic increases growth abundance of the Candidatus Liberibacter inoculum of at least a 5 fold change.
192. The method of any one of clauses 180 to 186, wherein the polymyxin antibiotic increases growth abundance of the Candidatus Liberibacter inoculum of at least a 8 fold change.
193. The method of any one of clauses 180 to 186, wherein the polymyxin antibiotic increases growth abundance of the Candidatus Liberibacter inoculum of at least a 10 fold change.
194. The method of any one of clauses 180 to 193, wherein the polymyxin antibiotic reduces growth abundance of the Candidatus Liberibacter inoculum.
195. The method of any one of clauses 147 to 194, wherein growth of the Candidatus Liberibacter in the culture is inversely correlated with the abundance of the Bacillus species in the culture.
196. The method of any one of clauses 147 to 195, wherein the method further comprises the step of combining a plurality of nutrients with the composition.
197. The method of clause 196, wherein the plurality of nutrients comprises a first plurality of nutrients and a second plurality of nutrients.
198. The method of clause 197, wherein the second plurality of nutrients comprises at least one of trace minerals, salts, and vitamins to enhance the composition.
199. The method of any one of clauses 196 to 198, wherein the plurality of nutrients comprises least one of nutrient selected from the group consisting of alpha-ketoglutaric acid, ACE buffer, potassium hydroxide, phosphate buffer, deionized water, and any combination thereof.
200. The method of any one of clauses 196 to 199, wherein the plurality of nutrients comprises one or more trace minerals.
201. The method of clause 200, wherein the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickel chloride, sodium tungstate, sodium selenite, and any combination thereof.
202. The method of any one of clauses 196 to 201, wherein the plurality of nutrients comprises one or more salts.
203. The method of clause 202, wherein the salts comprise at least one salt selected from the group consisting of potassium chloride, ammonium chloride, sodium hydrogen phosphate, calcium chloride, magnesium sulfate. and any combination thereof.
204. The method of any one of clauses 196 to 203, wherein the plurality of nutrients comprises one or more vitamins
205. The method of clause 204, wherein the vitamins comprise at least one vitamin selected from the group consisting of biotin, folic acid, pyridoxine hydrochloride, riboflavin, thiamine hydrochloride, nicotinic acid, DL-calcium pantothenate, vitamin B₁₂, p-aminobenzoic acid, thiocidic acid, and any combination thereof.
206. The method of any one of clauses 147 to 205, wherein the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof.
207. The method of any one of clauses 147 to 205, wherein the Candidatus Liberibacter inoculum is Candidatus Liberibacter asiaticus.
208. The method of any one of clauses 147 to 205, wherein the Candidatus Liberibacter inoculum is Candidatus Liberibacter americanus.
209. The method of any one of clauses 147 to 205, wherein the Candidatus Liberibacter inoculum is Candidatus Liberibacter africanus.
210. The method of any one of clauses 147 to 205, wherein the Candidatus Liberibacter inoculum is Candidatus Liberibacter solanacearum.
211. A method of growing a host-free microbial culture, said method comprising the steps of:
inoculating a composition with a Candidatus Liberibacter inoculum;
combining a Bacillus species and the composition; and
treating the Candidatus Liberibacter inoculum with one or more antibiotics, wherein the one or more antibiotics are effective to inhibit growth of the Candidatus Liberibacter inoculum.
212. The method of clause 211, wherein the Bacillus species is genetically modified.
213. The method of clause 211 or clause 212, wherein the Bacillus species is Bacillus subtilis.
214. The method of clause 211 or clause 212, wherein the Bacillus species is Bacillus cereus.
215. The method of any one of clauses 211 to 214, wherein the one or more antibiotics comprises at least one antibiotic selected from the group consisting of a vancomycin antibiotic, a streptomycin antibiotic, and a polymyxin antibiotic.
216. The method of any one of clauses 211 to 215, wherein the one or more antibiotics comprises a vancomycin antibiotic.
217. The method of clause 216, wherein the vancomycin antibiotic comprises a dose of about 50 μg/ml to about 99 μg/ml.
218. The method of clause 216, wherein the vancomycin antibiotic comprises a dose of about 10 μg/ml to about 500 μg/ml.
219. The method of clause 216, wherein the vancomycin antibiotic comprises a dose of about 50 μg/ml to about 250 μg/ml.
220. The method of clause 216, wherein the vancomycin antibiotic comprises a dose of about 50 μg/ml to about 200 μg/ml.
221. The method of clause 216, wherein the vancomycin antibiotic comprises a dose of at least 100 μg/ml.
222. The method of any one of clauses 216 to 221, wherein the vancomycin antibiotic increases growth abundance of the Candidatus Liberibacter inoculum of at least a 3 fold change.
223. The method of any one of clauses 216 to 221, wherein the vancomycin antibiotic increases growth abundance of the Candidatus Liberibacter inoculum of at least a 5 fold change.
224. The method of any one of clauses 216 to 221, wherein the vancomycin antibiotic increases growth abundance of the Candidatus Liberibacter inoculum of at least a 7 fold change.
225. The method of any one of clauses 216 to 221, wherein the vancomycin antibiotic increases growth abundance of the Candidatus Liberibacter inoculum of at least a 7.08 fold change.
226. The method of any one of clauses 216 to 221, wherein the vancomycin antibiotic increases growth abundance of the Candidatus Liberibacter inoculum of at least a 8 fold change.
227. The method of any one of clauses 216 to 221, wherein the vancomycin antibiotic increases growth abundance of the Candidatus Liberibacter inoculum of at least a 10 fold change.
228. The method of any one of clauses 216 to 227, wherein the vancomycin antibiotic reduces growth abundance of the Candidatus Liberibacter inoculum.
229. The method of any one of clauses 211 to 215, wherein the one or more antibiotics comprises a streptomycin antibiotic.
230. The method of clause 229, wherein the streptomycin antibiotic comprises a dose of about 0.2 μg/ml to about 0.49 μg/ml.
231. The method of clause 229, wherein the streptomycin antibiotic comprises a dose of about 0.01 μg/ml to about 10 μg/ml.
232. The method of clause 229, wherein the streptomycin antibiotic comprises a dose of about 0.1 μg/ml to about 5 μg/ml.
233. The method of clause 229, wherein the streptomycin antibiotic comprises a dose of about 0.1 μg/ml to about 2.5 μg/ml.
234. The method of clause 229, wherein the streptomycin antibiotic comprises a dose of about 0.1 μg/ml to about 1 μg/ml.
235. The method of clause 229, wherein the streptomycin antibiotic comprises a dose of at least 0.5 μg/ml.
236. The method of any one of clauses 229 to 235, wherein the streptomycin antibiotic increases growth abundance of the Candidatus Liberibacter inoculum of at least a 3 fold change.
237. The method of any one of clauses 229 to 235, wherein the streptomycin antibiotic increases growth abundance of the Candidatus Liberibacter inoculum of at least a 4 fold change.
238. The method of any one of clauses 229 to 235, wherein the streptomycin antibiotic increases growth abundance of the Candidatus Liberibacter inoculum of at least a 5 fold change.
239. The method of any one of clauses 229 to 235, wherein the streptomycin antibiotic increases growth abundance of the Candidatus Liberibacter inoculum of at least a 5.41 fold change.
240. The method of any one of clauses 229 to 235, wherein the streptomycin antibiotic increases growth abundance of the Candidatus Liberibacter inoculum of at least a 6 fold change.
241. The method of any one of clauses 229 to 235, wherein the streptomycin antibiotic increases growth abundance of the Candidatus Liberibacter inoculum of at least a 8 fold change.
242. The method of any one of clauses 229 to 235, wherein the streptomycin antibiotic increases growth abundance of the Candidatus Liberibacter inoculum of at least a 10 fold change.
243. The method of any one of clauses 229 to 242, wherein the streptomycin antibiotic reduces a growth abundance of the Candidatus Liberibacter inoculum.
244. The method of any one of clauses 211 to 215, wherein the one or more antibiotics comprises a polymyxin antibiotic.
245. The method of clause 244, wherein the polymyxin antibiotic comprises a dose level of 0.5 μg/ml to 4 μg/ml.
246. The method of clause 244, wherein the polymyxin antibiotic comprises a dose level of 0.1 μg/ml to 10 μg/ml.
247. The method of clause 244, wherein the polymyxin antibiotic comprises a dose level of 0.1 μg/ml to 8 μg/ml.
248. The method of clause 244, wherein the polymyxin antibiotic comprises a dose level of 1 μg/ml to 5 μg/ml.
249. The method of clause 244, wherein the polymyxin antibiotic comprises a dose level of 2 μg/ml to 5 μg/ml.
250. The method of clause 244, wherein the polymyxin antibiotic comprises a dose of about 4 μg/ml.
251. The method of any one of clauses 244 to 250, wherein the polymyxin antibiotic increases growth abundance of the Candidatus Liberibacter inoculum of at least a 2 fold change.
252. The method of any one of clauses 244 to 250, wherein the polymyxin antibiotic increases growth abundance of the Candidatus Liberibacter inoculum of at least a 3 fold change.
253. The method of any one of clauses 244 to 250, wherein the polymyxin antibiotic increases growth abundance of the Candidatus Liberibacter inoculum of at least a 3.71 fold change.
254. The method of any one of clauses 244 to 250, wherein the polymyxin antibiotic increases growth abundance of the Candidatus Liberibacter inoculum of at least a 4 fold change.
255. The method of any one of clauses 244 to 250, wherein the polymyxin antibiotic increases growth abundance of the Candidatus Liberibacter inoculum of at least a 5 fold change.
256. The method of any one of clauses 244 to 250, wherein the polymyxin antibiotic increases growth abundance of the Candidatus Liberibacter inoculum of at least a 8 fold change.
257. The method of any one of clauses 244 to 250, wherein the polymyxin antibiotic increases growth abundance of the Candidatus Liberibacter inoculum of at least a 10 fold change.
258. The method of any one of clauses 244 to 257, wherein the polymyxin antibiotic reduces growth abundance of the Candidatus Liberibacter inoculum. 259. The method of any one of clauses 211 to 258, wherein growth of the Candidatus Liberibacter in the culture is inversely correlated with the abundance of the Bacillus species in the culture.
260. The method of any one of clauses 211 to 259, wherein the method further comprises the step of combining a plurality of nutrients with the composition.
261. The method of clause 260, wherein the plurality of nutrients comprises a first plurality of nutrients and a second plurality of nutrients.
262. The method of clause 261, wherein the second plurality of nutrients comprises at least one of trace minerals, salts, and vitamins to enhance the composition.
263. The method of any one of clauses 260 to 262, wherein the plurality of nutrients comprises least one of nutrient selected from the group consisting of alpha-ketoglutaric acid, ACE buffer, potassium hydroxide, phosphate buffer, deionized water, and any combination thereof.
264. The method of any one of clauses 260 to 263, wherein the plurality of nutrients comprises one or more trace minerals.
265. The method of clause 264, wherein the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickel chloride, sodium tungstate, sodium selenite, and any combination thereof.
266. The method of any one of clauses 260 to 265, wherein the plurality of nutrients comprises one or more salts.
267. The method of clause 266, wherein the salts comprise at least one salt selected from the group consisting of potassium chloride, ammonium chloride, sodium hydrogen phosphate, calcium chloride, magnesium sulfate. and any combination thereof.
268. The method of any one of clauses 260 to 267, wherein the plurality of nutrients comprises one or more vitamins
269. The method of clause 268, wherein the vitamins comprise at least one vitamin selected from the group consisting of biotin, folic acid, pyridoxine hydrochloride, riboflavin, thiamine hydrochloride, nicotinic acid, DL-calcium pantothenate, vitamin Biz, p-aminobenzoic acid, thiocidic acid, and any combination thereof.
270. The method of any one of clauses 211 to 269, wherein the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof.
271. The method of any one of clauses 211 to 269, wherein the Candidatus Liberibacter inoculum is Candidatus Liberibacter asiaticus.
272. The method of any one of clauses 211 to 269, wherein the Candidatus Liberibacter inoculum is Candidatus Liberibacter americanus.
273. The method of any one of clauses 211 to 269, wherein the Candidatus Liberibacter inoculum is Candidatus Liberibacter africanus.
274. The method of any one of clauses 211 to 269, wherein the Candidatus Liberibacter inoculum is Candidatus Liberibacter solanacearum.
275. An assay for inhibiting growth of a Candidatus Liberibacter inoculum in a host-free microbial culture, said assay comprising the steps of
i) inoculating a composition with a Candidatus Liberibacter inoculum;
ii) combining the composition and a Bacillus species; and
iii) treating the Candidatus Liberibacter inoculum with one or more antibiotics, whereby the one or more antibiotics inhibit growth of the Candidatus Liberibacter inoculum in the host-free microbial culture.
276. An assay for growing a Candidatus Liberibacter inoculum in a host-free microbial culture, said assay comprising the steps of
i) inoculating a composition with a Candidatus Liberibacter inoculum;
ii) combining the composition and a Bacillus species with; and
iii) treating the Candidatus Liberibacter inoculum with one or more antibiotics, wherein the one or more antibiotics do not inhibit growth of the Candidatus Liberibacter inoculum in the host-free microbial culture.
277. A kit for inhibiting growth of a Candidatus Liberibacter inoculum, said kit comprising a host-free microbial culture, a Candidatus Liberibacter inoculum, a Bacillus species, one or more antibiotics, and an instruction for combination.
278. The assay or kit of any one of clauses 275 to 277, wherein the Bacillus species is genetically modified.
279. The assay or kit of any one of clauses 275 to 278, wherein the Bacillus species is Bacillus subtilis.
280. The assay or kit of any one of clauses 275 to 278, wherein the Bacillus species is Bacillus cereus.
281. The assay or kit of any one of clauses 275 to 280, wherein the one or more antibiotics comprises at least one antibiotic selected from the group consisting of a vancomycin antibiotic, a streptomycin antibiotic, and a polymyxin antibiotic.
282. The assay or kit of any one of clauses 275 to 280, wherein the one or more antibiotics comprises a vancomycin antibiotic.
283. The assay or kit of clause 282, wherein the vancomycin antibiotic comprises a dose of about 50 μg/ml to about 99 μg/ml.
284. The assay or kit of clause 282, wherein the vancomycin antibiotic comprises a dose of about 10 μg/ml to about 500 μg/ml.
285. The assay or kit of clause 282, wherein the vancomycin antibiotic comprises a dose of about 50 μg/ml to about 250 μg/ml.
286. The assay or kit of clause 282, wherein the vancomycin antibiotic comprises a dose of about 50 μg/ml to about 200 μg/ml.
287. The assay or kit of clause 282, wherein the vancomycin antibiotic comprises a dose of at least 100 μg/ml.
288. The assay or kit of any one of clauses 282 to 287, wherein the vancomycin antibiotic increases growth abundance of the Candidatus Liberibacter inoculum of at least a 3 fold change.
289. The assay or kit of any one of clauses 282 to 287, wherein the vancomycin antibiotic increases growth abundance of the Candidatus Liberibacter inoculum of at least a 5 fold change.
290. The assay or kit of any one of clauses 282 to 287, wherein the vancomycin antibiotic increases growth abundance of the Candidatus Liberibacter inoculum of at least a 7 fold change.
291. The assay or kit of any one of clauses 282 to 287, wherein the vancomycin antibiotic increases growth abundance of the Candidatus Liberibacter inoculum of at least a 7.08 fold change.
292. The assay or kit of any one of clauses 282 to 287, wherein the vancomycin antibiotic increases growth abundance of the Candidatus Liberibacter inoculum of at least a 8 fold change.
293. The assay or kit of any one of clauses 282 to 287, wherein the vancomycin antibiotic increases growth abundance of the Candidatus Liberibacter inoculum of at least a 10 fold change.
294. The assay or kit of any one of clauses 282 to 293, wherein the vancomycin antibiotic reduces growth abundance of the Candidatus Liberibacter inoculum.
295. The assay or kit of any one of clauses 275 to 280, wherein the one or more antibiotics comprises a streptomycin antibiotic.
296. The assay or kit of clause 295, wherein the streptomycin antibiotic comprises a dose of about 0.2 μg/ml to about 0.49 μg/ml.
297. The assay or kit of clause 295, wherein the streptomycin antibiotic comprises a dose of about 0.01 μg/ml to about 10 μg/ml.
298. The assay or kit of clause 295, wherein the streptomycin antibiotic comprises a dose of about 0.1 μg/ml to about 5 μg/ml.
299. The assay or kit of clause 295, wherein the streptomycin antibiotic comprises a dose of about 0.1 μg/ml to about 2.5 μg/ml.
300. The assay or kit of clause 295, wherein the streptomycin antibiotic comprises a dose of about 0.1 μg/ml to about 1 μg/ml.
301. The assay or kit of clause 295, wherein the streptomycin antibiotic comprises a dose of at least 0.5 μg/ml.
302. The assay or kit of any one of clauses 295 to 301, wherein the streptomycin antibiotic increases growth abundance of the Candidatus Liberibacter inoculum of at least a 3 fold change.
303. The assay or kit of any one of clauses 295 to 301, wherein the streptomycin antibiotic increases growth abundance of the Candidatus Liberibacter inoculum of at least a 4 fold change.
304. The assay or kit of any one of clauses 295 to 301, wherein the streptomycin antibiotic increases growth abundance of the Candidatus Liberibacter inoculum of at least a 5 fold change.
305. The assay or kit of any one of clauses 295 to 301, wherein the streptomycin antibiotic increases growth abundance of the Candidatus Liberibacter inoculum of at least a 5.41 fold change.
306. The assay or kit of any one of clauses 295 to 301, wherein the streptomycin antibiotic increases growth abundance of the Candidatus Liberibacter inoculum of at least a 6 fold change.
307. The assay or kit of any one of clauses 295 to 301, wherein the streptomycin antibiotic increases growth abundance of the Candidatus Liberibacter inoculum of at least a 8 fold change.
308. The assay or kit of any one of clauses 295 to 301, wherein the streptomycin antibiotic increases growth abundance of the Candidatus Liberibacter inoculum of at least a 10 fold change.
309. The assay or kit of any one of clauses 295 to 308, wherein the streptomycin antibiotic reduces a growth abundance of the Candidatus Liberibacter inoculum.
310. The assay or kit of any one of clauses 275 to 280, wherein the one or more antibiotics comprises a polymyxin antibiotic.
311. The assay or kit of clause 310, wherein the polymyxin antibiotic comprises a dose level of 0.5 μg/ml to 4 μg/ml.
312. The assay or kit of clause 310, wherein the polymyxin antibiotic comprises a dose level of 0.1 μg/ml to 10 μg/ml.
313. The assay or kit of clause 310, wherein the polymyxin antibiotic comprises a dose level of 0.1 μg/ml to 8 μg/ml.
314. The assay or kit of clause 310, wherein the polymyxin antibiotic comprises a dose level of 1 μg/ml to 5 μg/ml.
315. The assay or kit of clause 310, wherein the polymyxin antibiotic comprises a dose level of 2 μg/ml to 5 μg/ml.
316. The assay or kit of clause 310, wherein the polymyxin antibiotic comprises a dose of about 4 μg/ml.
317. The assay or kit of any one of clauses 310 to 316, wherein the polymyxin antibiotic increases growth abundance of the Candidatus Liberibacter inoculum of at least a 2 fold change.
318. The assay or kit of any one of clauses 310 to 316, wherein the polymyxin antibiotic increases growth abundance of the Candidatus Liberibacter inoculum of at least a 3 fold change.
319. The assay or kit of any one of clauses 310 to 316, wherein the polymyxin antibiotic increases growth abundance of the Candidatus Liberibacter inoculum of at least a 3.71 fold change.
320. The assay or kit of any one of clauses 310 to 316, wherein the polymyxin antibiotic increases growth abundance of the Candidatus Liberibacter inoculum of at least a 4 fold change.
321. The assay or kit of any one of clauses 310 to 316, wherein the polymyxin antibiotic increases growth abundance of the Candidatus Liberibacter inoculum of at least a 5 fold change.
322. The assay or kit of any one of clauses 310 to 316, wherein the polymyxin antibiotic increases growth abundance of the Candidatus Liberibacter inoculum of at least a 8 fold change.
323. The assay or kit of any one of clauses 310 to 316, wherein the polymyxin antibiotic increases growth abundance of the Candidatus Liberibacter inoculum of at least a 10 fold change.
324. The assay or kit of any one of clauses 310 to 323, wherein the polymyxin antibiotic reduces growth abundance of the Candidatus Liberibacter inoculum.
325. The assay or kit of any one of clauses 275 to 324, wherein growth of the Candidatus Liberibacter in the culture is inversely correlated with the abundance of the Bacillus species in the culture.
326. The assay or kit of any one of clauses 275 to 325, wherein the assay further comprises combining a plurality of nutrients with the composition.
327. The assay or kit of clause 326, wherein the plurality of nutrients comprises a first plurality of nutrients and a second plurality of nutrients.
328. The assay or kit of clause 327, wherein the second plurality of nutrients comprises at least one of trace minerals, salts, and vitamins to enhance the composition.
329. The assay or kit of any one of clauses 326 to 328, wherein the plurality of nutrients comprises least one of nutrient selected from the group consisting of alpha-ketoglutaric acid, ACE buffer, potassium hydroxide, phosphate buffer, deionized water, and any combination thereof.
330. The assay or kit of any one of clauses 326 to 329, wherein the plurality of nutrients comprises one or more trace minerals.
331. The assay or kit of clause 330, wherein the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickel chloride, sodium tungstate, sodium selenite, and any combination thereof.
332. The assay or kit of any one of clauses 326 to 331, wherein the plurality of nutrients comprises one or more salts.
333. The assay or kit of clause 332, wherein the salts comprise at least one salt selected from the group consisting of potassium chloride, ammonium chloride, sodium hydrogen phosphate, calcium chloride, magnesium sulfate. and any combination thereof.
334. The assay or kit of any one of clauses 324 to 333, wherein the plurality of nutrients comprises one or more vitamins
335. The assay or kit of clause 334, wherein the vitamins comprise at least one vitamin selected from the group consisting of biotin, folic acid, pyridoxine hydrochloride, riboflavin, thiamine hydrochloride, nicotinic acid, DL-calcium pantothenate, vitamin B₁₂, p-aminobenzoic acid, thiocidic acid, and any combination thereof.
336. The assay or kit of any one of clauses 275 to 335, wherein the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof.
337. The assay or kit of any one of clauses 275 to 335, wherein the Candidatus Liberibacter inoculum is Candidatus Liberibacter asiaticus.
338. The assay or kit of any one of clauses 275 to 335, wherein the Candidatus Liberibacter inoculum is Candidatus Liberibacter americanus.
339. The assay or kit of any one of clauses 275 to 335, wherein the Candidatus Liberibacter inoculum is Candidatus Liberibacter africanus.
340. The assay or kit of any one of clauses 275 to 335, wherein the Candidatus Liberibacter inoculum is Candidatus Liberibacter solanacearum.
341. An assay for detecting a HLB infection in a plant, said assay comprising the steps of
i) obtaining a tissue from the plant;
ii) incubating the tissue in a culture; and
iii) quantifying DNA of a bacteria in the cultured tissue, whereby a quantity of the bacterial DNA in the cultured tissue indicates the HLB infection in the plant.
342. The assay of clause 341, wherein the culture comprises basal PBS.
343. The assay of clause 341 or clause 342, wherein the culture comprises one or more of Na₂HPO₄, KH₂PO₄, KCl, NaCl, CaCl₂, MgCl₂, and any combination thereof.
344. The assay of any one of clauses 341 to 343, wherein the tissue is a leaf.
345. The assay of clause 344, wherein the leaf is a leaf disc.
346. The assay of clause 345, wherein the leaf disc is cut from a disc midrib.
347. The assay of any one of clauses 341 to 346, wherein the quantification of DNA is performed via a PCR assay.
348. The assay of clause 347, wherein the PCR assay utilizes a primer.
349. The assay of clause 348, wherein the primer comprises SEQ ID NO: 31.
350. The assay of clause 348, wherein the primer comprises SEQ ID NO: 32.
351. The assay of clause 348, wherein the primer comprises SEQ ID NO: 33.
352. The assay of clause 348, wherein the primer comprises SEQ ID NO: 34.
353. The assay of clause 348, wherein the primer comprises SEQ ID NO: 35.
354. The assay of clause 348, wherein the primer comprises SEQ ID NO: 36.
355. The assay of clause 348, wherein the primer comprises SEQ ID NO: 37.
356. The assay of clause 348, wherein the primer comprises SEQ ID NO: 38.
357. The assay of clause 348, wherein the primer comprises SEQ ID NO: 39.
358. The assay of clause 348, wherein the primer comprises SEQ ID NO: 40.
359. The assay of clause 348, wherein the primer comprises SEQ ID NO: 41.
360. The assay of clause 348, wherein the primer comprises SEQ ID NO: 42.
361. The assay of clause 348, wherein the primer consists essentially of SEQ ID NO: 31.
362. The assay of clause 348, wherein the primer consists essentially of SEQ ID NO: 32.
363. The assay of clause 348, wherein the primer consists essentially of SEQ ID NO: 33.
364. The assay of clause 348, wherein the primer consists essentially of SEQ ID NO: 34.
365. The assay of clause 348, wherein the primer consists essentially of SEQ ID NO: 35.
366. The assay of clause 348, wherein the primer consists essentially of SEQ ID NO: 36.
367. The assay of clause 348, wherein the primer consists essentially of SEQ ID NO: 37.
368. The assay of clause 348, wherein the primer consists essentially of SEQ ID NO: 38.
369. The assay of clause 348, wherein the primer consists essentially of SEQ ID NO: 39.
370. The assay of clause 348, wherein the primer consists essentially of SEQ ID NO: 40.
371. The assay of clause 348, wherein the primer consists essentially of SEQ ID NO: 41.
372. The assay of clause 348, wherein the primer consists essentially of SEQ ID NO: 42.
373. The assay of clause 348, wherein the primer consists of SEQ ID NO: 31.
374. The assay of clause 348, wherein the primer consists of SEQ ID NO: 32.
375. The assay of clause 348, wherein the primer consists of SEQ ID NO: 33.
376. The assay of clause 348, wherein the primer consists of SEQ ID NO: 34.
377. The assay of clause 348, wherein the primer consists of SEQ ID NO: 35.
378. The assay of clause 348, wherein the primer consists of SEQ ID NO: 36.
379. The assay of clause 348, wherein the primer consists of SEQ ID NO: 37.
380. The assay of clause 348, wherein the primer consists of SEQ ID NO: 38.
381. The assay of clause 348, wherein the primer consists of SEQ ID NO: 39.
382. The assay of clause 348, wherein the primer consists of SEQ ID NO: 40.
383. The assay of clause 348, wherein the primer consists of SEQ ID NO: 41.
384. The assay of clause 348, wherein the primer consists of SEQ ID NO: 42.
385. The assay of any one of clauses 341 to 384, wherein the quantification of the DNA is performed after incubation of the tissue for 3 days.
386. A kit for detecting a HLB infection in a plant, said kit comprising a culture and an instruction for incubating a tissue in the culture.
387. The kit of clause 386, wherein the culture comprises basal PBS.
388. The kit of clause 386 or clause 387, wherein the culture comprises one or more of Na₂HPO₄, KH₂PO₄, KCl, NaCl, CaCl₂, MgCl₂, and any combination thereof.
389. The kit of any one of clauses 386 to 388, wherein the tissue is a leaf.
390. The kit of clause 389, wherein the leaf is a leaf disc.
391. The kit of clause 390, wherein the leaf disc is cut from a disc midrib.

### BRIEF DESCRIPTION OF THE DRAWINGS

FIG. 1 shows a schematic representation of a biofilm reactor (MBR) system that can be utilized to culture Candidatus Liberibacter pathogens, as disclosed herein.

FIG. 2A shows validation of the presence and growth of “Ca. L. asiaticus” in biofilm cultures. Biofilm were cultured in the MBRs without aeration and at pH 7.0, wherein FIG. 2A shows PCR amplification of 1160 bp fragment(s) of the “Ca. L. asiaticus” 16S rDNA gene using specific primers O11 and O12c.

FIG. 2B shows validation and quantification of “Ca. L. asiaticus” growth in the MBR at the 5th transfer using qPCR with a probe specific for the 16S rDNA of “Ca. L. asiaticus”.

FIG. 3A illustrates validation of the presence and growth of ‘C. L. asiaticus’ in biofilm cultures over many transfers and independent repetitions. FIG. 3A in combination with FIG. 3B particularly shows validation of ‘C. L. asiaticus’ presence and growth in the MBR at the 7^{th}, 8^{th} and 9^{th} transfers with the PCR using conventional specific primers for ‘C. L. asiaticus’ (O11 and O12c) and qPCR with a specific probe for 16S rRNA of ‘C. L. asiaticus’.

FIG. 3B also illustrate validation of the presence and growth of ‘C. L. asiaticus’ in biofilm cultures over many transfers and independent repetitions. FIG. 3B in combination with FIG. 3A particularly shows validation of ‘C. L. asiaticus’ presence and growth in the MBR at the 7^{th}, 8^{th} and 9^{th} transfers with the PCR using conventional specific primers for ‘C. L. asiaticus’ (O11 and O12c) and qPCR with a specific probe for 16S rRNA of ‘C. L. asiaticus’.

FIG. 3C illustrates validation of ‘C. L. asiaticus’ presence and growth in a newly repeated MBR experiment at a different laboratory. FIG. 3C in combination with FIG. 3D particularly shows the increase of ‘C. L. asiaticus’ genome equivalents in an MBR inoculated with a plant extract from ‘C. L. asiaticus’-infected Hamlin over 14 days of operation clearly demonstrates the growth of ‘C. L. asiaticus’ in a biofilm reactor. Tracks 2 and 4: initial inoculation; tracks 1 and 3: 14 days after inoculation. Tracks 1, 3, 5: initial inoculation; tracks 2. 4, 6: 14 days after inoculation; track M: 1-kb marker.

FIG. 3D illustrates validation of ‘C. L. asiaticus’ presence and growth in a newly repeated MBR experiment at a different laboratory. FIG. 3D in combination with FIG. 3C particularly shows the increase of ‘C. L. asiaticus’ genome equivalents in an MBR inoculated with a plant extract from ‘C. L. asiaticus’-infected Hamlin over 14 days of operation clearly demonstrates the growth of ‘C. L. asiaticus’ in a biofilm reactor. Tracks 2 and 4: initial inoculation; tracks 1 and 3: 14 days after inoculation. Tracks 1, 3, 5: initial inoculation; tracks 2. 4, 6: 14 days after inoculation; track M: 1-kb marker.

FIG. 4A shows validation and quantification of the presence of ‘C. L. asiaticus’ 16S rRNA gene among the total 16S rRNA genes amplified from the biofilm cultures. FIG. 4A particularly shows the total of about 1500 bp (denoted with directional arrow) 16S rRNA gene fragments from microbial communities were amplified using universal primer 27F/1492r.

FIG. 4B shows these 16S rRNA gene fragments were purified and used as the template for the PCR reactions using ‘C. L. asiaticus’-specific primers (O11/O12c). The specific band of 1160 bp (also denoted with directional arrow) was amplified from the total 16S rRNA gene fragments of biofilm cultures and ‘C. L. asiaticus’-infected citrus Hamlin but not from a mock community which did not contain ‘C. L. asiaticus’.

FIG. 4C shows validation and quantification of ‘C. L. asiaticus’ 16S rRNA gene in a total 16S rRNA genes isolated from the microbial community of biofilm cultures using qPCR with a specific probe for 16S rRNA of ‘C. L. asiaticus’.

FIG. 5A shows the evident growth of a particular Candidatus Liberibacter pathogen, i.e., “Ca. L. asiaticus” despite still being a minor member of the biofilm community.

FIG. 5B shows inactivated inoculum 56 of Ca. L. asiaticus, which led to accumulation of a white layer of protein precipitate.

FIG. 5C shows a scanning electron micrographs of “Ca. L. asiaticus”-containing biofilms to clearly illustrate the presence of multiple microorganisms embedded within extracellular polymeric substance.

FIG. 5D shows longer, rod-shaped bacteria with the same morphology as L. crescens so as to illustrate an ease of detection as a minor biofilm component.

FIG. 5E shows microbial community profiles of “Ca. L. asiaticus”-containing biofilm and planktonic culture revealed that circa 99% of the population of the cultures, both biofilm and planktonic, was a Chryseobacterium species.

FIG. 6A shows a plot of pH and oxygen tension on the growth of “Ca. L. asiaticus” in a mixed culture with other biofilm microorganisms. FIG. 6A indicates no significant growth was detected at pH 6, but there was growth at pH 7 and 8. All cultures were grown without aeration.

FIG. 6B in corroboration with FIG. 2A shows higher genome equivalent levels of “Ca. L. asiaticus” were measured in the cultures grown under 10% oxygen tension than at other oxygen tensions. No growth was detected in the culture lacking oxygen or in the culture with an oxygen-saturated condition. All cultures were grown at pH 7.

FIG. 7 shows seasonal development of citrus and bacterial loads during critical stages of the growing season. Schematic of citrus tree annual cycle in Florida, USA. Observed seasonal variability of “Ca. L. asiaticus” in the shoot matches the elevated photosynthetic activity during main flush development in the spring during blooming and minor flush development during the summer and early fall. In the late fall and winter, “Ca. L. asiaticus” is observed at comparatively lower titers in the shoot.

FIGS. 8A-8B show primary metabolite profiles in leaves from healthy or “Ca. L. asiaticus”-infected plants following incubation with glucose (March). Heatmaps show differences between healthy or “Ca. L. asiaticus”-infected citrus leaves harvested in March after three days of incubation in the presence or absence of glucose. Sterilized leaf discs were prepared and primary metabolites extracted and analyzed as described in materials and methods. (A) Autoscaled relative abundances of a set of reliably identified metabolites are colored blue (lower abundance) to red (higher abundance) in the analysis and are noted in the corresponding symbols in the figures. Heatmap rows correspond to the analytes (n=4-5), and (B) averages of autoscaled signal intensities from each treatment group. Hierarchical clustering with Euclidean distance as similarity measure and Ward's linkage as clustering algorithm was performed using MetaboAnalyst 4.0. Absolutely consistent sample groupings were not observed suggesting “Ca. L. asiaticus” does not strongly affect primary metabolite profiles in young, flowering trees in the spring.

FIGS. 9A-9B show primary metabolite profiles in leaves from healthy or “Ca. L. asiaticus”-infected plants following incubation with glucose (June). Heatmaps show differences between healthy or “Ca. L. asiaticus”-infected citrus leaves harvested in June after three days of incubation in the presence or absence of glucose. Sterilized leaf discs were prepared and primary metabolites extracted and analyzed as described in materials and methods. (A) Autoscaled relative abundances of a set of reliably identified metabolites were colored blue (lower abundance) to red (higher abundance) in the analysis and are noted in the corresponding symbols in the figures. Heatmap rows correspond to the analytes (n=4-5), and (B) averages of autoscaled signal intensities from each treatment group. Hierarchical clustering with Euclidean distance as similarity measure and Ward's linkage as clustering algorithm was performed using MetaboAnalyst 4.0. Absolutely consistent sample groupings were not observed suggesting “Ca. L. asiaticus” does not strongly affect primary metabolite profiles in the early summer. However, moderate signal reduction of numerous metabolites in infected tissue is consistent with an impact of infection on leaf metabolic status and capacity.

FIG. 10 shows schematic of experimental design for analyzing “Ca. L. asiaticus” DNA replication in leaf discs. Infected leaves were collected from several branches to reduce overall sample variability. Leaves were surface sterilized and rinsed using a 3-step protocol (1-70% ethanol; 2-10% bleach+0.01% Tween-20; 3—sterile water). After preparation, leaf discs were pooled and divided into groups of 5 discs to normalize the number of bacteria per sample, necessary to reduce sample variability originating from uneven distribution of “Ca. L. asiaticus” in infected tissue. After incubation, samples were processed and analyzed as described. White dots indicate uneven distribution of bacteria within the leaf.

FIGS. 11A-11C shows distribution of “Ca. L. asiaticus” in the shoot and within citrus leaves. (A) Distribution of “Ca. L. asiaticus” among leaves collected from different branches. Two leaf discs were collected along the midribs of twelve leaves from different branches and GE measured by qPCR. “Ca. L. asiaticus” is unevenly distributed; some leaves were not colonized with “Ca. L. asiaticus” at detectable levels. Data from a representative experiment is shown. (B) Distribution of “Ca. L. asiaticus” along the midrib of infected leaves. Five infected leaves (L1-L5) from different branches were tested for distribution of “Ca. L. asiaticus” along the midrib. Two leaf discs were prepared by punching discs along the midrib for each section of the leaf from the apex of the leaf to the base (insert). The results show uneven distribution of “Ca. L. asiaticus” along the midribs of individual leaves. Data from a representative experiment is shown. (C) Average “Ca. L. asiaticus” DNA content in leaf tissue harvested in the spring, summer, or fall. Mixing leaf discs collected from several leaves assures detectable “Ca. L. asiaticus” DNA regardless of time of harvest.

FIGS. 12A-12D show “Ca. L. asiaticus” DNA replication in citrus leaf discs is activated by glucose availability under microaerobic conditions. To determine the capacity of “Ca. L. asiaticus” to undergo DNA replication in situ, “Ca. L. asiaticus”-infected leaf discs were incubated for three days in plain PBS (control) or PBS supplemented with 10 mM glucose under different oxygen conditions and GE measured. (A) Incubation in 20% O₂ did not support “Ca. L. asiaticus” DNA replication within the leaf regardless of nutrient condition. (B) Incubation of infected leaf discs in 10% O₂ in the presence of glucose resulted in a significant increase in GE as compared to the d0 control (P=0.0102) and between samples incubated with or without glucose for 3 days (P=0.0345), suggesting lower O₂ tension stimulated “Ca. L. asiaticus” replication of DNA and that presence of glucose promotes DNA synthesis. (C) The ability of “Ca. L. asiaticus” to undergo DNA replication was measured using a dose-response assay. Viable or autoclaved (“inactivated”) “Ca. L. asiaticus”-infected leaf discs were incubated for three days in plain PBS (control), or PBS supplemented with 0.5 or 10 mM glucose, leading to dose-dependent increases in GE (d0 vs d3, 10 mM glucose: P=0.005; d3, 0.5 vs 10 mM: P=0.0411). Autoclaved leaves did not show any response to glucose. Asterisk denotes a significant difference (one-way ANOVA, Tukey HSD post-hoc test) between indicated samples. (D) Confirmation of absence of pgi in “Ca. L. asiaticus” by conventional PCR using DNA template extracted from healthy or “Ca. L. asiaticus”-infected leaves, and DNA extracted from L. crescens. These results are consistent with the absence of pgi in the “Ca. L. asiaticus” strain used in this study.

FIG. 13 shows the effect of amikacin on “Ca. L. asiaticus” DNA replication in leaf discs incubated with glucose. To assess the effect of amikacin on “Ca. L. asiaticus” DNA replication in situ, “Ca. L. asiaticus”-infected leaf discs were incubated for three days in plain PBS (control), or PBS supplemented with 10 mM glucose in the absence or presence of 100 μg/ml amikacin. Incubation of infected leaf disc in PBS supplemented with 10 mM glucose and 100 μg/ml amikacin under 10% 02 resulted in significant enhancement of “Ca. L. asiaticus” DNA replication compared to incubation with glucose alone. Data was collected from three independent experiments done with three technical replicates. Asterisk denotes a significant difference (one-way Anova test, Tukey HSD post-hoc test) among the indicated test groups.

FIG. 14A-14B show the effect of glucose treatment on abundance of selected saccharides. Relative abundances of selected saccharides in March (A) or June (B) in healthy or infected leaves incubated for 3 days with or without glucose. Relative abundance for each metabolite is scaled to the signal in healthy leaves incubated in PBS (average of h-PBS=100%). Depicted data illustrate the mean±SD (n=4-5). Observed abundance shifts varied between treatments and season with “Ca. L. asiaticus”-infection resulting in elevated levels of fructose 6-P, glucose 1-P, glucose 6-P, and sucrose in March but not in June. Asterisks indicate P<0.005 (ANOVA).

FIGS. 15A-15B show the effect of glucose treatment on abundance of selected TCA cycle intermediates. Relative abundance of selected TCA cycle intermediates in March (A) or June (B) in healthy or infected leaves incubated for 3 days with or without glucose. Relative abundance for each metabolite is scaled to the signal in healthy leaves incubated in PBS (average of h-PBS=100%). Depicted data illustrate the mean±SD (n=4-5). Observed abundance shifts showed extensive variability between treatments and season.

FIGS. 16A-16C show the selective measurement of “Ca. L. asiaticus” GE by qPCR. A qPCR protocol for absolute quantification of “Ca. L. asiaticus” DNA was designed based on amplification of the hypothetical gene CD16-00155 (“Ca. L. asiaticus”, strain A4). Primer pairs used for amplification were assessed by qPCR for selectivity and sensitivity by comparison to a conventional primer set used to detect “Ca. L. asiaticus” 16s rDNA. DNA extracted from a healthy citrus plant was used as a negative control. (A) Comparison of PCR amplification from 50 ng tDNA template extracted from healthy (control) or “Ca. L. asiaticus”-infected leaves, L. crescens or Coxiella burnetii was used to evaluate the specificity of this primer set. The broken line indicates background as measured by signal obtained in the no-template control (NTC). (Insert) DNA gel loaded with qPCR reaction product corresponding to the quantitative analysis, confirming absence of a detectable signal in samples other than those containing “Ca. L. asiaticus”. (B-C) qPCR amplification of serially diluted “Ca. L. asiaticus” DNA template was used to compare amplification kinetics between primers targeting CD16-00155 or 16S rDNA. The results confirm that qPCR-based analysis of CD16-00155 allows selective and highly sensitive detection of “Ca. L. asiaticus”.

FIG. 17 shows a map of plasmid standard used for absolute quantification of “Ca. L. asiaticus” based on gene CD16-00155. To quantify the absolute load of “Ca. L. asiaticus” the target sequence of the hypothetical gene CD16-00155 (“Ca. L. asiaticus”, strain A4), was cloned into the pCR™4-TOPO® TA vector (Invitrogen, Carlsbad, Calif.) and transformed into E. coli TOP10 cells. Plasmid copy numbers were calculated based on the molecular weight of the plasmid.

FIG. 18 shows assessment of the ability of “Ca. L. asiaticus” to replicate in citrus leaves under different oxygen conditions. To determine the capacity of “Ca. L. asiaticus” to undergo DNA replication in situ under different microaerobic conditions, “Ca. L. asiaticus”-infected leaf discs were incubated for three days in plain PBS (control) or PBS supplemented with 10 mM glucose under conditions of either 2.5% or 10% O₂. Incubation in 2.5% 02 did not support “Ca. L. asiaticus” DNA replication within the leaf regardless of nutrient condition. However, when the “Ca. L. asiaticus”-infected leaf discs were incubated in PBS supplemented with 10 mM glucose under 10% O₂, a statistically significant increase in “Ca. L. asiaticus” DNA replication was observed compared to the d0 control (P=0.0001). Depicted data illustrate the mean+/−SEM. Data was collected from two independent experiments done with three technical replicates. Asterisk denotes a significant difference (one-way ANOVA test, Tukey HSD post-hoc test) among the indicated test groups.

FIG. 19 shows maintenance of tissue integrity and natural leaf color over three days of incubation in the absence of light. The effect of incubating leaf discs without light was assessed by evaluation of tissue color. Non-incubated leaf discs, or leaf discs incubated in plain PBS or PBS supplemented with 10 mM glucose for three days in the absence of light maintain natural color. Leaf discs show some bruising due to handling.

FIG. 20 shows growth of “Ca. L. asiaticus” in host-free mixed microbial cultures inoculated with “Ca. L. asiaticus”-infected ground ACP. The “Ca. L. asiaticus” genome equivalents (GE) were compared at time zero and after 7 days. The GE are averages of three biological replicates, and the error bars are the standard deviations. In the first cycle the host-free mixed microbial cultures were inoculated with homogenized psyllids. In the following cycles, the host-free mixed culture from the previous cycle was used as the inoculum. The stars indicate a significant statistical difference between day zero and day seven (one-way ANOVA).

FIGS. 21A-21D show effects of (A) vancomycin, (B) streptomycin, (C) polymyxin B, and (D) kasugamyicn on “Ca. L. asiaticus” growth. The fold change is equal to the change in “Ca. L. asiaticus” GE (after 7 days) in an antibiotic-treated host-free mixed microbial culture divided by the change in “Ca. L. asiaticus” GE (after 7 days) in a non-antibiotic control. The fold changes are averages of three biological replicates, and the error bars are standard deviations. One-way ANOVA and Dunnett's test were used for statistical comparisons to the no-antibiotic control. The stars indicate a significant statistical difference in “Ca. L. asiaticus” GE between an antibiotic-treated host-free mixed culture and the no-antibiotic control. NS refers to no significant difference.

FIGS. 22A-22B show (A) The effect of antibiotic treatment on “Ca. L. asiaticus” growth and (B) the distribution of bacterial families for each antibiotic treatment. The sample marked with a black star is the untreated culture at time zero, and the sample marked with a black circle is the sample that was treated with 0.25 μg/1 of kasugamycin. Van: vancomycin; Str: streptomycin; Pmb: polymyxin B.

FIGS. 23A-23B show principal component analysis of bacterial communities affected by antibiotic treatments (Bray-Curtis dissimilarity metrics). The samples were treated with 25, 35, 50, 75, or 100 μg/ml of vancomycin (blue) (V25, V35, V50, V75, or V100, respectively); 0.02, 0.05, 0.5, 1, or 2 μg/ml of streptomycin (green) (50.02, 50.05, 50.5, 51 or S2, respectively); 0.5, 2, or 4 μg/ml of polymyxin B (red) (P0.5, P2, or P4, respectively); or 0.25 μg/ml of kasugamycin (kag) (black). The no-treatment sample (orange) (N) and time zero (light blue) (TO) are included. A) The correlation circle. B) The observation plot.

FIG. 24 shows an example flow chart of an embodiment described herein.

FIG. 25 shows species (OTU) accumulation curves of antibiotic-treated and untreated “Ca. L. asiaticus” host-free mixed cultures.

FIG. 26 shows the sequences for SEQ ID NOS. 1-30.

FIG. 27 shows the sequences for SEQ ID NOS. 45-58.

### DETAILED DESCRIPTION

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Various embodiments of the invention are described herein as follows. In one embodiment described herein, a bacterial growth medium is provided. The bacterial growth medium comprises a composition, wherein the composition further comprises: a plurality of nutrients and a Candidatus Liberibacter inoculum.

In another embodiment described herein, a method of growing a bacterium is provided. The method comprises the steps of: i) inoculating a composition using a Candidatus Liberibacter bacteria; and ii) physiochemically adjusting the composition to comprise a pH between 7 and 12 and an oxygen tension of less than about 30% of air of the composition.

In yet another embodiment described herein, a kit for a bacterial growth medium is provided. The kit comprises a composition comprising a plurality of nutrients, a Candidatus Liberibacter inoculum, and an instruction for combination of the plurality of nutrients and the Candidatus Liberibacter inoculum.

In another embodiment described herein, a host-free microbial culture is provided. The host-free microbial culture comprises a composition, wherein the composition further comprises: a Candidatus Liberibacter inoculum; a Bacillus species; and one or more antibiotics.

In yet another embodiment described herein, a method of growing a host-free microbial culture is provided. The method comprises the steps of: inoculating a composition with a Candidatus Liberibacter inoculum; combining a Bacillus species and the composition; and treating the Candidatus Liberibacter inoculum with one or more antibiotics, wherein the one or more antibiotics are not effective to inhibit growth of the Candidatus Liberibacter inoculum.

In another embodiment described herein, a method of growing a host-free microbial culture is provided. The method comprises the steps of: inoculating a composition with a Candidatus Liberibacter inoculum; combining a Bacillus species and the composition; and treating the Candidatus Liberibacter inoculum with one or more antibiotics, wherein the one or more antibiotics are effective to inhibit growth of the Candidatus Liberibacter inoculum.

In yet another embodiment described herein, an assay for inhibiting growth of a Candidatus Liberibacter inoculum in a host-free microbial culture is provided. The assay comprises the steps of i) inoculating a composition with a Candidatus Liberibacter inoculum; ii) combining the composition and a Bacillus species; and iii) treating the Candidatus Liberibacter inoculum with one or more antibiotics, whereby the one or more antibiotics inhibit growth of the Candidatus Liberibacter inoculum in the host-free microbial culture.

In another embodiment described herein, an assay for growing a Candidatus Liberibacter inoculum in a host-free microbial culture is provided. The assay comprises the steps of i) inoculating a composition with a Candidatus Liberibacter inoculum; ii) combining the composition and a Bacillus species with; and iii) treating the Candidatus Liberibacter inoculum with one or more antibiotics, wherein the one or more antibiotics do not inhibit growth of the Candidatus Liberibacter inoculum in the host-free microbial culture.

In yet another embodiment described herein, a kit for inhibiting growth of a Candidatus Liberibacter inoculum is provided. The kit comprises a host-free microbial culture, a Candidatus Liberibacter inoculum, a Bacillus species, one or more antibiotics, and an instruction for combination.

In another embodiment described herein, an assay for detecting a HLB infection in a plant is provided. The assay comprises the steps of: i) obtaining a tissue from the plant; ii) incubating the tissue in a culture; and iii) quantifying DNA of a bacteria in the cultured tissue, whereby a quantity of the bacterial DNA in the cultured tissue indicates the HLB infection in the plant.

In yet another embodiment described herein, a kit for detecting a HLB infection in a plant is provided. The kit comprises a culture and an instruction for incubating a tissue in the culture.

In one aspect, a bacterial growth medium is provided. The bacterial growth medium comprises a composition, wherein the composition further comprises: a plurality of nutrients and a Candidatus Liberibacter inoculum.

In some embodiments, the composition is configured as a solidified media with an adjusted neutral pH. In various embodiments, the plurality of nutrients comprises a first plurality of nutrients and a second plurality of nutrients. In certain aspects, the second plurality of nutrients comprises at least one of trace minerals, salts, and vitamins to enhance the composition.

In some embodiments, the plurality of nutrients comprises least one of nutrient selected from the group consisting of alpha-ketoglutaric acid, ACE buffer, potassium hydroxide, phosphate buffer, deionized water, and any combination thereof. In certain embodiments, the plurality of nutrients comprises one or more trace minerals. In various embodiments, the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickle chloride, sodium tungstate, sodium selenite, and any combination thereof.

In other embodiments, the plurality of nutrients comprises one or more salts. In various embodiments, the salts comprise at least one salt selected from the group consisting of potassium chloride, ammonium chloride, sodium hydrogen phosphate, calcium chloride, magnesium sulfate. and any combination thereof.

In yet other embodiments, the plurality of nutrients comprises one or more vitamins. In various embodiments, the vitamins comprise at least one vitamin selected from the group consisting of biotin, folic acid, pyridoxine hydrochloride, riboflavin, thiamine hydrochloride, nicotinic acid, DL-calcium pantothenate, vitamin B₁₂, p-aminobenzoic acid, thiocidic acid, and any combination thereof.

In certain embodiments, the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof. In some embodiments, the Candidatus Liberibacter inoculum is Candidatus Liberibacter asiaticus. In other embodiments, the Candidatus Liberibacter inoculum is Candidatus Liberibacter americanus. In yet other embodiments, the Candidatus Liberibacter inoculum is Candidatus Liberibacter africanus. In other embodiments, the Candidatus Liberibacter inoculum is Candidatus Liberibacter solanacearum.

In certain aspects, the medium is configured to support the growth of the Candidatus Liberibacter inoculum as a culture in a biofilm form.

In some embodiments, the composition is configured to have a pH between about 7 and a pH of about 12. In other embodiments, the composition is configured to have a pH between about 7 and a pH of about 11. In yet other embodiments, the composition is configured to have a pH between about 7 and a pH of about 10. In other embodiments, the composition is configured to have a pH between about 7 and a pH of about 9. In yet other embodiments, the composition is configured to have a pH between about 7 and a pH of about 8.1. In other embodiments, the composition is configured to have a pH between about 7 and a pH of about 8.

In some embodiments, the composition is configured to have an oxygen tension of less than about 30% of air. In other embodiments, the composition is configured to have an oxygen tension of between about 10% of air and about 30% of air. In yet other embodiments, the composition is configured to have an oxygen tension of between about 15% of air and about 25% of air. In other embodiments, the composition is configured to have an oxygen tension of between about 10% of air and about 20% of air. In yet other embodiments, the composition is configured to have an oxygen tension of less than about 10% of air. In other embodiments, the composition is configured to have an oxygen tension of between about 5% of air and about 10% of air. In yet other embodiments, the composition is configured to have an oxygen tension of between about 1% of air and about 10% of air. In other embodiments, the composition is configured to have an oxygen tension of between about 1% of air and about 5% of air.

In another aspect, a method of growing a bacterium is provided. The method comprises the steps of: i) inoculating a composition using a Candidatus Liberibacter bacteria; and ii) physiochemically adjusting the composition to comprise a pH between 7 and 12 and an oxygen tension of less than about 30% of air of the composition.

In some aspects, the method further comprises the step of combining a plurality of nutrients with the composition. In various embodiments, the plurality of nutrients comprises a first plurality of nutrients and a second plurality of nutrients. In certain aspects, the second plurality of nutrients comprises at least one of trace minerals, salts, and vitamins to enhance the composition.

In some embodiments, the plurality of nutrients comprises least one of nutrient selected from the group consisting of alpha-ketoglutaric acid, ACE buffer, potassium hydroxide, phosphate buffer, deionized water, and any combination thereof. In certain embodiments, the plurality of nutrients comprises one or more trace minerals. In various embodiments, the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickle chloride, sodium tungstate, sodium selenite, and any combination thereof.

In other embodiments, the plurality of nutrients comprises one or more salts. In various embodiments, the salts comprise at least one salt selected from the group consisting of potassium chloride, ammonium chloride, sodium hydrogen phosphate, calcium chloride, magnesium sulfate. and any combination thereof.

In yet other embodiments, the plurality of nutrients comprises one or more vitamins. In various embodiments, the vitamins comprise at least one vitamin selected from the group consisting of biotin, folic acid, pyridoxine hydrochloride, riboflavin, thiamine hydrochloride, nicotinic acid, DL-calcium pantothenate, vitamin Biz, p-aminobenzoic acid, thiocidic acid, and any combination thereof.

In various aspects, the method further comprises the step of combining a Candidatus Liberibacter inoculum with the composition. In certain embodiments, the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof. In some embodiments, the Candidatus Liberibacter inoculum is Candidatus Liberibacter asiaticus. In other embodiments, the Candidatus Liberibacter inoculum is Candidatus Liberibacter americanus. In yet other embodiments, the Candidatus Liberibacter inoculum is Candidatus Liberibacter africanus. In other embodiments, the Candidatus Liberibacter inoculum is Candidatus Liberibacter solanacearum.

In some embodiments, the composition is configured as a solidified media with an adjusted neutral pH. In other embodiments, the Candidatus Liberibacter bacteria is derived from at least one bacteria selected from the group consisting of a leaf and one or more infected psyllids.

In yet another aspect, a kit for a bacterial growth medium is provided. The kit comprises a composition comprising a plurality of nutrients, a Candidatus Liberibacter inoculum, and an instruction for combination of the plurality of nutrients and the Candidatus Liberibacter inoculum. In certain embodiments, the composition is configured as a solidified media with an adjusted neutral pH.

In various embodiments, the plurality of nutrients comprises a first plurality of nutrients and a second plurality of nutrients. In certain aspects, the second plurality of nutrients comprises at least one of trace minerals, salts, and vitamins to enhance the composition.

In some embodiments, the plurality of nutrients comprises least one of nutrient selected from the group consisting of alpha-ketoglutaric acid, ACE buffer, potassium hydroxide, phosphate buffer, deionized water, and any combination thereof. In certain embodiments, the plurality of nutrients comprises one or more trace minerals. In various embodiments, the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickle chloride, sodium tungstate, sodium selenite, and any combination thereof.

In other embodiments, the plurality of nutrients comprises one or more salts. In various embodiments, the salts comprise at least one salt selected from the group consisting of potassium chloride, ammonium chloride, sodium hydrogen phosphate, calcium chloride, magnesium sulfate. and any combination thereof.

In yet other embodiments, the plurality of nutrients comprises one or more vitamins. In various embodiments, the vitamins comprise at least one vitamin selected from the group consisting of biotin, folic acid, pyridoxine hydrochloride, riboflavin, thiamine hydrochloride, nicotinic acid, DL-calcium pantothenate, vitamin Biz, p-aminobenzoic acid, thiocidic acid, and any combination thereof.

In certain embodiments, the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof. In some embodiments, the Candidatus Liberibacter inoculum is Candidatus Liberibacter asiaticus. In other embodiments, the Candidatus Liberibacter inoculum is Candidatus Liberibacter americanus. In yet other embodiments, the Candidatus Liberibacter inoculum is Candidatus Liberibacter africanus. In other embodiments, the Candidatus Liberibacter inoculum is Candidatus Liberibacter solanacearum.

In certain aspects, the medium is configured to support the growth of the Candidatus Liberibacter inoculum as a culture in a biofilm form.

In some embodiments, the composition is configured to have a pH between about 7 and a pH of about 12. In other embodiments, the composition is configured to have a pH between about 7 and a pH of about 11. In yet other embodiments, the composition is configured to have a pH between about 7 and a pH of about 10. In other embodiments, the composition is configured to have a pH between about 7 and a pH of about 9. In yet other embodiments, the composition is configured to have a pH between about 7 and a pH of about 8.1. In other embodiments, the composition is configured to have a pH between about 7 and a pH of about 8.

In some embodiments, the composition is configured to have an oxygen tension of less than about 30% of air. In other embodiments, the composition is configured to have an oxygen tension of between about 10% of air and about 30% of air. In yet other embodiments, the composition is configured to have an oxygen tension of between about 15% of air and about 25% of air. In other embodiments, the composition is configured to have an oxygen tension of between about 10% of air and about 20% of air. In yet other embodiments, the composition is configured to have an oxygen tension of less than about 10% of air. In other embodiments, the composition is configured to have an oxygen tension of between about 5% of air and about 10% of air. In yet other embodiments, the composition is configured to have an oxygen tension of between about 1% of air and about 10% of air. In other embodiments, the composition is configured to have an oxygen tension of between about 1% of air and about 5% of air.

In another aspect, a host-free microbial culture is provided. The host-free microbial culture comprises a composition, wherein the composition further comprises: a Candidatus Liberibacter inoculum; a Bacillus species; and one or more antibiotics. In certain embodiments, the one or more antibiotics are not effective to inhibit growth of the Candidatus Liberibacter inoculum.

In some embodiments, the Bacillus species is genetically modified. In other embodiments, the Bacillus species is Bacillus subtilis. In yet other embodiments, the Bacillus species is Bacillus cereus.

In certain embodiments, the one or more antibiotics comprises at least one antibiotic selected from the group consisting of a vancomycin antibiotic, a streptomycin antibiotic, and a polymyxin antibiotic.

In various embodiments, the one or more antibiotics comprises a vancomycin antibiotic. In some embodiments, the vancomycin antibiotic comprises a dose of about 50 μg/ml to about 99 μg/ml. In other embodiments, the vancomycin antibiotic comprises a dose of about 10 μg/ml to about 500 μg/ml. In yet other embodiments, the vancomycin antibiotic comprises a dose of about 50 μg/ml to about 250 μg/ml. In other embodiments, the vancomycin antibiotic comprises a dose of about 50 μg/ml to about 200 μg/ml. In yet other embodiments, the vancomycin antibiotic comprises a dose of at least 100 μg/ml. In other embodiments, the vancomycin antibiotic increases growth abundance of the Candidatus Liberibacter inoculum of at least a 3 fold change. In yet other embodiments, the vancomycin antibiotic increases growth abundance of the Candidatus Liberibacter inoculum of at least a 5 fold change. In other embodiments, the vancomycin antibiotic increases growth abundance of the Candidatus Liberibacter inoculum of at least a 7 fold change. In yet other embodiments, the vancomycin antibiotic increases growth abundance of the Candidatus Liberibacter inoculum of at least a 7.08 fold change. In other embodiments, the vancomycin antibiotic increases growth abundance of the Candidatus Liberibacter inoculum of at least a 8 fold change. In yet other embodiments, the vancomycin antibiotic increases growth abundance of the Candidatus Liberibacter inoculum of at least a 10 fold change. In other embodiments, the vancomycin antibiotic reduces growth abundance of the Candidatus Liberibacter inoculum.

In various embodiments, the one or more antibiotics comprises a streptomycin antibiotic. In some embodiments, the streptomycin antibiotic comprises a dose of about 0.2 μg/ml to about 0.49 μg/ml. In other embodiments, the streptomycin antibiotic comprises a dose of about 0.01 μg/ml to about 10 μg/ml. In yet other embodiments, the streptomycin antibiotic comprises a dose of about 0.1 μg/ml to about 5 μg/ml. In other embodiments, the streptomycin antibiotic comprises a dose of about 0.1 μg/ml to about 2.5 μg/ml. In yet other embodiments, the streptomycin antibiotic comprises a dose of about 0.1 μg/ml to about 1 μg/ml. In other embodiments, the streptomycin antibiotic comprises a dose of at least 0.5 μg/ml. In yet other embodiments, the streptomycin antibiotic increases growth abundance of the Candidatus Liberibacter inoculum of at least a 3 fold change. In other embodiments, the streptomycin antibiotic increases growth abundance of the Candidatus Liberibacter inoculum of at least a 4 fold change. In yet other embodiments, the streptomycin antibiotic increases growth abundance of the Candidatus Liberibacter inoculum of at least a 5 fold change. In other embodiments, the streptomycin antibiotic increases growth abundance of the Candidatus Liberibacter inoculum of at least a 5.41 fold change. In yet other embodiments, the streptomycin antibiotic increases growth abundance of the Candidatus Liberibacter inoculum of at least a 6 fold change. In other embodiments, the streptomycin antibiotic increases growth abundance of the Candidatus Liberibacter inoculum of at least a 8 fold change. In yet other embodiments, the streptomycin antibiotic increases growth abundance of the Candidatus Liberibacter inoculum of at least a 10 fold change. In other embodiments, the streptomycin antibiotic reduces a growth abundance of the Candidatus Liberibacter inoculum.

In various embodiments, the one or more antibiotics comprises a polymyxin antibiotic. In some embodiments, the polymyxin antibiotic comprises a dose level of 0.5 μg/ml to 4 μg/ml. In other embodiments, the polymyxin antibiotic comprises a dose level of 0.1 μg/ml to 10 μg/ml. In other embodiments, the polymyxin antibiotic comprises a dose level of 0.1 μg/ml to 8 μg/ml. In yet other embodiments, the polymyxin antibiotic comprises a dose level of 1 μg/ml to 5 μg/ml. In other embodiments, the polymyxin antibiotic comprises a dose level of 2 μg/ml to 5 μg/ml. In yet other embodiments, the polymyxin antibiotic comprises a dose of about 4 μg/ml. In other embodiments, the polymyxin antibiotic increases growth abundance of the Candidatus Liberibacter inoculum of at least a 2 fold change. In yet other embodiments, the polymyxin antibiotic increases growth abundance of the Candidatus Liberibacter inoculum of at least a 3 fold change. In other embodiments, the polymyxin antibiotic increases growth abundance of the Candidatus Liberibacter inoculum of at least a 3.71 fold change. In yet other embodiments, the polymyxin antibiotic increases growth abundance of the Candidatus Liberibacter inoculum of at least a 4 fold change. In other embodiments, the polymyxin antibiotic increases growth abundance of the Candidatus Liberibacter inoculum of at least a 5 fold change. In yet other embodiments, the polymyxin antibiotic increases growth abundance of the Candidatus Liberibacter inoculum of at least a 8 fold change. In other embodiments, the polymyxin antibiotic increases growth abundance of the Candidatus Liberibacter inoculum of at least a 10 fold change. In yet other embodiments, the polymyxin antibiotic reduces growth abundance of the Candidatus Liberibacter inoculum.

In certain embodiments, growth of the Candidatus Liberibacter in the culture is inversely correlated with the abundance of the Bacillus species in the culture.

In various embodiments, the culture further comprises a plurality of nutrients. In some embodiments, the plurality of nutrients comprises a first plurality of nutrients and a second plurality of nutrients. In other embodiments, the second plurality of nutrients comprises at least one of trace minerals, salts, and vitamins to enhance the composition. In yet other embodiments, the plurality of nutrients comprises least one of nutrient selected from the group consisting of alpha-ketoglutaric acid, ACE buffer, potassium hydroxide, phosphate buffer, deionized water, and any combination thereof. In other embodiments, the plurality of nutrients comprises one or more trace minerals. In yet other embodiments, the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickle chloride, sodium tungstate, sodium selenite, and any combination thereof. In other embodiments, the plurality of nutrients comprises one or more salts. In yet other embodiments, the salts comprise at least one salt selected from the group consisting of potassium chloride, ammonium chloride, sodium hydrogen phosphate, calcium chloride, magnesium sulfate. and any combination thereof. In other embodiments, the plurality of nutrients comprises one or more vitamins. In yet other embodiments, the vitamins comprise at least one vitamin selected from the group consisting of biotin, folic acid, pyridoxine hydrochloride, riboflavin, thiamine hydrochloride, nicotinic acid, DL-calcium pantothenate, vitamin Biz, p-aminobenzoic acid, thiocidic acid, and any combination thereof.

In certain embodiments, the Candidatus Liberibacter inoculum is at least one bacterium selected from the group consisting of Candidatus Liberibacter asiaticus, Candidatus Liberibacter americanus, Candidatus Liberibacter afric anus, Candidatus Liberibacter solanacearum, and any combination thereof. In some embodiments, the Candidatus Liberibacter inoculum is Candidatus Liberibacter asiaticus. In other embodiments, the Candidatus Liberibacter inoculum is Candidatus Liberibacter americanus. In yet other embodiments, the Candidatus Liberibacter inoculum is Candidatus Liberibacter africanus. In other embodiments, the Candidatus Liberibacter inoculum is Candidatus Liberibacter solanacearum.

In yet another aspect, a method of growing a host-free microbial culture is provided. The method comprises the steps of: inoculating a composition with a Candidatus Liberibacter inoculum; combining a Bacillus species and the composition; and treating the Candidatus Liberibacter inoculum with one or more antibiotics, wherein the one or more antibiotics are not effective to inhibit growth of the Candidatus Liberibacter inoculum.

In another aspect, a method of growing a host-free microbial culture is provided. The method comprises the steps of: inoculating a composition with a Candidatus Liberibacter inoculum; combining a Bacillus species and the composition; and treating the Candidatus Liberibacter inoculum with one or more antibiotics, wherein the one or more antibiotics are effective to inhibit growth of the Candidatus Liberibacter inoculum. The previously described embodiments of the host-free microbial culture are applicable to the method of growing a host-free microbial culture described herein.

In yet another aspect, an assay for inhibiting growth of a Candidatus Liberibacter inoculum in a host-free microbial culture is provided. The assay comprises the steps of i) inoculating a composition with a Candidatus Liberibacter inoculum; ii) combining the composition and a Bacillus species; and iii) treating the Candidatus Liberibacter inoculum with one or more antibiotics, whereby the one or more antibiotics inhibit growth of the Candidatus Liberibacter inoculum in the host-free microbial culture. The previously described embodiments of the host-free microbial culture are applicable to the assay for inhibiting growth of a Candidatus Liberibacter inoculum in a host-free microbial culture described herein.

In another aspect, an assay for growing a Candidatus Liberibacter inoculum in a host-free microbial culture is provided. The assay comprises the steps of i) inoculating a composition with a Candidatus Liberibacter inoculum; ii) combining the composition and a Bacillus species with; and iii) treating the Candidatus Liberibacter inoculum with one or more antibiotics, wherein the one or more antibiotics do not inhibit growth of the Candidatus Liberibacter inoculum in the host-free microbial culture. The previously described embodiments of the host-free microbial culture are applicable to the assay for growing a Candidatus Liberibacter inoculum in a host-free microbial culture described herein.

In yet another aspect, a kit for inhibiting growth of a Candidatus Liberibacter inoculum is provided. The kit comprises a host-free microbial culture, a Candidatus Liberibacter inoculum, a Bacillus species, one or more antibiotics, and an instruction for combination. The previously described embodiments of the host-free microbial culture are applicable to the kit for inhibiting growth of a Candidatus Liberibacter inoculum described herein.

In another aspect, an assay for detecting a HLB infection in a plant is provided. The assay comprises the steps of: i) obtaining a tissue from the plant; ii) incubating the tissue in a culture; and iii) quantifying DNA of a bacteria in the cultured tissue, whereby a quantity of the bacterial DNA in the cultured tissue indicates the HLB infection in the plant.

In some embodiments, the culture comprises basal PBS. In various embodiments, the culture comprises one or more of Na₂HPO₄, KH₂PO₄, KCl, NaCl, CaCl₂, MgCl₂, and any combination thereof.

In certain embodiments, the tissue is a leaf. In some aspects, the leaf is a leaf disc. In other aspects, the leaf disc is cut from a disc midrib.

In various embodiments, the quantification of DNA is performed via a PCR assay. Types of PCR assays are well known to the skilled artisan and can be performed according to knowledge in the art.

In various embodiments, the PCR assay utilizes a primer. In certain embodiments, the quantification of the DNA is performed after incubation of the tissue for 3 days.

In yet another aspect, a kit for detecting a HLB infection in a plant is provided. The kit comprises a culture and an instruction for incubating a tissue in the culture.

In some embodiments, the culture comprises basal PBS. In various embodiments, the culture comprises one or more of Na₂HPO₄, KH₂PO₄, KCl, NaCl, CaCl₂, MgCl₂, and any combination thereof.

In certain embodiments, the tissue is a leaf. In some aspects, the leaf is a leaf disc. In other aspects, the leaf disc is cut from a disc midrib.

### EXAMPLES

### Example 1

### Preparation of Materials and Methods For Host-Free Culture

### Membrane Biofilm Reactor: Construction and Operation

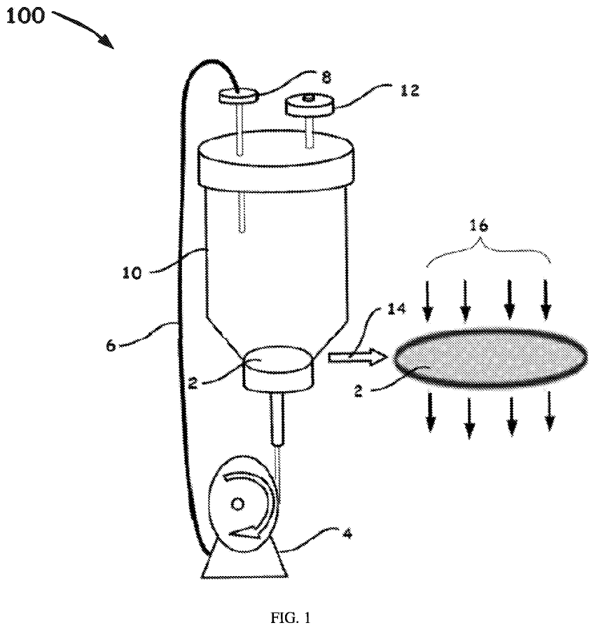

Biofilms were grown using a custom-built membrane biofilm reactor (MBR), such as the example MBR system 100 shown in FIG. 1. The reactor was built using a filter funnel from Millipore (Millipore, USA) and a polyvinylidene fluoride membrane filter with a pore size of 0.1 m (Millipore). The reactor and tubing were sterilized by autoclaving, and the filter membrane was sterilized by exposure to UV radiation (27 Id, 30 min/side). After sterilization, the membrane was placed and secured in the funnel. Next, 150 ml of medium was pumped into the filter funnel. The reactor funnel was then inoculated with inoculum containing ‘C. L. asiaticus’. Biofilms were grown for 10-15 days at room temperature under the sterilized hood.

FIG. 1 illustrates generally, as referenced by the numeral 100, a non-limiting membrane biofilm reactor (MBR) system that can be utilized herein to culture Candidatus Liberibacter pathogens (e.g., ‘C. L. asiaticus’). An exemplary means of developing ‘C. L. asiatisus’ biofilms was to innoculate with extract from midribs of leaves and stems of ‘C. L. asiatisus’-infected citrus plants. It is understood that while the MBR 100 system shown in FIG. 1 is used to aid in understanding the invention, other configurations and components may also be used without departing from the scope and spirit of the invention. As examples, the MBR system 100, as utilized herein, can grow cells or tissues and is an engineered/designed system that can use aerobic or anaerobic processes were warranted to support a biologically active environment to include organisms or biologically active substances originated from such organisms.

Thus, FIG. 1 shows a housing 10 for housing a membrane 2, and optionally also any and all membrane supporting structure(s) (not shown), a pump 4, a line 6 (e.g. a feed line) from the pump 4 coupled to a syringe filter 8, and a gas inlet/gas filter 12 for admitting a gas to the vessel. As also shown in FIG. 1 the arrow denoted by the reference numeral 14, illustrates the membrane within the housing 10 having a directional flow (as denoted by directional arrows 16, of the medium through the biofilm for illustrative purposes. The housing 10 is sterilisable, and the membrane-supporting structure may be removable from the vessel or the housing. A sterilisable housing may be useful for preventing contamination of the biolayer, the membrane or of other portions of the membrane biofilm reactor (MBR) system 100. The membrane reactor (MBR) system 100 often has means for removing solidified media from the membrane. The solid matter may be for example a product of the bioreactor or it may be a portion of the biolayer to include a scraper, a blower or some other suitable means.

The membrane 2 is often substantially planar although not constrained to such a design. The membrane 2 can be configured as a nanoporous, mesoporous, macroporous or may or combinations thereof with corresponding nanoscale and/or mesoscale and/or microscale pores. The membrane 2 with other materials, such as, a woven or non-woven fibrous material or a non-fibrous porous material. The support material (not shown) may hydrophilic or hydrophobic and be of a knitted material a polymer, an inorganic material, etc. The support material may have a nanoporous solid or gel therein and/or thereon. The membrane 2 may be capable of separating a gas at the gas face from a nutrient solution at the nutrient face is capable of allowing diffusion of a nutrient solution. Appropriate pathogen(s) cells and nutrients (first and second nutrients, as disclosed herein) as well as configured physiological adjustments are utilized to enable the proper growth of the disclosed embodiments.

### Medium Composition and Preparation

The medium for ‘C. L. asiaticus’ biofilm culture was based on BM7 medium, which is used to isolate and grow L. crescens, with several modifications: first, the nutrient strength was lowered, to make a nutrient-poor environment; second, the buffering capacity was increased; and third, the medium was enhanced with a complex mixture of salts, vitamins and trace minerals. Medium preparation and composition are detailed in Table 1 and Table 2 as follows:

TABLE 1
Composition of growth medium (1 L) for ‘C.
L. asiatisus’ biofilm culture.

| NAME | AMOUNT | |
|---|---|---|
| Alpha-Ketoglularic acid | 2 | g |
| ACE buffer | 10 | g |
| KOH | 3.75 | g |
| Sall mixture (100X) | 10 | ml |
| Phosphate buffer 1M (pH 7.0) | 10 | mL |
| DI water | To 880 | mL |
| Adjust the pH of above solution to 7.0 and autoclave at | | |
| 121° C. After cooling, aseptically add: | | |
| Fetal bovine serum | 25 | mL |
| Hink's TNM-FH insect medium | 75 | mL |
| Vitamin mixture (100X) | 10 | mL |
| Trace mineral mixtures (100X) | 10 | mL |

TABLE 2
Composition of salt mixture, trace minerals and vitamin mixtures
used in ‘C. L. asiatisus’ growth medium.

| | Concentration |
|---|---|
| 100x Salt mixture | (g/L) |
| KCl | 38.0 |
| NH₄Cl | 20.0 |
| NaH₂PO₄ · H₂O | 6.9 |
| CaCl₂ · 2H₂O | 4.0 |
| MgSO₄ · 7H₂O | 20.0 |

| 100x Trace mineral mixture | g/L |
|---|---|
| Nitrilotriacetic acid (NTA) | 1.500 |
| - pH NTA solution to 10, then add: | |
| MnCl₂ · 4H₂O | 0.100 |
| FeSO₄ · 7H₂O | 0.300 |
| CoCl₂ · 6H₂O | 0.170 |
| ZnCl₂ | 0.100 |
| CuSO₄ · 5H₂O | 0.040 |
| AlK(SO₄)₂ · 12H₂O | 0.005 |
| H₃BO₃ | 0.005 |
| Na₂MoO₄ | 0.090 |
| NiCl₂ | 0.120 |
| NaWO₄ · 2H₂O | 0.020 |
| Na₂SeO₄ | 0.100 |
| - pH solution to 7.0 | |
| 100x Vitamin mix | mg/L |
| Biotin | 2.0 |
| Folic acid | 2.0 |
| Pyridoxine hydrochloride | 10.0 |
| Riboflavin | 5.0 |
| Thiamine hydrochloride | 5.0 |
| Nicotinic acid | 5.0 |
| DL-Calcium pantothenate | 5.0 |
| Vitamin B₁₂ | 0.1 |
| p-Aminobenzoic acid | 5.0 |
| Thiocidic (lipoic) acid | 5.0 |

### Inoculum Preparation

The biofilm culture of ‘C. L. asiaticus’ was initiated from citrus plant extracts. The ‘C. L. asiaticus’-infected Hamlin sweet orange samples were provided by collaborators at the University of Florida Citrus Research and Education Center. Upon arrival, the samples were used for inoculum preparation right away or kept at 4° C. for not more than 2 days until use. The leaves and stems were first washed with running distillery water to remove all large particles, followed by a surface disinfection procedure. Surface disinfection was carried out in a clean hood equipped with laminar flow by immerging leaves and stems in 10% bleach, 5% bleach and 75% ethanol. Each step was conducted for 10 min. After this, the leaves and stems were rinsed several times with autoclaved distillery water to remove all traces of bleach and ethanol. Mid-leaves were collected using sterilized razor blades to remove the leaf part. Mid-leaves and stems were cut in small pieces (˜0.5 cm) on sterilized petri dishes before being blended with mortar and pestle. Blended midribs and stems were collected in a sterilized Falcon tube with 10 mL of fresh medium. The mixture was vortexed vigorously at maximum speed for 5 min before being centrifuged at 2,500 g/5 min. The supernatant was carefully collected in another sterilized Falcon tube and used as an inoculum for membrane biofilm reactors (MBRs).

After the initial biofilm culture of ‘C. L. asiaticus’ was established (10 days of operation), new sequential biofilm cultures were generated by transferring a small fraction of a previous biofilm culture to sterile MBRs with fresh medium (˜1% inoculated volume). Control reactors were operated using either extracts from ‘C. L. asiaticus’-free citrus plants or autoclaved biofilm culture as the inoculum.

### Biofilm Culture Sampling

Biofilm cultures from MBRs were sampled at the initial inoculation and at the end of each transfer. At initial inoculation, the inoculum was mixed well with the medium and drawn through the sampling outlet. Samples were aliquoted and kept at −20° C. for further examination. At the end of each transfer (10-15 days), the upper part of the planktonic culture was gently poured into the sterilized container. Next, the filter funnel was disassembled. Membranes harboring the biofilm were carefully transferred to the petri dishes using sterilized tweezers. All these steps were carried out aseptically. Biofilm parts could be cut in portions for further experiments such as SEM or genomic DNA extraction. Otherwise, biofilm was mixed with planktonic culture and homogenized by vigorous vortexing.

To determine the growth of ‘C. L. asiaticus’ in MBRs over time, several MBRs were operated identically under the same conditions. The biofilm culture from these MBRs was then sampled sequentially at different points in time.

Genomic DNA Extraction, PCR and qPCR

Genomic DNA was extracted from either initial citrus plants or biofilm culture samples. Plant DNA was extracted using a Plant Easy Kit (Qiagen) following the manufacturer's procedure. Community genomic DNA from biofilm culture samples was extracted using a manual extraction method as described earlier.

The conventional primers specific to 16S rRNA fragments of ‘C. L. asiaticus’ (O11/O12c) were used to determine the presence of ‘C. L. asiaticus’ in the extracted gDNA samples using PCR. The PCR reaction was conducted as described previously.

Primer probes specific to 16S rRNA fragments of ‘C. L. asiaticus’(forward primer, Las1R; reverse primer, Las1F:) were designed, in order to quantify the 16S rRNA gene of ‘C. L. asiaticus’ in the gDNA samples. Both of the primer sequences are specific to ‘C. L. asiaticus’ and amplified the fragment of 140 bp from its 16S rRNA gene. The qPCR reactions were performed using Step One qPCR (Applied Bioscience). A standard curve was created using pCR4-TOPO plasmids inserted with the 140 bp fragment amplified from ‘C. L. asiaticus’ using Las1R and Las1F. Since each genome of ‘C. L. asiaticus’ contains 3 copies of the 16S rRNA gene, the result was converted to the number of ‘C. L. asiaticus’ genomic equivalents.

### Cloning and Sequencing

To confirm the DNA fragments amplified by O11/O12c (1160 bp) were specific to ‘C. Liberibacter asiaticus’, the bands were excised and purified on agarose gel after PCR and cloned them into the pCR4-TOPO plasmids, followed by transformation into One Shot™ TOP10 competent Escherichia coli cells (Invitrogen™). Plasmids containing the insert were sent for sequencing (WSU, USA). Sequences were analyzed using MEGA version 7 and compared with those in the BLAST GenBank. Sequences were deposited into the NICBI GenBank under submission number SUB4578636.

### Community Analysis

The V1-V3 variable region of the 16S rRNA gene was amplified from the extracted gDNA with primers 27F (GAGTTTGATCMTGGCTCAG) and 515R (TTACCGCGGCTGCTGGCAC) (Kroes et al., 1999). Paired barcodes were added to each primer for further sorting of samples from the pool after sequencing. Amplicons were prepared using PCRs. Each PCR reaction was performed in duplicate 50-μL reactions containing 50-100 ng of DNA, 1× Phusion HF Buffer, 0.2 μM of each barcoded primer (IDT), 10 μM of dNTPs and 1.00 unit of Phusion® High Fidelity DNA Polymerase (Thermo Scientific, USA). PCR was performed using a Mastercycler® thermal cycler (Eppendorf, NY, USA) under the following conditions: (i) an initial denaturation step at 98° C. for 30 seconds, (ii) 30 amplification cycles (98° C. for 10 seconds, 57° C. for 30 seconds and 72° C. for 30 seconds), and (iii) a final extension at 72° C. for 5 minutes. After this PCR amplification, the amplicons were purified (Qiagen PCR purification kit) and quantified (Qubit). Barcoded amplicons were pooled and sequenced using a PacBio-RSII sequencer (Washington State University, Pullman, Wash.). PacBio FASTAQ formatted circular consensus sequences were processed and analyzed using mothur v.1.39.

Sequences were quality trimmed using a sliding window of 10 bp and an average quality score of 30, and sequences that contained one or more ambiguous bases or were shorter than 450 bp were removed. Filtered sequences were dereplicated and aligned to a SILVA-based reference alignment (silva.nr_v132.align). The sequences were then screened to remove those that did not align to positions 1044-11892 of the reference alignment, filtered to remove non-informative columns, preclustered to >99.5% identity, and dereplicated. Chimeras were identified and removed using UCHIME (Edgar et al., 2011) as implemented in mothur v.1.39 in self-referential mode. Filtered sequences were classified against the SILVA (v132) reference taxonomies using a naive Bayesian classifier implemented within mothur with an 80% bootstrap cutoff, and sequences that could not be placed within any of the domains of life were removed using remove.lineage (taxon=unknown). Sequences were clustered into operational taxonomic units (OTUs) at 0.03 average distances using the average neighbor algorithm in mothur. OTUs were classified based upon the sequence classifications described above.

### SEM Imaging

To image the cell morphology and cells attached on biofilm samples, scanning electron micrographs were taken of biofilm forming after culturing. Filter membrane with biofilms formed on it was used for further treatment and viewed with SEM. Samples were fixed with fixative solution containing 2.0% gluteraldehyde, 2.0% paraformaldehyde in 0.1 M PBS, pH 7.2 for up to 12 h at 4° C., then washed 3 times in 0.1 M phosphate buffer saline (pH 7.2) for 10 min each, dehydrated further in ethanol solutions of 10%, 35%, 50%, 75%, 95% and 100% for 10 min each. The dehydration in 100% ethanol (200 proof) was done 3 times. The samples were immediately immersed twice for 10 min in hexamethyldisilazane (SigmaAldrich, MO, USA) followed by air-drying for 9 h under a hood. The samples were sputter-coated with gold and were imaged with a Quanta SEM (FEI, TX, USA).

### Example 2

### Presence and Growth of Ca. L. asiaticus in Biofilm Cultures

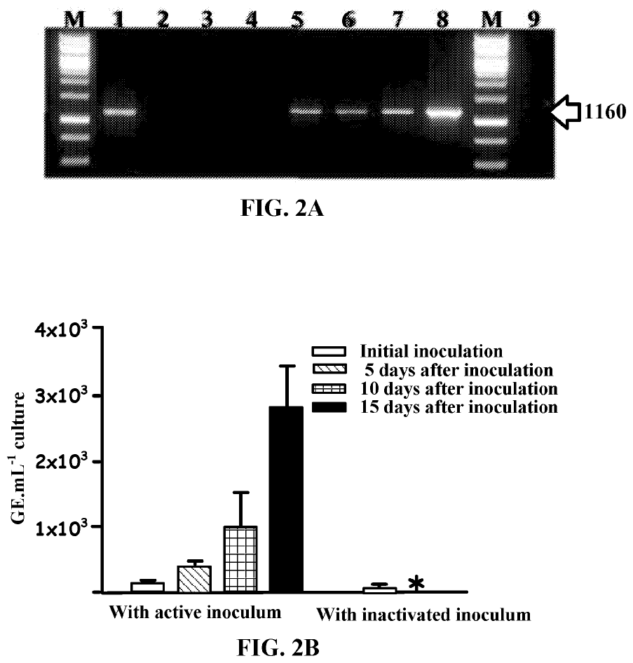

FIG. 2A and FIG. 2B show validation of the presence and growth of “Ca. L. asiaticus” in biofilm cultures. Biofilm were cultured in the MBRs without aeration and at pH 7.0 FIG. 2A in particular shows PCR amplification of a 1160 bp fragment (also denoted by the directional arrow) of the “Ca. L. asiaticus” 16S rDNA gene using specific primers O11 and O12c. Tracks marked M contained a 1-kb ladder; tracks 1 and 8: infected citrus Hamlin; track 2: initial inoculation with inactivated inoculum; track 3: 15 days after inoculation with inactivated inoculum; track 4: initial inoculation with 1% of active inoculum from 2^{nd} transfer; track 5: biofilm 10 days after inoculation with active inoculum; track 6: planktonic culture 10 days after inoculation with active inoculum; track 7: mixture of biofilm and planktonic culture 10 days after inoculation with active inoculum; track 9: water. FIG. 2B shows validation and quantification of “Ca. L. asiaticus” growth in the MBR at the 5^{th} transfer using qPCR with a probe specific for the 16S rDNA of “Ca. L. asiaticus”. The error bars represent the standard error of the mean of replicated experiments (n=4) and an asterisk (*) indicates no detectable signal in qPCR.

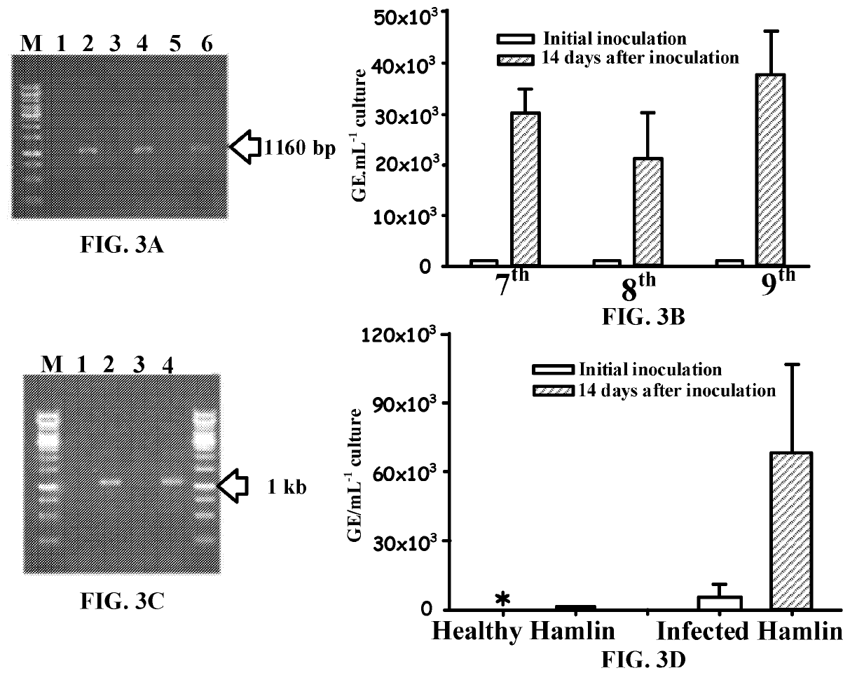

FIG. 3A, FIG. 3B, FIG. 3C, and FIG. 3D illustrate validation of the presence and growth of ‘C. L. asiaticus’ in biofilm cultures over many transfers and independent repetitions. FIG. 3A and FIG. 3B particularly show validation of ‘C. L. asiaticus’ presence and growth in the MBR at the₇^{th}, 8^{th} and 9^{th} transfers with the PCR using conventional specific primers for ‘C. L. asiaticus’ (O11 and O12c) and qPCR with a specific probe for 16S rRNA of ‘C. L. asiaticus’. Tracks 1, 3, 5: initial inoculation; tracks 2. 4, 6: 14 days after inoculation; track M: 1-kb marker.

FIG. 3C and FIG. 3B show validation of ‘C. L. asiaticus’ presence and growth in a newly repeated MBR experiment at a different laboratory. The increase of ‘C. L. asiaticus’ genome equivalents in an MBR inoculated with a plant extract from ‘C. L. asiaticus’-infected Hamlin over 14 days of operation clearly demonstrates the growth of ‘C. L. asiaticus’ in a biofilm reactor. Tracks 2 and 4: initial inoculation; tracks 1 and 3: 14 days after inoculation. As before, the error bars represent the standard error of the mean of replicated experiments (n=3) and an asterisk (*) indicates no detectable signal in qPCR.

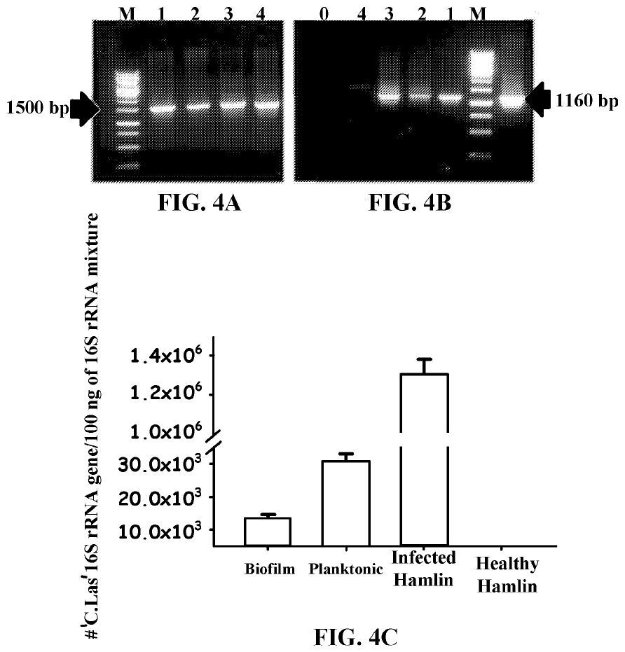

FIG. 4A, FIG. 4B, and FIG. 4C show validation and quantification of the presence of ‘C. L. asiaticus’ 16S rRNA gene among the total 16S rRNA genes amplified from the biofilm cultures. FIG. 4A shows the total of about 1500 bp (denoted with directional arrow) 16S rRNA gene fragments from microbial communities were amplified using universal primer 27F/1492r. FIG. 4B shows These 16S rRNA gene fragments were purified and used as the template for the PCR reactions using ‘C. L. asiaticus’-specific primers (O11/O12c). The specific band of 1160 bp (also denoted with directional arrow) was amplified from the total 16S rRNA gene fragments of biofilm cultures and ‘C. L. asiaticus’-infected citrus Hamlin but not from a mock community which did not contain ‘C. L. asiaticus’. The data suggest the presence of the ‘C. L. asiaticus’ 16S rRNA gene in the total 16S rRNA genes isolated from the microbial community of biofilm cultures. Track 0: water; tracks 1 and 2: planktonic and biofilm from MBR at the 3^{rd} transfer, respectively; track 3: HLB-symptomatic Hamlin; track 4: mock community formed by mixing cultures of S. aureus and A. baumannii; track C: positive control, ‘C. L. asiaticus’-infected citrus. FIG. 4C shows validation and quantification of ‘C. L. asiaticus’ 16S rRNA gene in total 16S rRNA genes isolated from the microbial community of biofilm cultures using qPCR with a specific probe for 16S rRNA of ‘C. L. asiaticus’. Again, the error bars represent the standard error of the mean of replicated experiments (n=3) and an asterisk (*) indicates no detectable signal in qPCR.

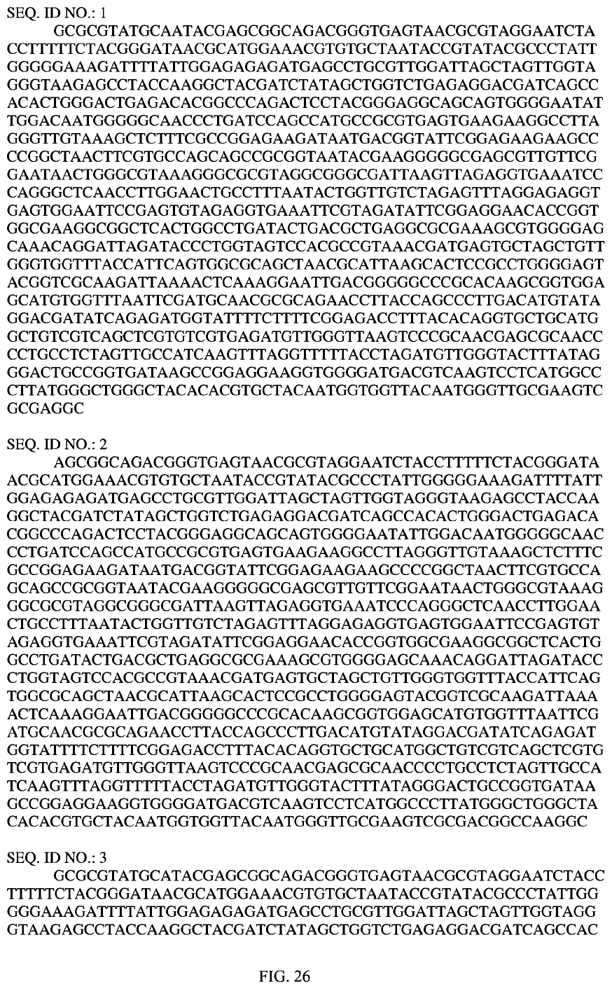

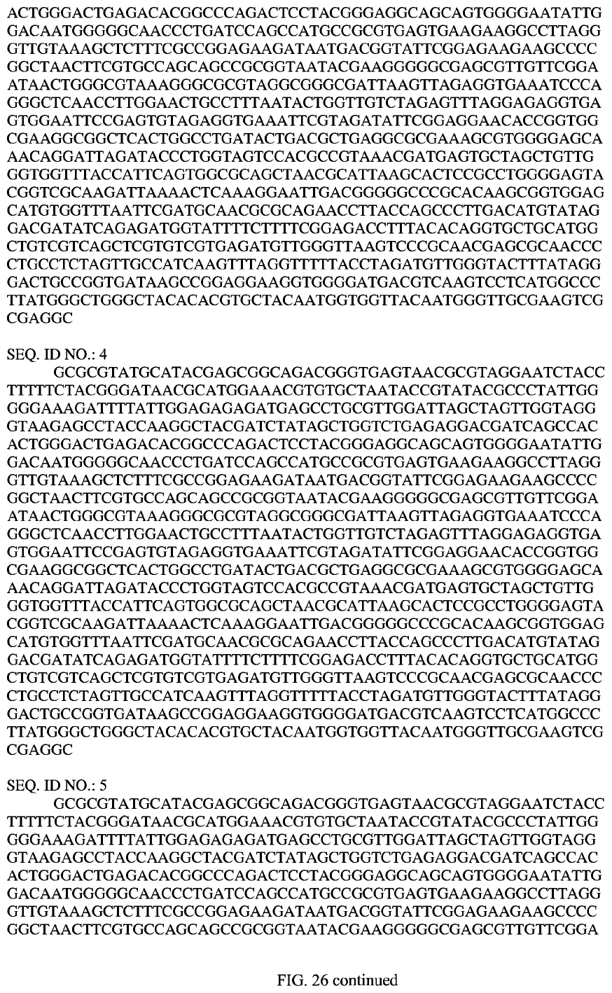

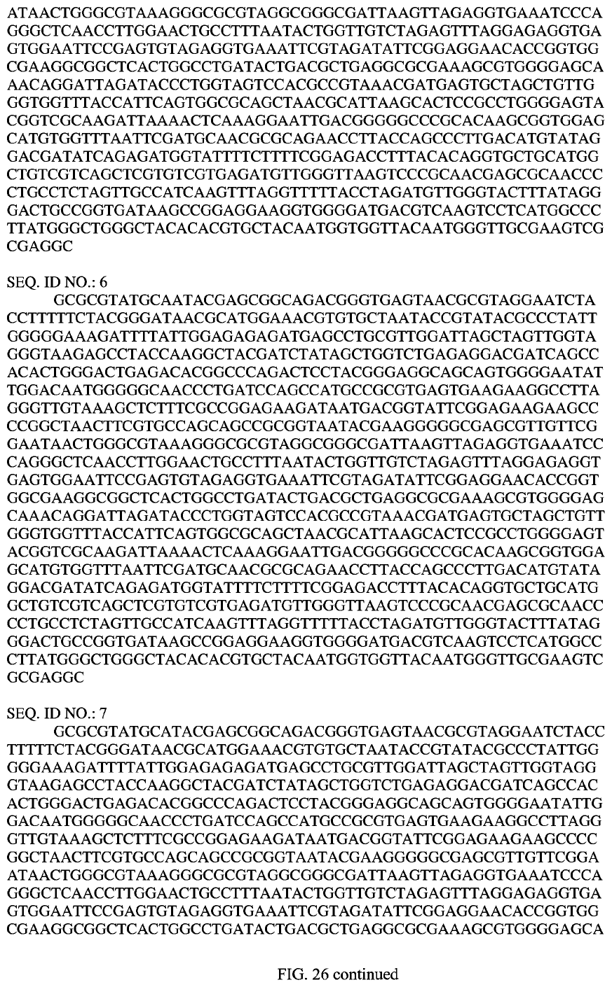

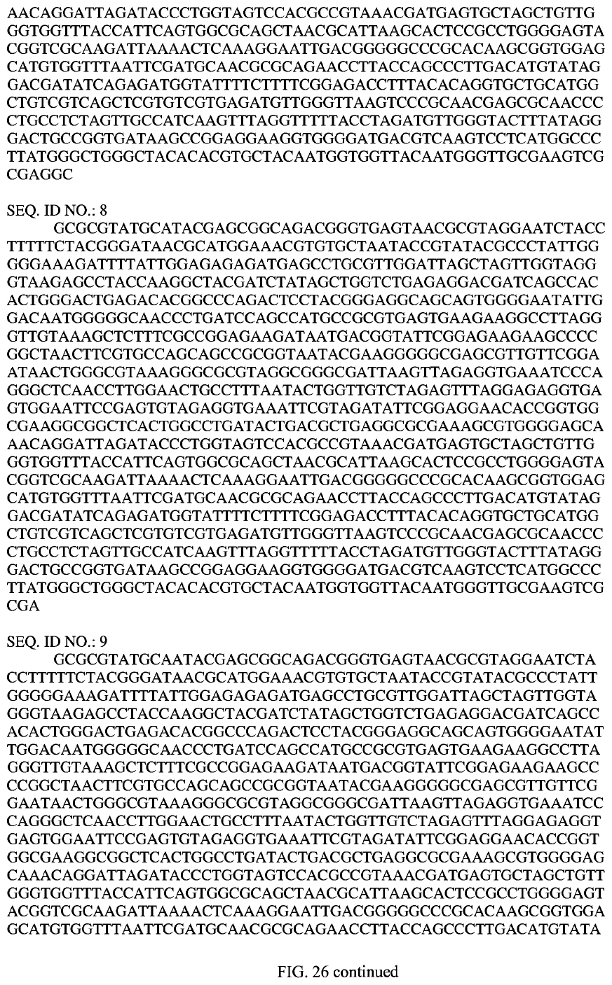

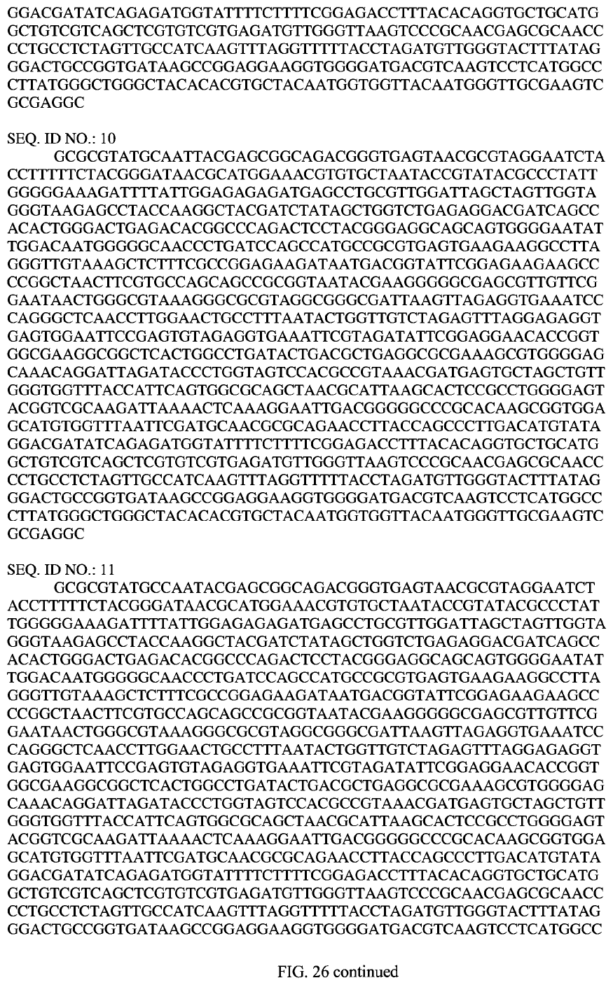

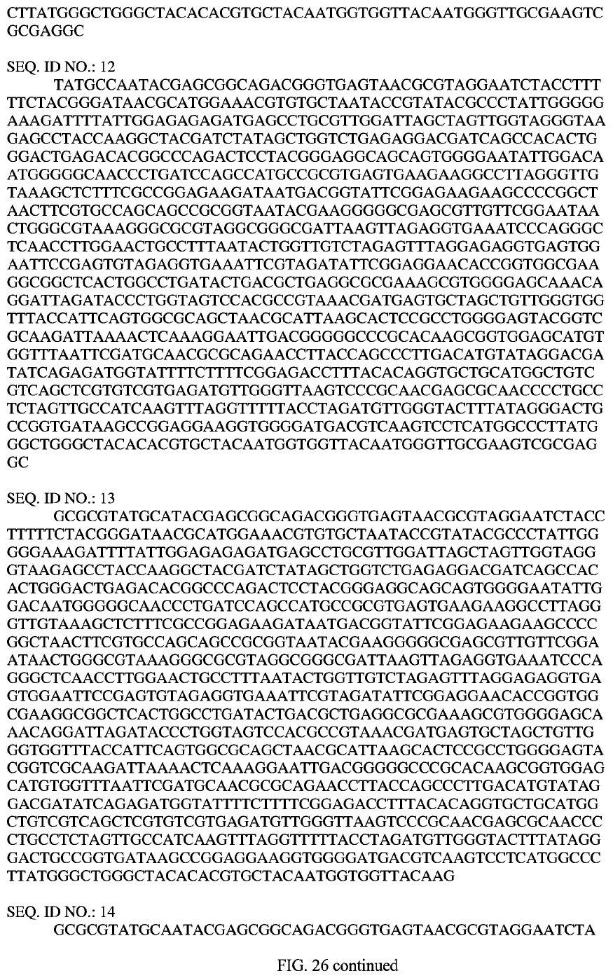

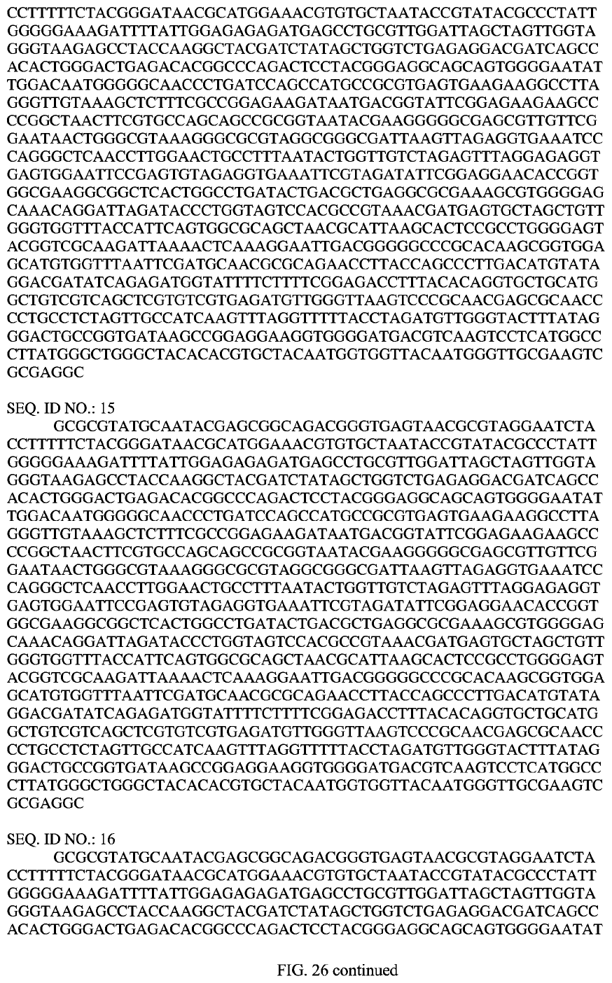

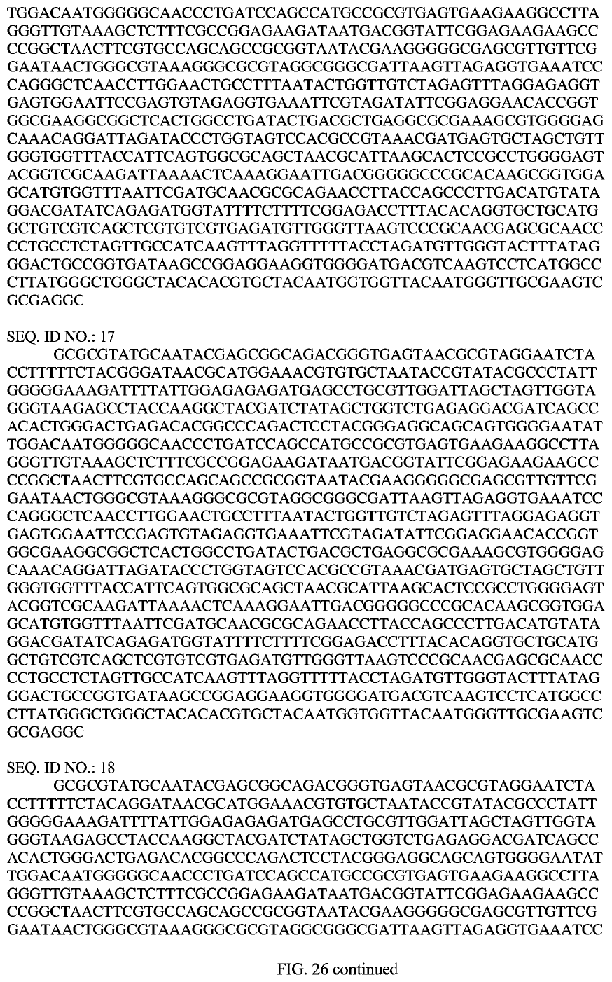

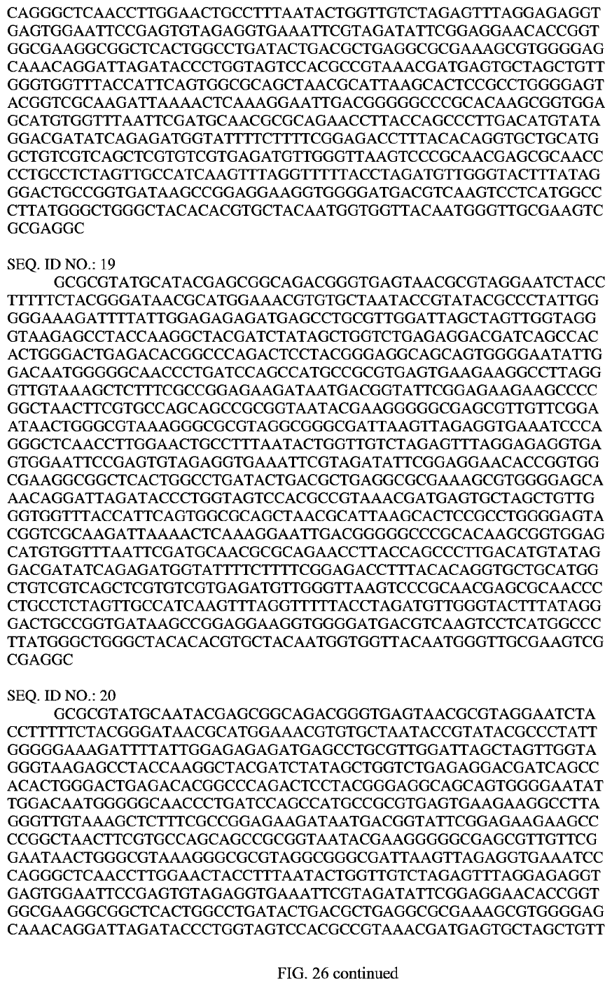

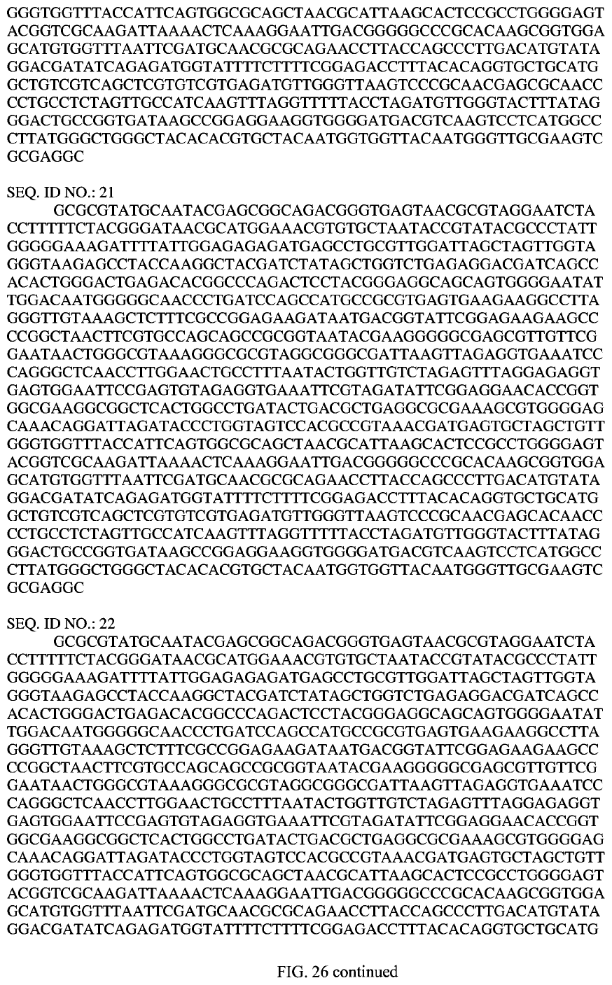

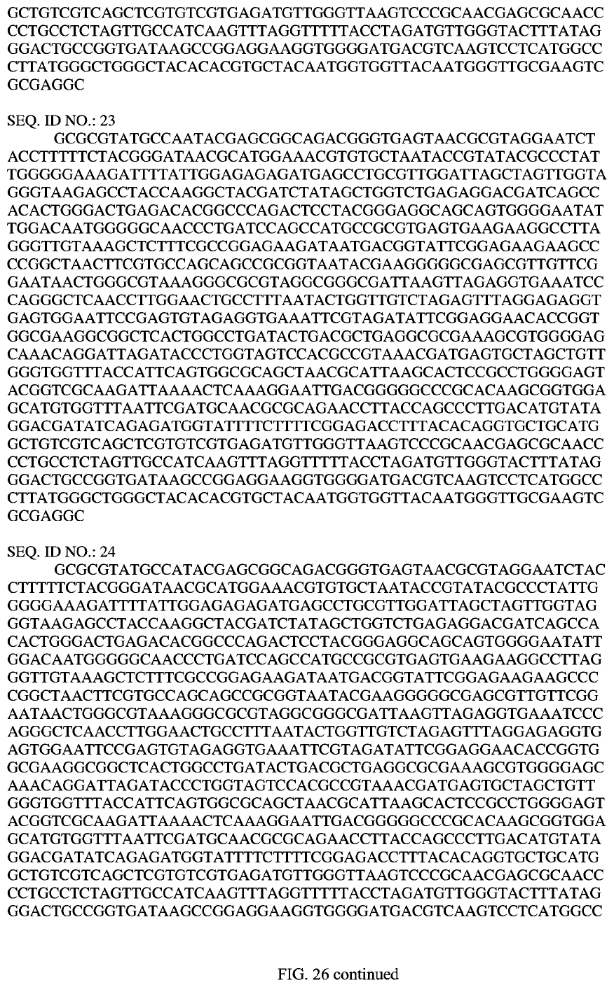

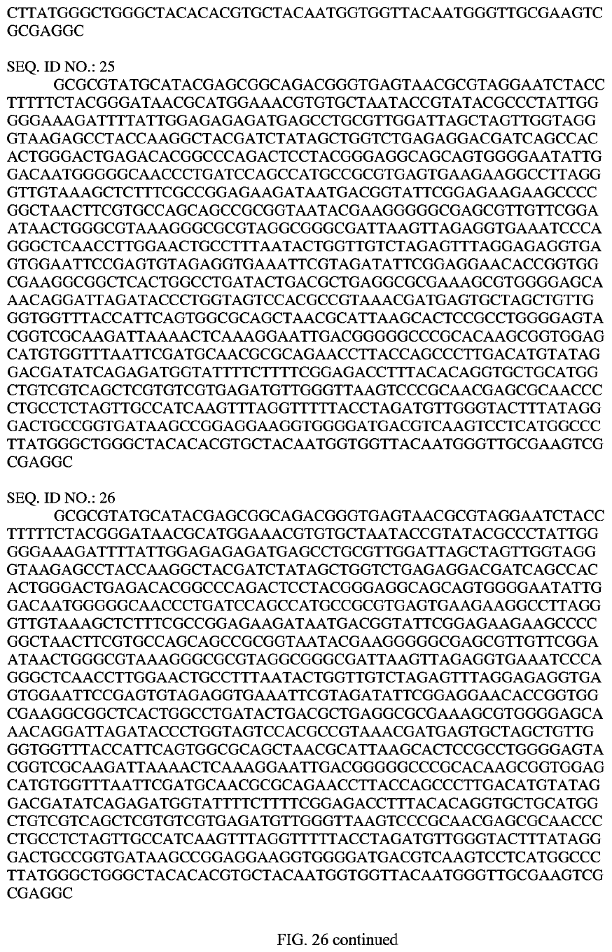

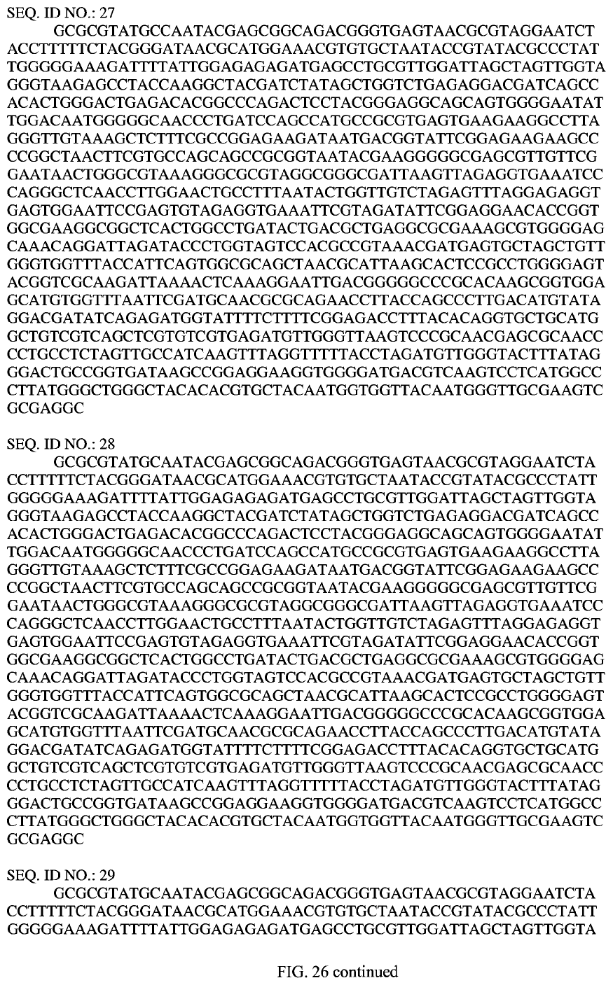

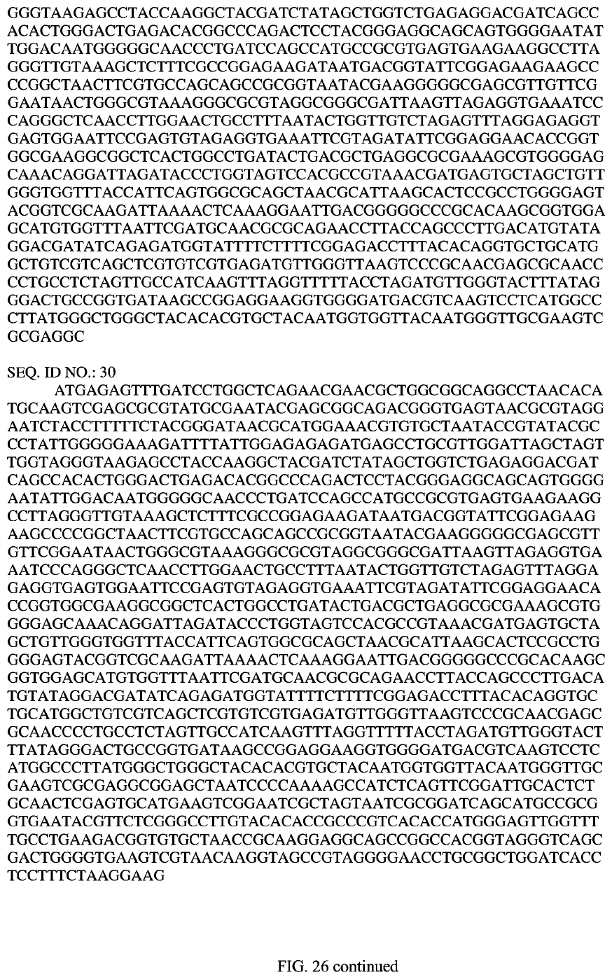

As indicated below, alignment of 16S rRNA gene sequences generated from 29 colonies which were from the biofilms by PCR amplification (1160 bp fragment) using O11/O12c primer. The 29 sequences (SEQ ID NOS: 1-29) were generated from Sanger sequencing at WSU genomics core. The reference sequence (SEQ ID NO: 30) was from Candidatus Liberibacter asiaticus str. psy62, complete genome (GenBank: CP001677.5). The 16S ribosomal RNA of SEQ ID NO: 30 is in the region 854295-855801 with full length of 1507 bp. An alignment of SEQ ID NOS: 1-29 with SEQ ID NO: 30 was performed by Clustal Omega (Multiple Sequence Alignment) program from The European Bioinformatics Institute (EMBL-EBI) and displayed in FIG. 26.

Moreover, some sequences were originally generated via the sequencing procedure (i.e., “raw data”) and were thereafter manually corrected to result in various sequences presented herein. The raw data may contain some noises due to the sequencing machine. As a result, the noises were corrected by comparison with the reference sequence. From the comparisons, the nucleotides that were different from those on the reference sequence were verified to determine if they could be noise identified on the chromatography for correction.

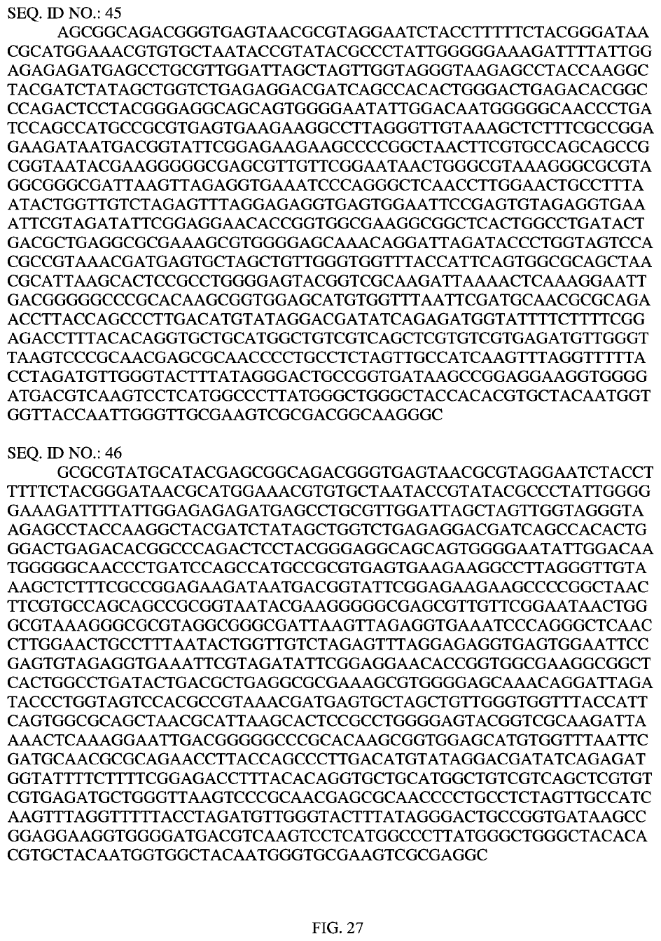

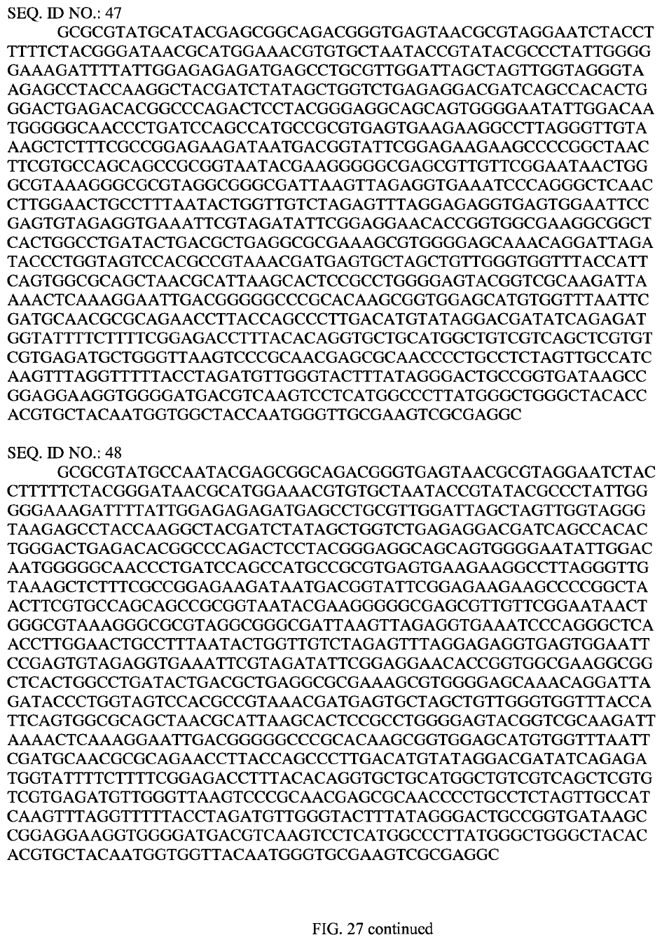

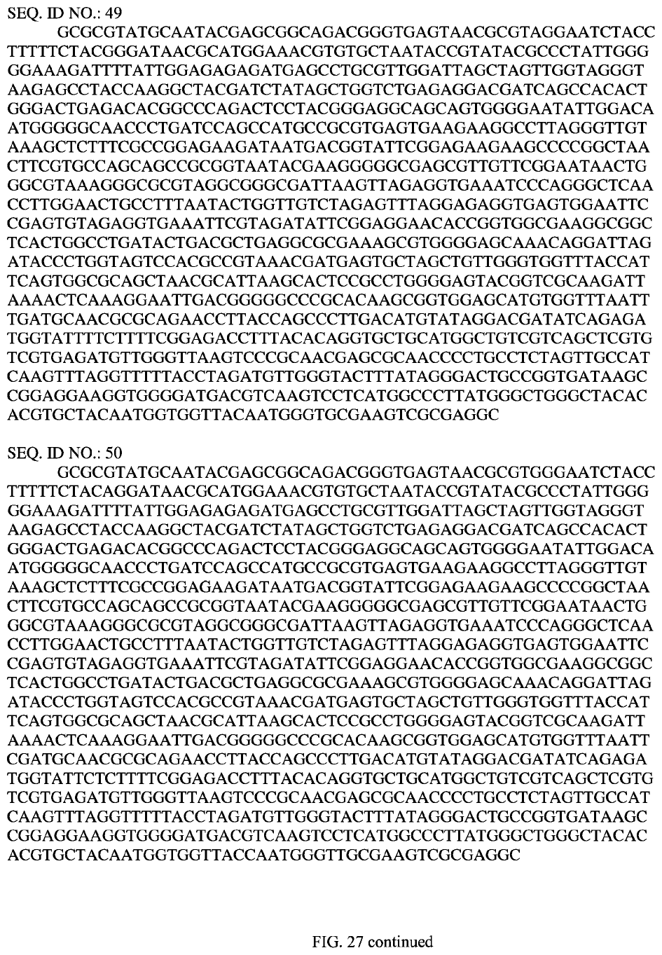

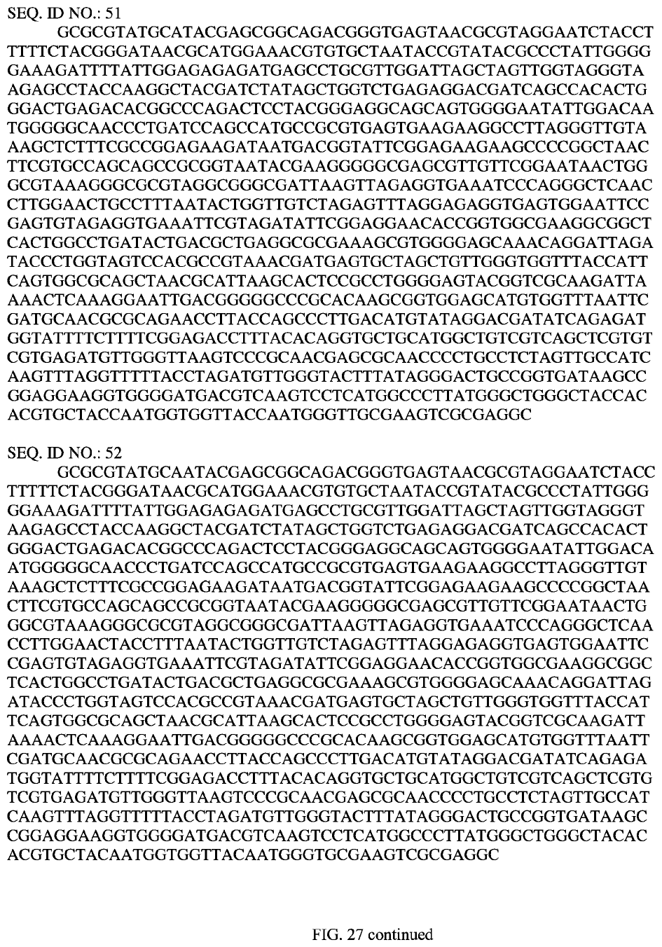

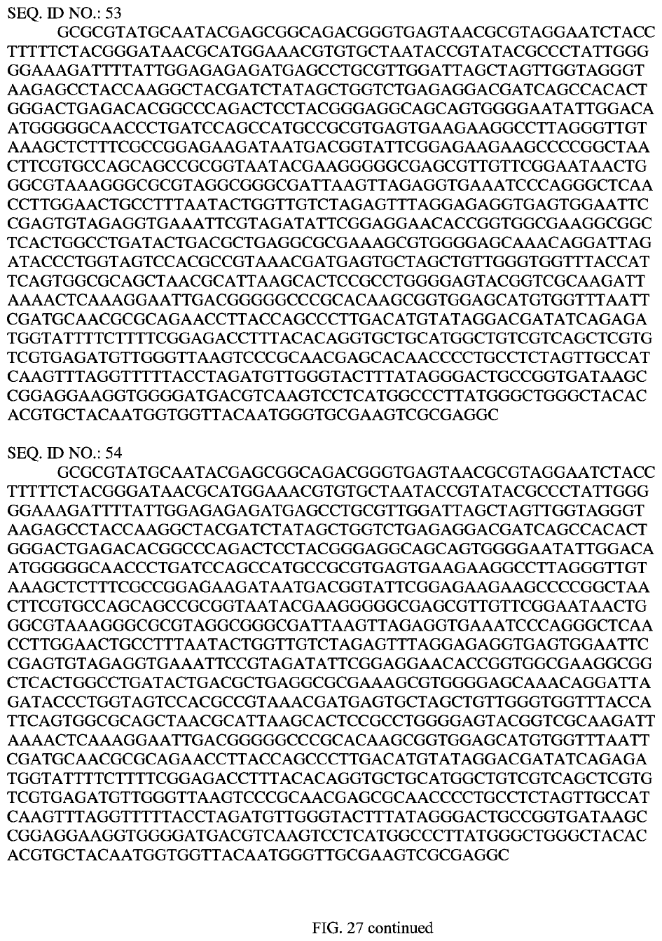

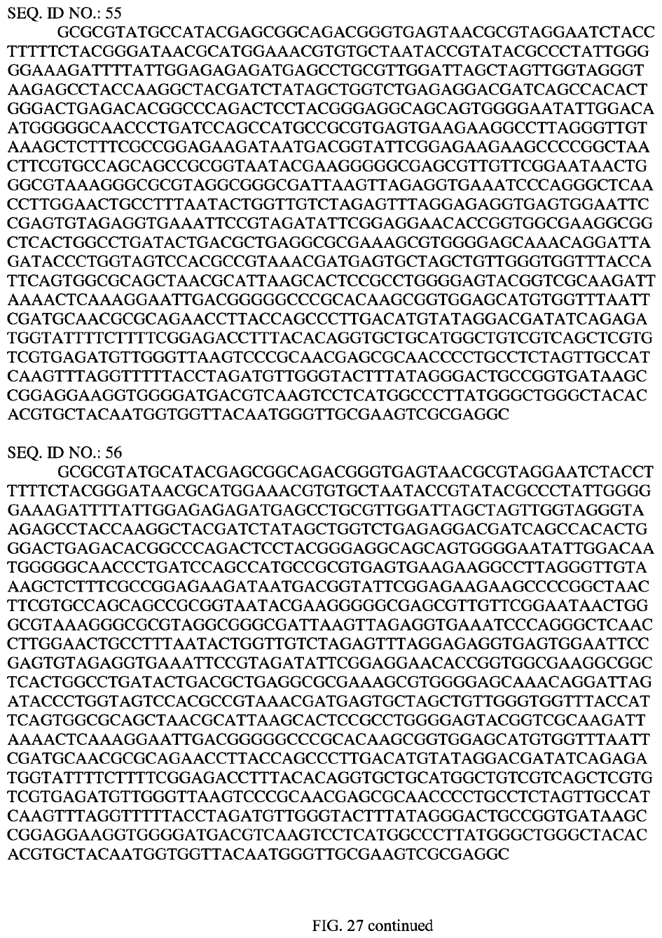

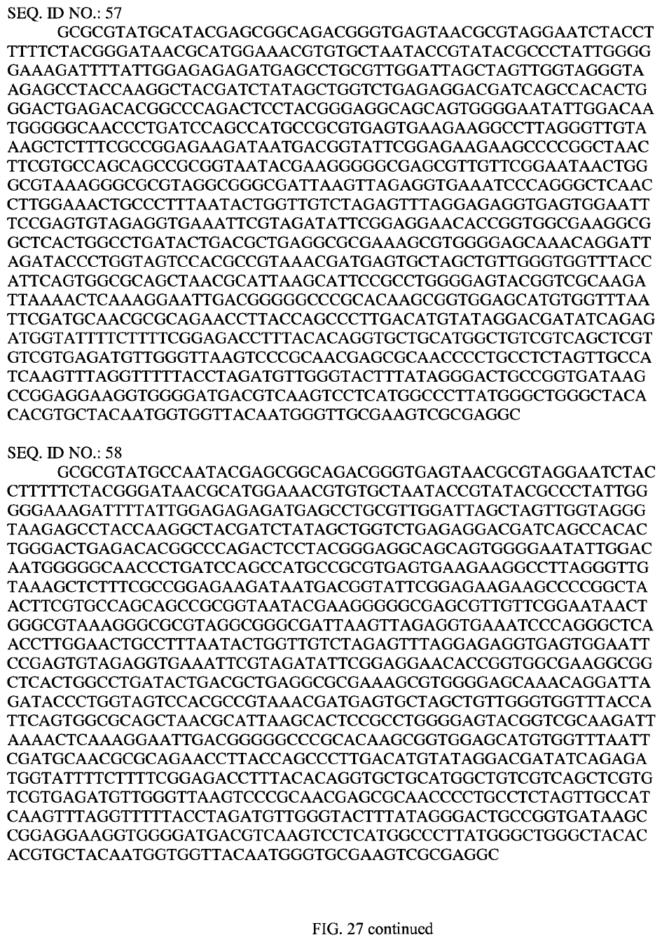

For example, Table 8 identifies the originally generated sequences and their corresponding manually corrected sequences. These originally generated sequences are shown in FIG. 27.

TABLE 8

| Originally Generated Sequence | Manually Corrected Sequence |
|---|---|
| SEQ ID NO: 45 | SEQ ID NO: 2 |
| SEQ ID NO: 46 | SEQ ID NO: 5 |
| SEQ ID NO: 47 | SEQ ID NO: 7 |
| SEQ ID NO: 48 | SEQ ID NO: 11 |
| SEQ ID NO: 49 | SEQ ID NO: 15 |
| SEQ ID NO: 50 | SEQ ID NO: 18 |
| SEQ ID NO: 51 | SEQ ID NO: 19 |
| SEQ ID NO: 52 | SEQ ID NO: 20 |
| SEQ ID NO: 53 | SEQ ID NO: 21 |
| SEQ ID NO: 54 | SEQ ID NO: 22 |
| SEQ ID NO: 55 | SEQ ID NO: 24 |
| SEQ ID NO: 56 | SEQ ID NO: 25 |
| SEQ ID NO: 57 | SEQ ID NO: 26 |
| SEQ ID NO: 58 | SEQ ID NO: 27 |

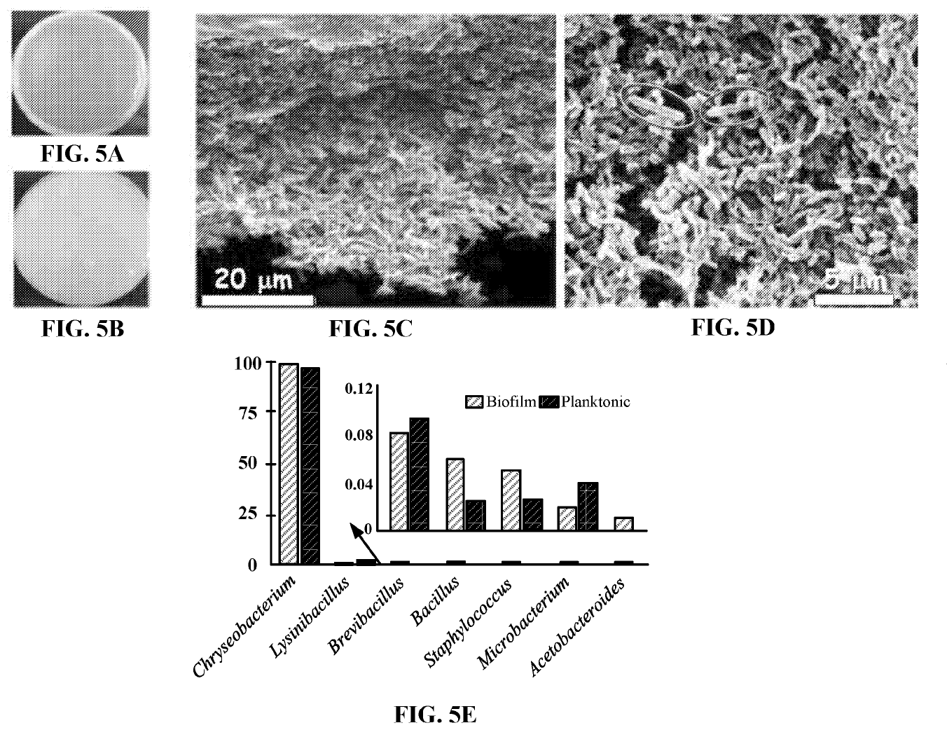

In using an example MBR system 100, as shown in FIG. 1, FIG. 5A, FIG. 5B, FIG. 5C, FIG. 5D, and FIG. 5E illustrate the evident growth of a particular Candidatus Liberibacter pathogen, i.e., “Ca. L. asiaticus” despite still being a minor member of the biofilm community. FIG. 5A thus shows Ca. L. asiaticus”-containing biofilms 52 (orange color in actuality). FIG. 5B shows inactivated inoculum 56 which led to accumulation of a white layer of protein precipitate instead. FIG. 5C shows a scanning electron micrographs of “Ca. L. asiaticus”-containing biofilms to clearly illustrate the presence of multiple microorganisms embedded within extracellular polymeric substance. FIG. 5D shows longer, rod-shaped bacteria (denoted within ellipses for convenience) with the same morphology as L. crescens so as to illustrate an ease of detection as a minor biofilm component. FIG. 5 shows microbial community profiles of “Ca. L. asiaticus”-containing biofilm and planktonic culture revealed that circa 99% of the population of the cultures, both biofilm and planktonic, was a Chryseobacterium species.

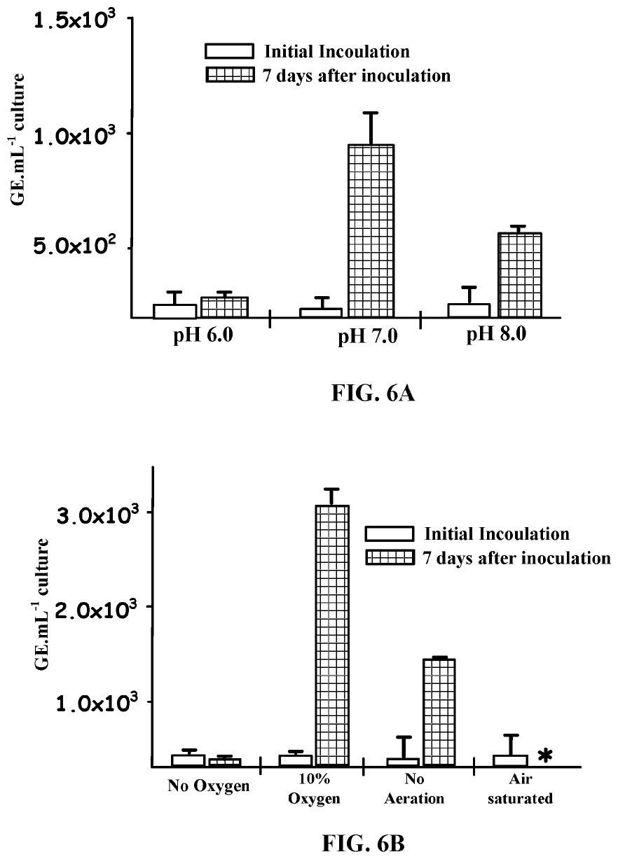

FIG. 6A and FIG. 6B are obtained plots of pH and oxygen tension on the growth of “Ca. L. asiaticus” in a mixed culture with other biofilm microorganisms. FIG. 6A indicates no significant growth was detected at pH 6, but there was growth at pH 7 and 8. All cultures were grown without aeration FIG. 6BS shows higher genome equivalent levels of “Ca. L. asiaticus” were measured in the cultures grown under 10% oxygen tension than at other oxygen tensions. No growth was detected in the culture lacking oxygen or in the culture with an oxygen-saturated condition. All cultures were grown at pH 7. Again, the error bars represent the standard error of the mean of replicated experiments (n=3) and an (*) asterisk indicates no detectable signal in qPCR.

### Example 3

### Preparation of Materials and Methods for Host-Free Co-Cultures

Establishment of Host-Free Mixed Microbial Cultures of “Ca. L. asiaticus” from ACP In Vitro

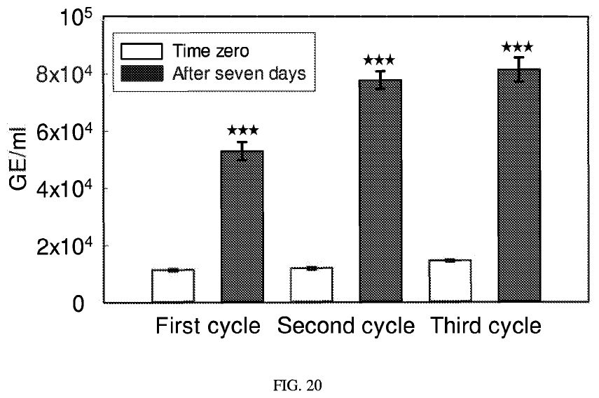

FIG. 20 shows the presence and growth of “Ca. L. asiaticus” in the culture during two growth cycles. The “first cycle” represents “Ca. L. asiaticus” growth when the homogenate “Ca. L. asiaticus”-infected ACP was used as the inoculum, and the “second cycle” represents “Ca. L. asiaticus” growth when the 7-day-old culture from the first cycle was used as the inoculum. In the third cycle, the 7-day-old culture from the second cycle was used as the inoculum. Cloning and sequencing of the 1,160-bp band from the PCR reaction confirmed the specificity of the “Ca. L. asiaticus” assay (100% identity with the 16S rDNA gene fragment from “Ca. L. asiaticus strain psy62”, 4/4 sequences). The qPCR results show that the genome equivalents (GE) of “Ca. L. asiaticus” increased from 11,928±448 at the time of inoculation to 65,839±3166 after 7 days of incubation. No significant difference was observed between the second and third cycles. This is the first time that “Ca. L. asiaticus”-infected ACP has been used as a source of inoculum to establish a host-free mixed microbial culture of “Ca. L. asiaticus.”

### Example 4

### Effects of Antibiotics on “Ca. L. asiaticus” Growth in Microbial Cultures

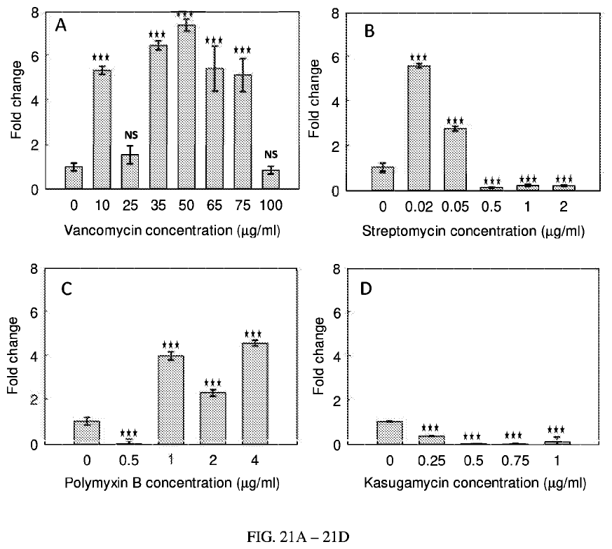

The largest changes in “Ca. L. asiaticus” GE occurred in cultures to which 50 μg/ml of vancomycin (7.35±0.27 (±SD) fold) (FIG. 21A), 0.02 μg/ml of streptomycin (5.56±0.15 fold) (FIG. 21B), or 4 μg/ml of polymyxin B (4.54±0.83 fold) (FIG. 21C) were added. Treatment with kasugamycin did not enhance “Ca. L. asiaticus” growth. However, when the concentration of streptomycin was increased to >0.5 μg/ml, “Ca. L. asiaticus” growth was reduced compared to that in untreated cultures. Treatment of the cultures with 2 or 4 μg/ml of polymyxin B increased “Ca. L. asiaticus” growth 2.28±0.15 or 4.54±0.83 fold, respectively, whereas treatment with <1 μg/ml was correlated with decreased growth compared to that of untreated cultures (0.03±0.13 fold) (FIG. 21C). Overall, it was observed that microbial community modification using antibiotics generally influenced “Ca. L. asiaticus” abundance in a dose-dependent manner.

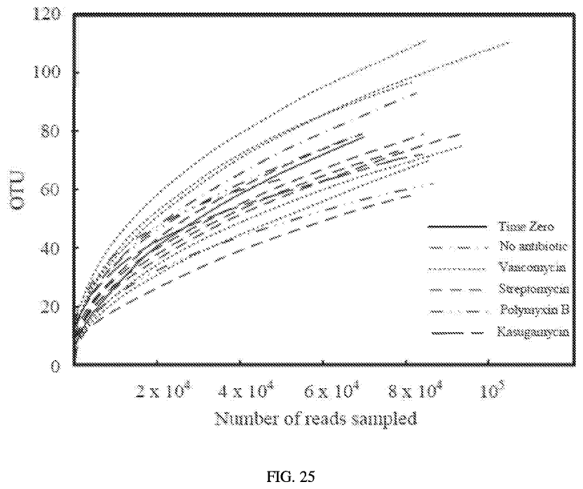

The microbial composition of the host-free mixed microbial cultures was assessed using 16S DNA sequencing after antibiotic treatment. The 16S raw reads obtained from Illumina sequencing were filtered, and 2,159,566 sequences remained. These clustered into a total of 992 operational taxonomic units (OTUs) with 97% sequence identity across all samples. The number of reads ranged between 50,448 and 104,287 with the lowest number belonging to the sample treated with 2 μg/ml polymyxin B and the highest number belonging to the sample treated with 25 μg/ml vancomycin. The small ratio (0.001) of OTUs to the number of reads sampled for both antibiotic-treated and untreated “Ca. L. asiaticus” host-free mixed microbial cultures illustrates that almost all OTUs were identified in these experiments (see FIG. 25). FIG. 25, in particular shows species (OTU) accumulation curves of antibiotic-treated and untreated “Ca. L. asiaticus” host-free mixed cultures. Such samples, as shown in FIG. 25, were treated with 25, 35, 50, 75, or 100 μg/ml of vancomycin; 0.02, 0.05, 0.5, 1 or 2 μg/ml of streptomycin; 0.5, 2, or 4 μg/ml of polymyxin B; 0.25 μg/ml of kasugamycin; or no antibiotic and the original inoculum were analyzed (total of 16 samples). Only one replicate per condition was sequenced.

Chao and inverse Simpson indices were calculated for each sample as shown in Table 3 below, which shows bacterial diversity indices of “Ca. L. asiaticus” mixed cultures. Van: vancomycin; Str: streptomycin; Pmb: polymyxin B; Kag: kasugamycin.

TABLE 3

| Sample | Chao | Invsimpson |
|---|---|---|
| Time zero | 117.79 | 2.64 |
| No antibiotic | 135.33 | 4.08 |
| van 25 μg/ml | 136.76 | 3.18 |
| van 35 μg/ml | 122.26 | 3.20 |
| van 50 μg/ml | 152.84 | 3.92 |
| van 75 μg/ml | 101.45 | 4.20 |
| van 100 μg/ml | 145.92 | 2.70 |
| str 0.02 μg/ml | 92.82 | 1.12 |
| str 0.05 μg/ml | 114 | 1.73 |
| str 0.5 μg/ml | 114.09 | 3.57 |
| str 1 μg/ml | 100.31 | 2.00 |
| str 2 μg/ml | 111.89 | 1.88 |
| pmb 0.5 μg/ml | 83.79 | 1.85 |
| pmb 2 μg/ml | 80.05 | 2.47 |
| pmb 4 μg/ml | 122.30 | 2.61 |
| Kag 0.25 μg/ml | 97.21 | 2.51 |

The Chao index represents the number of distinct 16S sequences that were identified for each condition with an adjustment for the proportion of singletons. The inverse Simpson's index reflects diversity based on the contributions of both the richness and the evenness of all distinct species. When both of these metrics were examined relative to treatment, there were no dose-dependent relationships (see Table 3). In most cases, the treatments showed a weak negative slope (less diversity with more antibiotic), but the variance explained by the linear models (i.e., r²) was between 3.8% and 12.5% (see Table 3). Furthermore, the Chao index and the inverse Simpson's index for the treatment giving the highest fold increase in “Ca. L. asiaticus” (FIG. 20) were not notably different from (higher or lower than) the values for other treatment conditions. That is, changes in overall diversity were not predictive of “Ca. L. asiaticus” growth.

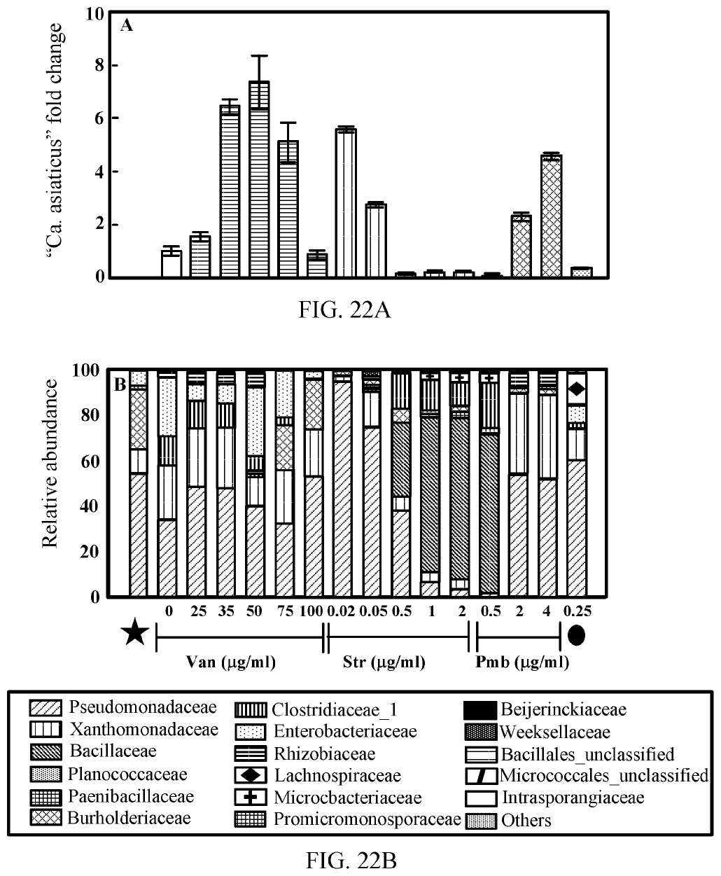

To understand better how community composition changed with each antibiotic treatment, comparisons were made at the family level (FIGS. 22A-22B). Host-free mixed microbial cultures treated with 0.5, 1, or 2 μg/ml of streptomycin or 0.5 μg/ml of polymyxin B had the lowest relatedness to other host-free mixed cultures. In these host-free microbial mixed cultures, the Bacillaceae family was the dominant family in the community. For samples treated with 0.5, 1 or 2 μg/ml streptomycin or 0.5 μg/ml polymyxin B, 32.3%, 69%, 71.7%, or 70.7% of the reads, respectively, were Bacillaceae members. Most of these (more than 99.97%) are classified as Bacillus cereus (98.81% identity with the 16S rDNA fragment of Bacillus cereus as shown in Table 4 below. Table 4, in particular, shows the 10 most dominant OTUs, with the corresponding species and percent identity.

TABLE 4

| | | | | | Accession |
|---|---|---|---|---|---|
| OTU | % Read | Genus | Species | Identity | number |
| 1 | 0.358 | Pseudomonas | Pseudomonas | 98.81% | MN_263205 |
| | | | putida | | |
| 2 | 0.202 | Stenotrophomonas | Stenotrophomonas- | 98.02% | NR_157765 |
| | | | bentonitica | | |
| 3 | 0.140 | Bacillus | Bacillus cereus | 98.81% | NR_113266 |
| 4 | 0.051 | Lysinibacillus | Lysinibacillus | 98.81% | NR_118146 |
| | | | mangiferihumi | | |
| 5 | 0.044 | Clostridium_{—}sensu_{—}stricto_{—}13 | Clostridium | 99.21% | NR_029232 |
| | | | argentinense | | |
| 6 | 0.032 | Burkholderiaceae | Comamonas | 99.6% | NR_113597 |
| | | | terrigena | | |
| 7 | 0.030 | Enterobacteriaceae | Pantoea cypripedii | 98.81% | NR_118394 |
| 8 | 0.021 | Paenibacillus | Paenibacillus | 99.21% | NR_156836 |
| | | | silvae | | |
| 9 | 0.016 | Brevibacillus | Brevibacillus | 99.21% | NR_148616 |
| | | | gelatini | | |
| 10 | 0.015 | Enterobacteriaceae | Flavobacterium | 98.81% | NR_104962 |
| | | | acidificum | | |

For most other antibiotic treatments, the members of Pseudomonadaceae were the most dominant in microbial cultures, with portions ranging from 32.17% to 94.64% (FIGS. 22A-22B). The majority of these Pseudomonadaceae members (95% of reads) are classified as Pseudomonas putida (98.81% identity with the 16S rDNA fragment of Pseudomonas putida (see Table 4). It should be noted that in no-antibiotic control sample, up to 34.18% of the reads were contributed to Pseudomonadaceae while only 0.11% of reads were classified as Bacillaceae members. Thus, the treatment of 1 or 2 μg/ml of streptomycin or 0.5 μg/ml of polymyxin B decreased the proportion of Pseudomonadaceae member (5.5; 9.8; 30.5 times compared to non-antibiotic samples, respectively) (FIGS. 22A-22B), but increase the portion of Bacillaceae. Interestingly, in these treatments “Ca. L. asiaticus” fold change was also reduced significantly compared to those observed for other antibiotic treatments and the no-antibiotic control (FIGS. 22A-22B).

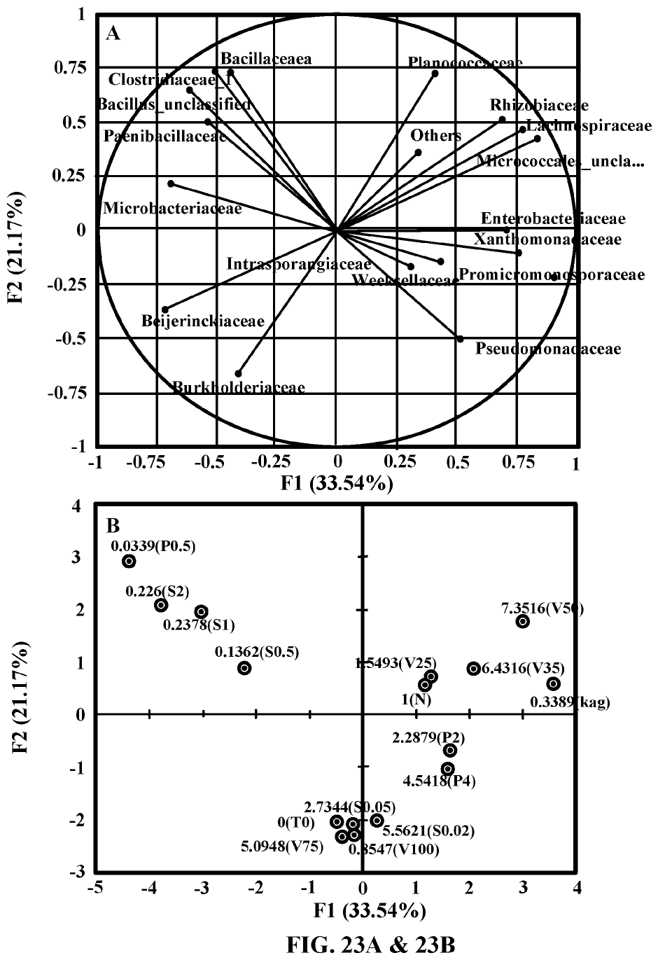

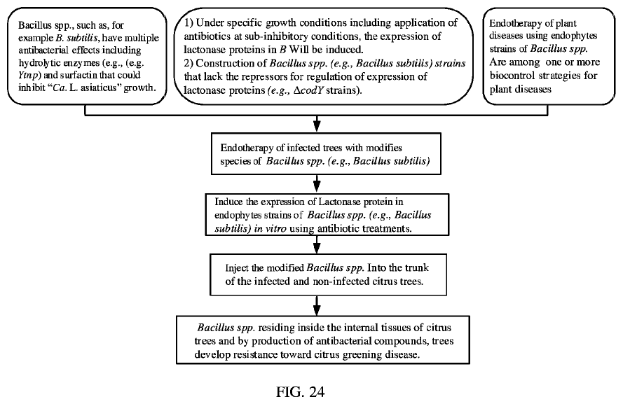

Community composition were compared across treatments using principal component analysis. The first two components explained 54.7% of the variance (FIGS. 23A-23B). The highest fold increases in “Ca. L. asiaticus” (FIG. 22A) were most positively correlated with the presence of bacteria from the Pseudomonadaceae (more than 95% of OTU-1), Xanthomonadaceae (more than 99% of OTU-2), Weeksellaceae (100% of OTU-23), and Enterobacteriaceae (more than 98% of OTU-7) families (FIG. 23A). OTU-1 was identified as Pseudomonas putida with 98.81% similarity, OTU-2 was identified as Stenotrophomonas bentonitica with 98.02% similarity, OTU-23 was identified as Chryseobacterium geocarposphaerae with 99.60% similarity, and OTU-7 was identified as Pantoea cypripedii with 98.81% similarity. The lowest fold increases in “Ca. L. asiaticus” were associated with the presence of Bacillaceae (more than 99% of OTU-3), Clostridiaceae_1 (more than 97% of OTU-5), Bacillales_unclassified (more than 90% of OTU-25), and Paenibacillaceae (more than 99% of OTU-9) families (FIG. 23B). OTU-3 was identified as Bacillus cereus with 98.81% similarity, OTU-25 was identified as Bacillus wiedmannii with 98.81% similarity, and OTU-9 was identified as Brevibacillus gelatini with 99.21% similarity. The samples treated with 0.5, 1 or 2 μg/ml streptomycin or 0.5 μg/ml polymyxin B grouped together in a score plot (FIG. 23B), which is consistent with the high proportion of Bacillaceae family sequences from these libraries (particularly Bacillus cereus).

The 10 most dominant OTUs and the corresponding species and the proportions of these OTUs after the various treatments are presented in Table 5 below. Table 5 in particular shows the proportions of the 10 most dominant OTUs under the various antibiotic treatments. The highlighted cells show the OTUs with a proportion of more than 0.1. TO: Time zero; NA: No antibiotic; Van: vancomycin; Str: streptomycin; Pmb: polymyxin B; Kag: kasugamycin.

TABLE 5

| | | | | Van | Van | Van | Van | Str | Str | Str | Str | Str | Pmb | Pmb | Pmb | Kag |
|---|---|---|---|---|---|---|---|---|---|---|---|---|---|---|---|---|
| OTU | T0 | NA | Van25 | 35 | 50 | 75 | 100 | 0.02 | 0.05 | 0.5 | 1.00 | 2.00 | 0.5 | 2 | 4 | 0.25 |
| 1 | 0.538 | 0.337 | 0.479 | 0.469 | 0.398 | 0.318 | 0.527 | 0.945 | 0.742 | 0.380 | 0.062 | 0.035 | 0.011 | 0.516 | 0.486 | 0.599 |
| 2 | 0.110 | 0.236 | 0.255 | 0.272 | 0.134 | 0.242 | 0.209 | 0.027 | 0.157 | 0.062 | 0.044 | 0.046 | 0.001 | 0.365 | 0.379 | 0.136 |
| 3 | 0.000 | 0.001 | 0.002 | 0.002 | 0.006 | 0.000 | 0.000 | 0.000 | 0.023 | 0.323 | 0.690 | 0.717 | 0.707 | 0.001 | 0.000 | 0.001 |
| 4 | 0.000 | 0.001 | 0.001 | 0.002 | 0.004 | 0.000 | 0.000 | 0.000 | 0.000 | 0.000 | 0.000 | 0.000 | 0.000 | 0.000 | 0.000 | 0.000 |
| 5 | 0.006 | 0.127 | 0.120 | 0.101 | 0.071 | 0.039 | 0.004 | 0.002 | 0.007 | 0.154 | 0.131 | 0.104 | 0.194 | 0.001 | 0.011 | 0.030 |
| 6 | 0.266 | 0.000 | 0.000 | 0.000 | 0.010 | 0.194 | 0.218 | 0.019 | 0.028 | 0.065 | 0.020 | 0.023 | 0.002 | 0.000 | 0.000 | 0.000 |
| 7 | 0.071 | 0.019 | 0.050 | 0.074 | 0.262 | 0.199 | 0.036 | 0.003 | 0.005 | 0.001 | 0.000 | 0.000 | 0.000 | 0.000 | 0.002 | 0.005 |
| 8 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 9 | 0.000 | 0.000 | 0.000 | 0.000 | 0.000 | 0.000 | 0.000 | 0.000 | 0.007 | 0.000 | 0.008 | 0.022 | 0.024 | 0.000 | 0.000 | 0.001 |
| 10 | 0.000 | 0.241 | 0.020 | 0.007 | 0.035 | 0.000 | 0.000 | 0.000 | 0.000 | 0.000 | 0.000 | 0.000 | 0.000 | 0.003 | 0.002 | 0.056 |

At the proper dose, the vancomycin, streptomycin, and polymyxin B treatments of host-free mixed culture were correlated with more “Ca. L. asiaticus” growth (FIGS. 22A-22B). Vancomycin and streptomycin interfere with cell wall synthesis and protein synthesis, respectively. Vancomycin is effective against a large number of Gram-positive bacteria, while streptomycin is effective against both Gram-negative and Gram-positive bacteria. There are two main mechanisms that might influence the growth of “Ca. L. asiaticus” when host-free mixed microbial cultures of it are treated with antibiotics. Some antibiotics can selectively eliminate faster-growing bacteria, which might reduce the overall competition with “Ca. L. asiaticus” growth, but it may be surmised that this mechanism is unlikely to be important given prior findings that link “Ca. L. asiaticus” growth with obligate interactions with other bacteria. Antibiotics might selectively enrich bacteria that interfere directly with “Ca. L. asiaticus” or that interfere with other bacteria on which “Ca. L. asiaticus” depends (e.g., FIG. 23A, upper left quadrant). Antibiotics might also selectively enrich “beneficial” bacteria that favor “Ca. L. asiaticus” growth (e.g., FIG. 23A, lower right quadrant)

When antibiotics are present at a low concentration, they can act as signaling molecules that in turn influence transcriptional regulation in bacteria, and this may play a role in the case of very low concentrations (0.02 and 0.05 μg/ml) of streptomycin. As signaling molecules, antibiotics can also alter interspecies interactions that ultimately influence the overall community composition.

It is notable that treatment with 0.02 μg/ml of streptomycin reduced the total diversity of the community by 70% and was associated with a nearly 6-fold increase in “Ca. L. asiaticus” growth. These findings suggest that the majority of the microbiota recovered from “Ca. L. asiaticus”—infected psyllids is not critical for “Ca. L. asiaticus” growth. Polymyxin B is active against some but not all Gram-negative bacteria; it is not active against “Ca. L. asiaticus” per se. It is likely that low concentrations of polymyxin B (0.5 μg/ml) and streptomycin (0.5, 1, and 2 μg/ml) benefit competing bacteria, including Bacillus sp. The growth of “Ca. L. asiaticus” was inversely correlated with the abundance of Bacillus cereus, which has been recovered previously from the psyllid host. Bacillus species including Bacillus subtilis and Bacillus cereus are able to produce antimicrobial compounds including hydrolytic enzymes (N-acyl homoserine lactonases). N-acyl homoserine lactonases can degrade N-acyl homoserine lactone (AHL), which causes the disruption of quorum-sensing signals, or quorum quenching. AHL is responsible for activation of the luxR gene, which encodes LuxR protein. LuxR and AHL are the two necessary components of the cell-to-cell communication system in bacteria. “Ca. L. asiaticus” contains the luxR gene, which encodes LuxR protein, but the/ux/gene, which encodes AHL, is absent from “Ca. L. asiaticus”. This implies that AHL from hosts or other microorganisms is responsible for activation of the luxR gene in “Ca. L. asiaticus” and cell-cell communication. Therefore, degradation of AHL by N-acyl homoserine lactonases and disruption of cell-cell communication of “Ca. L. asiaticus” are among the possible reasons for the inverse correlation between “Ca. L. asiaticus” and Bacillus cereus growth. Disruption of the quorum-sensing signals from competing bacteria (“quorum quenching”) results in significant changes in the microbial community, which in turn may contribute to the elimination of “Ca. L. asiaticus.” It has also been shown that exposure to a low concentration of antibiotics can upregulate the synthesis of quorum-quenching enzymes. Schneider et al. showed that under a growth condition with less than 0.65 μg/ml of polymyxin B or less than 2.5 μg/ml of streptomycin, the expression of a lactonase-homologous protein was induced and Bacillus sp. were enriched inside a mixed microbial culture, similar to the phenomena observed here. Thus, quorum quenching may be one of the mechanisms that affect “Ca. L. asiaticus” growth. However, Bacillus spp. including Bacillus cereus, have multiple antibacterial effects that may inhibit “Ca. L. asiaticus” growth; therefore—omics work (e.g., transcriptomics and metabolomics) will be performed to identify the actual mechanism behind the inverse correlation between “Ca. L. asiaticus” and Bacillus cereus growth within the mixed microbial cultures.

As described herein, adding streptomycin or polymyxin B to host-free mixed microbial cultures enriched Bacillus cereus and that this was responsible for the inhibited “Ca. L. asiaticus” growth. This is consistent with the cultures that were treated with 0.5, 1, or 2 μg/ml of streptomycin or 0.5 μg/ml of polymyxin B. It was not the case for the culture treated with 0.02 μg/ml of streptomycin, where a quorum-quenching mechanism may have been more important to “Ca. L. asiaticus” growth. The association between the Bacillaceae family and reduced “Ca. L. asiaticus” growth suggests that the enrichment of Bacillaceae such as Bacillus cereus within infected trees might serve as a biocontrol strategy for citrus greening disease.

In addition, a positive relationship between the presence of the Pseudomonas putida and “Ca. L. asiaticus” growth was observed. Samples treated with 1 or 2 μg/ml of streptomycin or 0.5 μg/ml of polymyxin B experienced a significant reduction of the Pseudomonadaceae family, consistent with a positive relationship between the Pseudomonadaceae family and “Ca. L. asiaticus” growth. Fujiwara et al. also observed that the elimination of Pseudomonadaceae from “Ca. L. asiaticus”-Ishi lmixed microbial culture decreased the survival rate of “Ca. L. asiaticus”. However, the mechanism of the benefit that “Ca. L. asiaticus” receives from Pseudomonadaceae is unknown. Regardless, this result suggests that co-culturing Pseudomonadaceae and “Ca. L. asiaticus” would promote “Ca. L. asiaticus” growth.

The present disclosure establishes a host-free mixed culture of “Ca. L. asiaticus” from infected psyllids, and established changes in the microbial community upon treatment with antibiotics. Based on diversity indices, it was concluded that antibiotic treatment changed the microbial structure and that host-free mixed microbial cultures treated with 0.5, 1, or 2 μg/ml of streptomycin or 0.5 μg/ml of polymyxin B had the lowest relatedness to other host-free mixed cultures. Moreover, concentrations of polymyxin B (<0.65 μg/ml) and streptomycin (<2.9 μg/ml) that were sub-inhibitory to Bacillus cereus enriched the Bacillaceae family (particularly Bacillus cereus) in “Ca. L. asiaticus” microbial communities and that these host-free mixed microbial cultures had lower “Ca. L. asiaticus” fold increases than other treatments or an untreated host-free mixed culture of “Ca. L. asiaticus.” This suggests that this bacterium is an inhibitor of “Ca. L. asiaticus” growth. Antibiotic treatment showed the structure of the cohabiting bacterial community plays an important role in “Ca. L. asiaticus” growth. Finally, these results, together with some from a previous publication, suggest that enrichment of the Bacillaceae family inside infected trees might serve as a paratransgenic approach to controlling citrus greening disease.

### Host Free Mixed Culture Preparation

The host-free mixed culture inoculum was prepared from live Asian citrus psyllids infected with “Ca. L. asiaticus” (strain psy62). These psyllids were maintained under greenhouse conditions. The infected psyllids were randomly selected from infected colonies. The selections were not sex-based and the ratio of the sex of the sampled insects is unknown. The presence of “Ca. L. asiaticus” in the inoculum was confirmed using qPCR as described in section 4.3. To prepare the inoculum, 20 infected psyllids were pooled together and crushed using a sterilized mortar and pestle (autoclaved at 121 for 15 min). The crushed psyllids were mixed with 20 ml of growth medium. The full growth medium recipe is provided in Table 5. The mixture was vortexed for 5 min to make sure that the inoculum was homogenized. This inoculum was used to inoculate 15-nil polystyrene tubes containing 5 nil of medium. The samples were incubated on a shaker (60 rpm) at room temperature (25° C.) for seven days. After seven days, these cultures were used to inoculate the second-cycle experiments. That is, the “first cycle” represents “Ca. L. asiaticus” growth when the infected ACP was used as the inoculum while the “second cycle” represents “Ca. L. asiaticus” growth when the first cycle was used as the inoculum. To take samples from the first cycle to inoculate the second cycle, the walls of the polystyrene tubes were physically scraped and the cultures homogenized to make sure that all possible biofilms on the wall were homogenized in the samples. All of these experiments were done as three independent replicates.

### Antibiotic Assay Experiments

To study the effects of antibiotics, the host-free mixed culture of “Ca. L. asiaticus” from the first cycle was used as the inoculum (20% inoculation). For these experiments the growth medium was supplemented with various concentrations of 4 different antibiotics: vancomycin (10, 25, 35, 50, 65, 75, or 100 μg/ml), streptomycin (0.02, 0.05, 0.5, 1, or 2 μg/ml), polymyxin B (0.5, 1, 2, or 4 μg/ml) or kasugamycin (0.02, 0.05, 0.5, 1, or 2 μg/ml). Similar to previous experiments, the samples were incubated on a shaker [60 rpm at room temperature (25° C.) for seven days]. The procedure that was used to take samples from the culture was similar to that described in Section 4.1. All of these experiments were done with three biological replicates.

DNA Extraction and qPCR

DNA was extracted from the samples using the manual method detailed here. Before DNA extraction, the samples were washed using a washing buffer (10 mM Tris, 100 mM NaCl and 1 mM EDTA, pH 8.0). Washed cells were transferred to lysing matrix E tubes (MP Biomedicals, USA) containing extraction buffer and beaten for 2 minutes. After being beaten, the samples were centrifuged at maximum speed (16,000 rcf) for 90 seconds, and the aqueous portion was transferred to new L5-ml microcentrifuge tubes. Then 10% sodium lauryl sulfate (SDS) (to make a final concentration of 2%) and 5 M NaCl (to a final concentration of 100 μM) were added to the samples, which were vortexed to mix them thoroughly. Proteinase K (Fisher Scientific, USA) was added to the samples to a final concentration of 0.2 μg/ml (1 μL of 20 μg/ml Proteinase K per 100 μL), and the samples were incubated at 56° C. for 1 hour with shaking. After incubation, one volume of phenol/chloroform/isoamyl alcohol (25:24:1, pH 8.0) was added to the samples. The samples were centrifuged at full speed (16,000 rcf) for 10 min, and the aqueous phase was collected carefully. One volume of chloroform/isoamyl alcohol (24:1) was used to remove the phenol residue. RNase (Fisher Scientific, USA) (10 μg) was added and incubated at 37° C. for 30 min to remove RNA in samples. Sodium acetate (0:1 volumes, 3 M, pH 5.5) and 2.5 volumes of ice-cold 100% ethanol were added to the samples. The samples were kept at −80° C. overnight. After that the samples were centrifuged at 4° C. for 10 min at maximum speed (16,000 ref) and washed twice with 0.15 mL of 70% ethanol (with a 5-min 4° C. spin (16,000 rcf) between washes). The samples were air-dried and resuspended in TE buffer. The extracted DNA was used for qPCR. Quantitative PCR using the following primer pair was performed to quantify “Ca. L. asiaticus” DNA in each sample: Las1F: 5′-GGT TTT TAC CTA GAT GTT GGG TAC T-3′ (SEQ ID NO: 43) and Las 1R5′-CTT CgC AAC CCA TTG TAA CC-3′ (SEQ ID NO: 44). Both of the primer sequences are conserved in “Ca. L. asiaticus” and were designed to amplify the Las-specific DNA fragment (140-bp) from its 16S rRNA gene. The reaction mixture components and target concentrations were a qPCR master mix (Power SYBR Green PCR Master Mix, Applied Biosystems, USA) with a final concentration of 1× and forward primer and reverse primers with a final concentration of 0.2 μM each. The DNA volume for each reaction mixture was 2 μl. The cycling parameters used to run the quantitative PCR were 95° C. for 3 min, followed by 40 cycles of 95° C. (denaturation) for 15 s and 57° C. and 68° C. for 20 s.

The specific 16S-rRNA amplicon (1160-bp) for “Ca. L. asiaticus” was obtained using PCR with high-fidelity DNA polymerase and the conventional primers (O11, O12c), purified and used as the molecular standard for qPCR. A 10-fold dilution series was prepared to create the standard curve. The number of GE per insect was calculated based on the standard curve, given that each “Ca. L. asiaticus” has three identical copies of the 16 S rRNA gene in its genome.

### Cloning and Sequencing

Clone libraries were constructed in order to determine the specificity of the DNA fragments amplified by O11/O12c (1160 bp) and Las1R/Las1F (140 bp). PCR and qPCR products extracted at 7 days from experiments using mixed microbial cultures from the samples not treated with antibiotic and the samples treated with 50 μg/ml of vancomycin were used as a gDNA template. These were gel-purified using the QiAquick Gel Extraction Kit (Qiagen, Germany) and cloned into the pCR™4-TOPO™ vector (Invitrogen™), followed by transformation into One Shot™ TOP10 competent Escherichia coli cells (Invitrogen™), using the manufacturer's protocols. Plasmids containing the insert were sent for sequencing (WSU Genomics Core, USA). Sequences were analyzed using MEGA 7.0 and compared to known sequences in GenBank using the Basic Local Alignment Search Tool algorithm against the nucleotide collection database (BLASTn) on the National Center for Biotechnology Information (NCBI) website. Sequences were deposited in NCBI's GeneBank database under submission number SUB_2259407.

Genomic DNA Extraction and 16S rRNA Sequencing

For gDNA extraction, 250 μl of each homogenized culture was extracted using a Qiagen MagAttract PowerMicrobiome kit (QUIAGEN, USA). After extraction, samples were quantified using the Quant-iT PicoGreen dsDNA Assay kit (Invitrogen, USA). The DNA extraction and libraries were prepared by the University of Michigan Host Microbiome Core as described previously. Briefly, the V4 region of the 16s rRNA gene was amplified from each sample using a dual indexing sequencing strategy. The PCR reactions were composed and performed as described in the previous protocol.

Amplicon samples were normalized using a SequalPrep Normalization Plate Kit (Life Technologies) following the manufacturer's protocol for sequential elution. The samples were pooled, and the concentration of the pooled samples was determined using the Kapa Biosystems Library Quantification kit for Illumina platforms (KapaBiosystems). The sizes of the amplicons in the library were determined using the Agilent Bioanalyzer High Sensitivity DNA analysis kit (Agilent). Libraries and sequencing reagents were prepared according to Illumina's protocols (“Preparing Libraries for Sequencing on the MiSeq” and “16S Sequencing with the Illumina MiSeq Personal Sequencer”) as described previously Amplicons were sequenced on the Illumina MiSeq platform using a MiSeq Reagent 222 kit V2 (catalog no. MS-102-2003) for 500 cycles according to the manufacturer's instructions with modifications for the primer set. FASTQ files were generated for paired-end reads.

### Analysis of Microbial Community

Raw sequence files (FASTQ files) were deposited in the Sequence Read Archive database under project SUB_6184324. Sequences were analyzed using mothur v.1.39. Briefly, filtered sequences were dereplicated and aligned to the most recent SILVA-based reference alignment (silva.nr_v132.align). The sequences were then screened to remove those that did not align to positions 11894-25319 of the reference alignment, filtered to remove non-informative columns, preclustered to >99.0% identity (allowing 2 differences), and dereplicated. Chimeras were identified and removed using UCHIME as implemented in mothur v.1.39 in self-referential mode. Filtered sequences were classified against the SILVA (v132) reference taxonomies using a naive Bayesian classifier implemented within mothur with an 80% bootstrap cutoff, and sequences that were not bacteria were removed using remove.lineage. OTUs were identified using a 97% similarity rate and used for downstream community analyses. OTUs were classified based upon the sequence classifications described above. Alpha diversity metrics (species observed, Chao and inverse Simpson) and beta diversity metrics (Bray-Curtis) were computed in mothur using subsampled sequences (n=44 898). Principal component analysis (PCA) imaging of the beta diversity metrics (Bray-Curtis) was performed based on Spearman's correlation matrix using XLStat 2015.

Table 6 as follows shows an example composition of a medium (1 L) for “Ca. L. asiaticus” growth.

TABLE 6

| INGREDIENT | Amount | |
|---|---|---|
| Alpha-ketoglutaric acid | 2 | g |
| ACE buffer | 10 | g |
| KOH | 3.75 | g |
| Salt mixture (100X) | 10 | ml |
| Phosphate buffer, IM (pH 7.0) | 10 | mL |
| DI water | Adjust to 880 | mL |
| Adjust the pH of above solution to 7.0 and autoclave at | | |
| 121° C. After cooling, aseptically add: | | |
| Fetal bovine serum | 25 | mL |
| Hink'sTNM-FH insect medium | 75 | mL |
| Vitamin mixture (100X) | 10 | mL |
| Trace mineral mixture (100X) | 10 | mL |

Table 7 as follows shows an example composition of salt, trace mineral and vitamin mixtures used in “Ca. L. asiaticus” growth medium.

TABLE 7

| | Concentration |
|---|---|
| 100x Salt mixture | (g/L) |
| KC1 | 38.0 |
| NH₄C1 | 20.0 |
| NaH₂PO₄ · H₂O | 6.9 |
| CaCl₂ · 2H₂O | 4.0 |
| MgSO₄ · 7H₂O | 20.0 |

| 100x Trace mineral mixture | g/L |
|---|---|
| Nitrilotriacetic acid (NTA) | 1.500 |
| - pH NTA solution to 10, then add: | |
| MnCl₂ · 4H₂O | 0.100 |
| FeSO₄ · 7H₂O | 0.300 |
| CoC1₂ · 6H₂O | 0.170 |
| ZnCl₂ | 0.100 |
| CuSO₄ · 5H₂O | 0.040 |
| AlK(SO₄)₂ · 12H₂O | 0.005 |
| H₃BO₃ | 0.005 |
| Na₂MoO₄ | 0.090 |
| NiCl₂ | 0.120 |
| NaWO₄ · 2H₂O | 0.020 |
| Na₂SeO₄ | 0.100 |

| - pH solution to 7.0 | |
|---|---|
| 100x Vitamin mix | mg/L |
| Biotin | 2.0 |
| Folic acid | 2.0 |
| Pyridoxine hydrochloride | 10.0 |
| Riboflavin | 5.0 |
| Thiamine hydrochloride | 5.0 |
| Nicotinic acid | 5.0 |
| DL-calcium pantothenate | 5.0 |
| Vitamin B₁₂ | 0.1 |
| p-Aminobenzoic acid | 5.0 |
| Thioctic (lipoic) acid | 5.0 |

### Example 5

### Controlled Replication of “Candidatus Liberibacter asiaticus” DNA in Citrus Leaf Discs

### Bacterial Strains and Culture Condition

Liberibacter crescens BT-1 (ATCC® BAA-2481™) was cultured in liquid BM7 medium at 28° C. and 20% O₂ tension. One Shot™ TOP 10 chemically competent Escherichia coli (Invitrogen, CA, USA) was cultured in Luria-Bertani (LB) liquid medium supplemented with 50 mg/ml ampicillin at 37° C. with agitation at 250 rpm.

### Establishment and Maintenance of Citrus Trees

Leaves from Citrus sinesis (L.) Osbeck (Hamlin) trees were maintained at the Citrus Research and Education Center, Lake Alfred, Fla., USA. Trees were kept in outside cages (semi-field conditions) to allow seasonal responses to temperature and light in a facility approved by the United States Department of Agriculture-Animal and Plant Health Inspection Service. The trees were inoculated by grafting with infected material and tissue harvesting initiated 9 months later when the trees started to show symptoms consistent with HLB. Trees were trimmed regularly (every three months) to stimulate new shoots. Plants were irrigated twice weekly and fertilized once every week using 20-10-20 NPK fertilizer (Peter's Fertilizer, Allentown, Pa., USA). Plant material was harvested in the morning and shipped overnight from Florida to Washington, refrigerated upon arrival and used for experimentation within seven days.

### Leaf Disc Assay

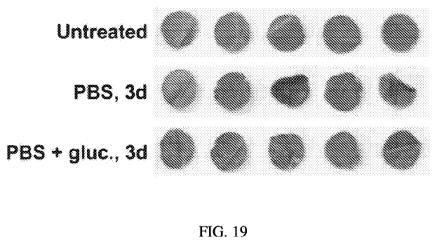

“Ca. L. asiaticus”-infected leaves were surface sterilized as follows: Leaves were soaked in 70% ethanol for 15 mM, rinsed with autoclaved water 2-3 times and transferred to a sterile container containing 10% bleach with 0.01% Tween-20 for 15 mM, then washed four times with sterile deionized water to remove the bleach from the leaf surface. Surface sterilized leaves were punched with sterile and disposable 5 mm leaf punches (Integra Miltex, PA, USA) and then carefully mixed in order to assure that the average bacterial load among groups of 5 leaf discs was equivalent. Subsequently, groups of 5 leaf discs were transferred into individual wells of 12-well plates containing 1.5 ml per well of different test media and incubated for 3 d. The basal PBS consisted of Na₂HPO₄ (8.1 mM), KH₂PO₄ (1.47 mM), KCl (2.7 mM), NaCl (136.8 mM), CaCl₂) (0.9 mM) and MgCl₂ (0.5 mM). All incubations were performed in the dark to prevent photosynthetic activity in the leaf discs from affecting “Ca. L. asiaticus” DNA replication. All incubations were done in regularly calibrated tri-gas incubators (Panasonic Health Care Corporation, IL, USA) adjusted to 28° C.; for microaerobic incubations, oxygen was displaced by nitrogen gas. Maintenance of natural leaf color over 3 days of incubation is consistent with maintenance of general tissue integrity and viability for at least 3 days (FIG. 19). The response of “Ca. L. asiaticus” to different conditions was measured by increases in gross “Ca. L. asiaticus” DNA content by qPCR.

### Extraction of Total DNA

To extract total DNA from citrus leaf discs, two to five (depending on type of experiment) “Ca. L. asiaticus”-infected leaf discs, stored at −20° C. before extraction, were placed into screw cap Lysing Matrix H tubes (MP Biomedicals, CA, USA), then homogenized using a Fastprep-24 System (MP Biomedicals, CA, USA) for 60 s at 6 m·s⁻¹. Leaf discs were homogenized dry. Following homogenization, either 600 or 200 μl extraction buffer was added to samples containing the equivalent of 5 or 2 leaf discs, respectively. Subsequent extraction of DNA was performed using the Wizard Genomic DNA Purification Kit (Promega Corp., WI, USA) according to the manufacturer's protocol. Genomic DNA of L. crescens and C. burnetii was extracted using the Quick-DNA™ Fungal/Bacterial Miniprep Kit (Zymo Research Corp., CA, USA). The concentration of sample DNA was determined using a NanoDrop spectrophotometer (Thermo Fisher Scientific, MA, USA).

### Quantitative and Conventional PCR

Primers used in this study are identified as SEQ ID NOS: 31-42:

| | |
|---|---|
| SEQ. ID NO: 31: | GCGCGTATGC AATACGAGCG GCA |
| SEQ. ID NO: 32: | GCCTCGCGAC TTCGCAACCC AT |
| SEQ. ID NO: 33: | CTTATCACCG GCAGTCCCTA TAAAG |
| SEQ. ID NO: 34: | CAGCTCGTGT CGTGAGATGT TG |
| SEQ. ID NO: 35: | TTGTTTCGAT ATCCGAATCT ATGCGGG |
| SEQ. ID NO: 36: | ATATTTGATT CTCGTGCAAT TTGTAGCAAC |
| SEQ. ID NO: 37: | TCTAAAGCAG GACGTTTGTG T |
| SEQ. ID NO: 38: | TGAGTATATT TGATTCTCGT GCAA |
| SEQ. ID NO: 39: | ACTTGCCAAA CCAACCGTTG AAG |
| SEQ. ID NO: 40: | ACCAATTCTT GTGATATTCG GGC |
| SEQ. ID NO: 41: | CGCTCTCGAT GGGATTGGAA |
| SEQ. ID NO: 42: | CTGAGGTTTC TGTCCCCGTC |

All Quantitative-PCR (qPCR) reactions were performed using a CFX318 real-time PCR Detection System (Bio-Rad, CA, USA). Briefly, 10 μl qPCR reactions contained 5 μl 2× SYBR green qPCR master mix (IQ™ SYBER® GREEN supermix, Bio-Rad, CA, USA), 0.2 μM of each primer (qPCR-CD16-00155 F and qPCR-CD16-00155 R) and 0.2 μg template DNA. The amplification conditions for 16S rDNA followed published protocols (e.g., Orce et al., 2015 and Jagoueix et al.). All reactions were performed in triplicate with a positive, autoclaved infected leaf discs as a negative, and “no template” controls.

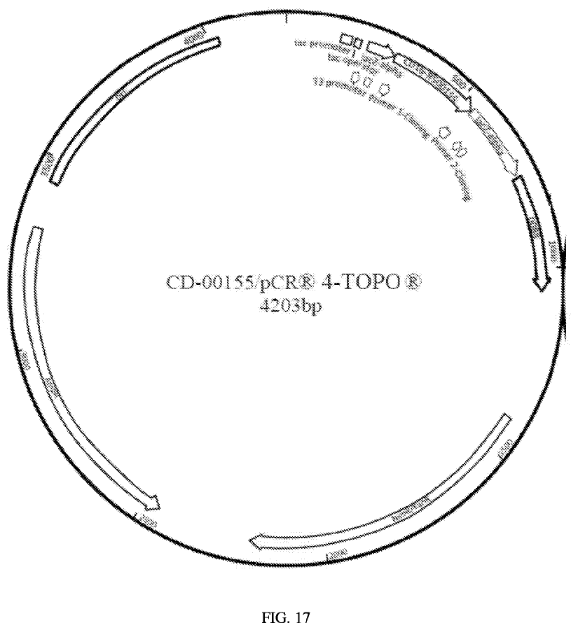

Absolute quantification of “Ca. L. asiaticus” was based on qPCR of the hypothetical gene CD16-00155 (“Ca. L. asiaticus”, strain A4). The CD16-00155 sequence was amplified from total DNA extracted from the midrib of “Ca. L. asiaticus”-infected leaves by conventional PCR using 0.2 μg of DNA template, 0.2 μM primer, 0.25 mM dNTP, 1× buffer, and 0.125 μl of Phusion high-fidelity DNA polymerase (Thermo Fisher Scientific, MA, USA). The amplification product was cloned into the pCR™ 4-TOPO® vector (Invitrogen, CA, USA) and then transformed into E. coli TOP10 cells (Invitrogen, CA, USA) (FIG. 17). Transformants were selected using LB medium supplemented with 50 μg. ml⁻¹ ampicillin at 37° C. and extracted plasmid sequenced to validate the insertion. The plasmid was extracted from E. coli using the PureLink kit (Invitrogen, CA, USA) according to the manufacturer's instructions. Plasmid concentration was determined using a NanoDrop spectrophotometer (Thermo Fisher Scientific, MA, USA), and plasmid copy numbers were calculated based on the molecular weight of the plasmid. A standard curve was generated by serial dilution of the plasmid and the absolute quantification of “Ca. L. asiaticus” extrapolated from the standard curve and presented as genome equivalents (GE).

### Primary Metabolite Derivatization and Gas Chromatography Time-of-Flight Mass Spectrometry Analysis

Extraction was carried out using a slight modification of an established procedure (e.g., Lee and Fiehn, 2008). To assure metabolite profiles represented that of infected leaf tissue, leaves subjected to metabolite profiling were pre-screened for the presence of “Ca. L. asiaticus” DNA. After incubation, a defined amount of powdered freeze-dried citrus leaves (ca. 5-14 mg) was suspended in 500 μL of extraction solvent (methanol:2-propanol: water, 5:2:2 v:v:v). After adding 1.0 μg of the internal standard salicylic acid-d6 (C/D/N Isotopes, Canada), the material was extracted by shaking at room temperature for 10 mM (Vortex) and sonication at room temperature for 10 mM (Branson 5510 sonication bath, Branson Ultrasonics Corp, CT, USA). The extracts were then centrifuged for 10 min at 21,000×g, and the supernatants transferred into new vials. The extracts were dried under vacuum. Dry residues were suspended in 500 μL of 50% aqueous acetonitrile and re-extracted as above by sequential vortexing and sonication. The debris was again removed by centrifugation, and the supernatants dried under vacuum. The dry residues were suspended in 10 μL, O-methoxylamine hydrochloride (30 mg. mL⁻¹ in pyridine, Sigma, MO, USA) and incubated for 90 min at 30° C. and 1000 rpm. Subsequently, samples were derivatized with 90 μL of N-methyl-N-(trimethylsilyl) trifluoroacetamide (MSTFA) with 1% trimethylchlorsilane (TMCS) (Thermo Fisher Scientific, MA, USA) for 30 mM at 37° C. and 1000 rpm. Samples were spiked with a mixture of linear alkanes for the calculation of retention indices. Gas chromatography-mass spectroscopy analysis was performed using a Pegasus 4D time-of-flight mass spectrometer (LECO, MI, USA) equipped with a Gerstel MPS2 autosampler and an Agilent 7890A GC oven. The derivatization products were separated on a 30 m, 0.25 mm i.d., 0.25 μm df Rxi-5Sil® column (Restek, CA, USA) with an IntegraGuard® pre-column using ultrapure He at a constant flow of 0.9 mL min⁻¹ as carrier gas. The linear thermal gradient started with a one-minute hold at 70° C., followed by a ramp to 300° C. at 10° C.·min⁻¹. The final temperature was held for 5 min prior to returning to initial conditions. Mass spectra were collected at 70 eV and 17 spectra. s⁻¹. The injection port was held at 240° C., and 2 μL of the sample were injected at an appropriate split ratio. After deconvolution and peak alignment, a typical experiment yielded over 800 distinct chemical features/analytes. Peak alignment and spectrum comparisons were carried out using the Statistical Compare feature of the ChromaTOF® software (LECO, MI, USA). Peak identification was conducted using the Fiehn primary metabolite library with an identity score cutoff of 600. Based on comparison to reference mass spectra, over 200 analytes could be assigned a probable identity, with confirmation of specific compounds accomplished by comparison to an in-house custom library of ˜120 authentic standards. Approximately 40 primary metabolites were consistently and reliably detected in all samples and thus included in the final analysis. The internal standard and the initial tissue weight were used for normalization. Statistical analyses with selected metabolites were carried out using MetaboAnalyst 4.0.

Primary Metabolite Profiles Derived from Healthy or “Ca. L. asiaticus”-Infected Leaves in Response to Glucose

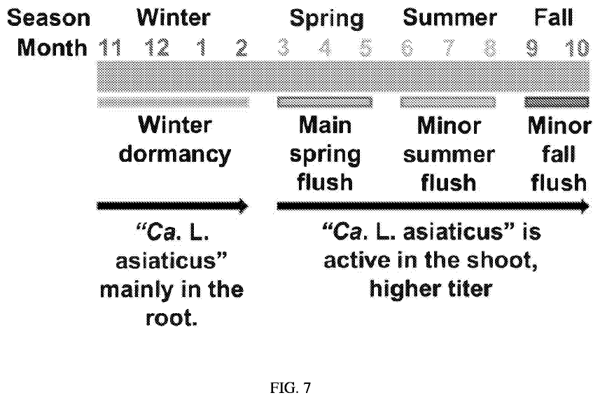

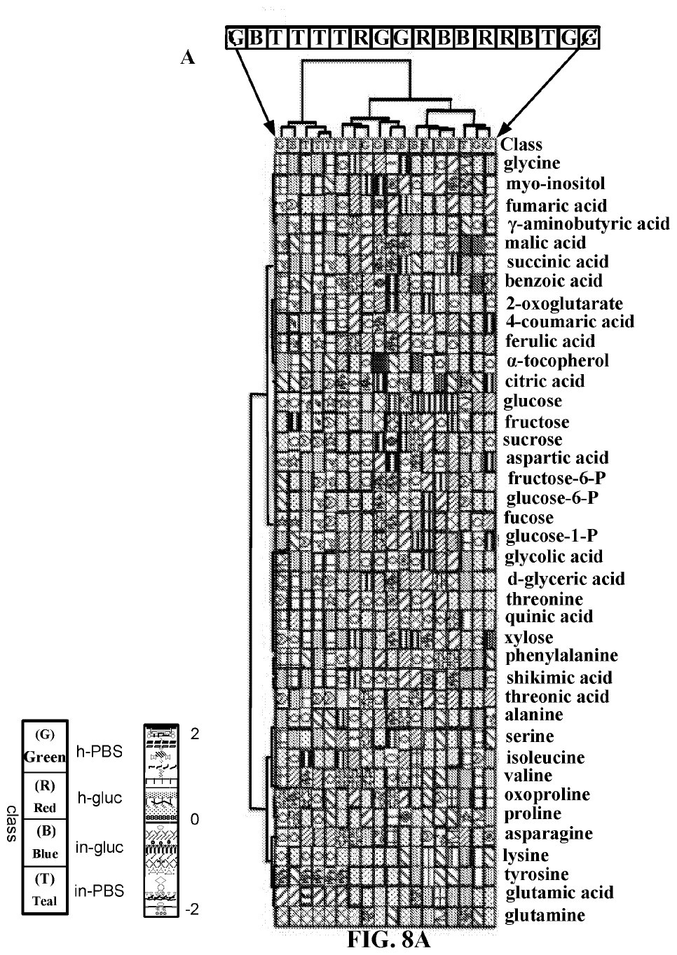

This study is based on citrus trees maintained under semi-field conditions with exposure to natural light. FIG. 7 summarizes seasonal characteristics in citrus tree development and physiology and reported changes in “Ca. L. asiaticus” loads in trees. The main leaf flush occurs in the early spring and minor flushes follow in the summer and early fall. Flushing is generally synchronized with higher populations of “Ca. L. asiaticus” in the leaves. Upon measuring pathogen titer in March and June of 2018 (data not shown), seasonal variability in “Ca. L. asiaticus” titer with moderately higher bacterial loads in the spring compared to the summer was observed and in agreement with previous results from Florida.

Several studies have identified alterations in primary metabolite content in “Ca. L. asiaticus”-infected plant tissues. Because defense responses in plants can be energy dependent, changes in plant central carbon metabolism can affect initiation of defense responses after encounter with a pathogen. Moreover, pathogens could take advantage of the resulting rearrangements in plant physiology upon infection in order to improve survival. Therefore, an understanding of whether “Ca. L. asiaticus” infection affects host responses to specific nutrients can reveal pathogen-induced manipulation of host metabolic status and capacity.

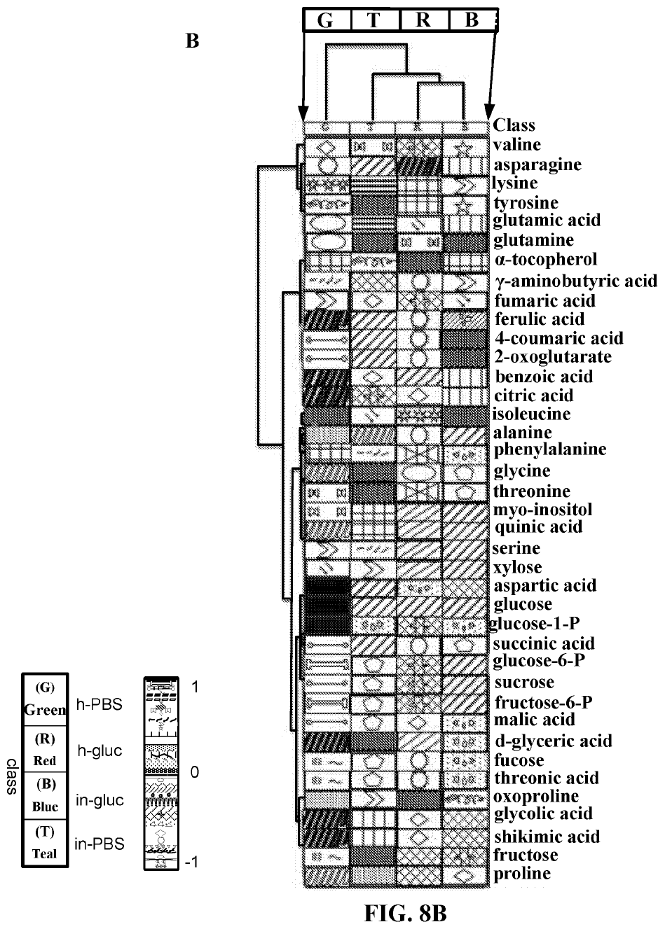

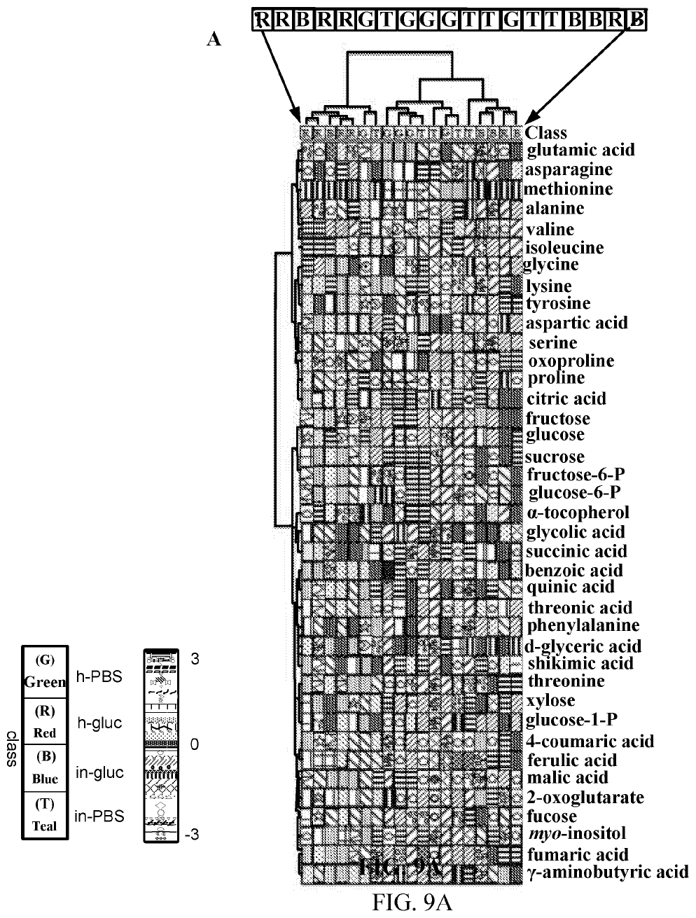

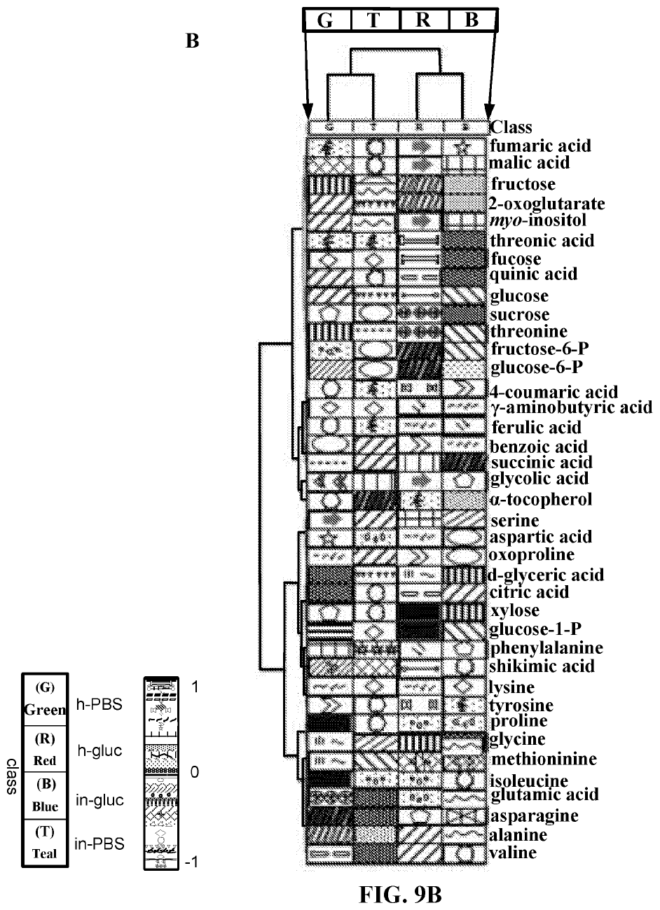

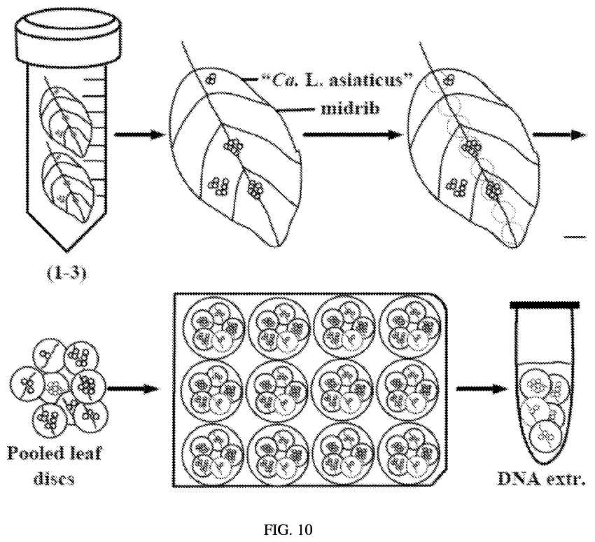

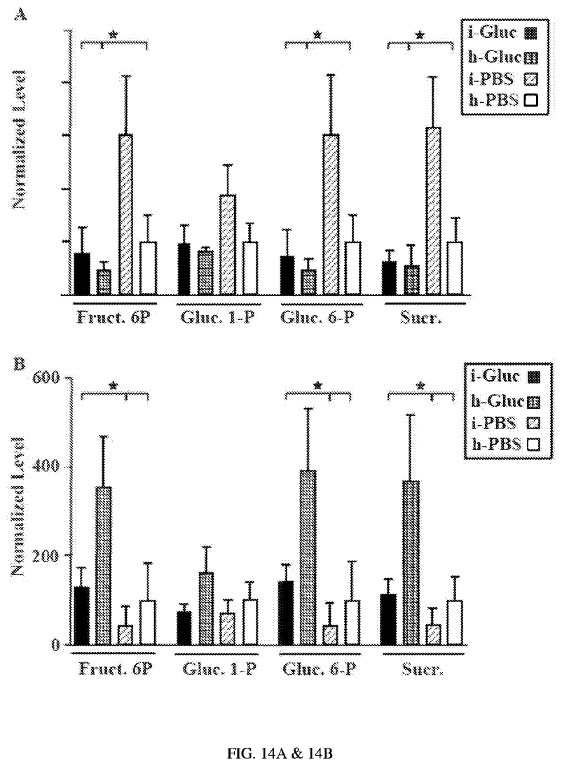

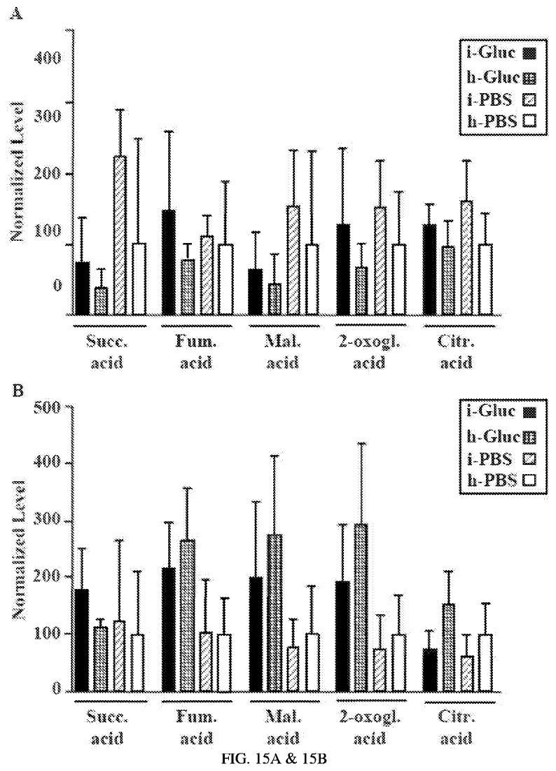

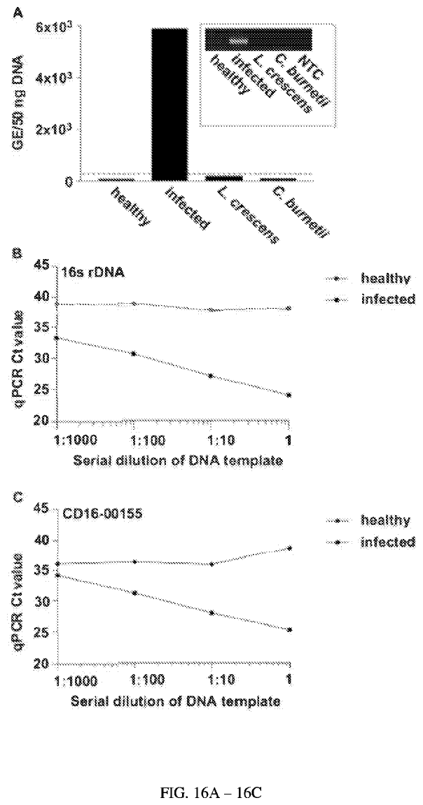

Glucose is a critical carbon source in most organisms, including citrus. To test the effect of “Ca. L. asiaticus” infection on the ability of citrus leaf tissue to respond to glucose, gas chromatography-mass spectrometry (GC-MS) was used to assess primary metabolite profiles of healthy or “Ca. L. asiaticus”-infected leaves in the presence or absence of glucose (FIGS. 8A-8B and 9A-9B). The analysis was performed with samples harvested during months correlating with critical stages in “Ca. L. asiaticus” and citrus biology (e.g., FIG. 7). While early spring correlates with tree emergence from winter dormancy, migration of “Ca. L. asiaticus” from the roots to the canopy and apparent growth in the flush, the summer represents onset of higher temperatures and extended daylight (geographical region FL, USA) with direct effects on tree metabolism and photosynthetic activity. Leaf discs were cut from the midribs of surface decontaminated leaves that had also been pre-screened for colonization by “Ca. L. asiaticus”, thus avoiding performing metabolite analysis on tissue that is not directly colonized by the pathogen. Leaf discs were then incubated for 3 d in either plain PBS or PBS supplemented with 10 mM glucose. Incubations were performed in the dark to prevent photosynthetic activity, a variable that could mask responses and a process not consistent with the end goal of this study (i.e., screening of variables that facilitate “Ca. L. asiaticus” DNA replication). FIGS. 8A-8B and 9A-9B show heatmaps for corresponding metabolite profiles measured in March and June 2018, respectively. Consistent clustering between samples replicates was not observed in March (FIG. 8A), suggesting “Ca. L. asiaticus” does not significantly affect primary metabolite profiles or the ability of young/flowering leaf tissue to metabolize glucose. When signal intensities for individual metabolites from all replicates in each treatment group were averaged (FIG. 8B), clear separation between treatment groups were observed but the overall change in metabolite abundances was minimal. In June, “Ca. L. asiaticus” infection resulted in a shift in metabolite profile consistent with differential ability of infected tissue to respond to glucose (FIG. 9A). Compared to healthy tissue, “Ca. L. asiaticus”-infected material generally showed reduced levels of glucose and immediate metabolites of glucose (glucose 1-phosphate, glucose 6-phosphate, and fructose 6-phosphate). Additionally, several amino acids (including lysine, tyrosine, isoleucine, glycine, valine, alanine, methionine, proline, aspartic acid, and serine) showed a trend toward reduced levels in infected tissue. Healthy tissue incubated with glucose displayed an overall different metabolite profile with elevated levels of most detected metabolites. Pathogen-infected tissue incubated without glucose generally grouped with healthy tissue incubated without glucose. As for the month of March, averaging signal intensities for individual metabolites from all replicates in each treatment group (FIG. 9B) revealed moderate differences in the abundances of specific metabolites. However, averaging also highlighted the effect of “Ca. L. asiaticus” infection in reducing pools of critical metabolites (including citric acid, glucose-1-P, and the amino acids serine, phenylalanine, lysine, tyrosine, proline and glycine) in both the absence and presence of glucose. FIGS. 14A-14B and 15A-15B show quantitative comparisons of select saccharides (FIGS. 14A-14B) or TCA cycle (FIGS. 15A-15B) intermediates in leaf discs incubated in the absence or presence of glucose. In March, but not in June, infected leaf discs showed elevated levels of glycolytic intermediates under control conditions (absence of glucose). During incubation with glucose, tissue analyzed in March showed reduced levels of glycolytic intermediates while tissue analyzed in June showed elevated levels of Fructose-6-P, Glucose-6-P and sucrose in non-infected tissue incubated with glucose. TCA cycle intermediates exhibited extensive variability between treatment groups. Quantitative differences in relative abundance of selected sugars were variable and within 2-3 fold, thus considered moderate in nature. Overall, the observed shifts in metabolite profile are consistent with “Ca. L. asiaticus” infection affecting leaf metabolism and responses to glucose.

Design of a Leaf Disc Assay to Test the Effect of Physicochemical and Nutritional Condition on “Ca. L. asiaticus” DNA Replication

A leaf disc-based assay, exploiting a natural niche for “Ca. L. asiaticus”, was developed to identify physicochemical and nutritional requirements for “Ca. L. asiaticus” replication (FIG. 10). Although leaf tissue naturally contains all the nutrients required by “Ca. L. asiaticus” for replication, supplementation of limiting nutrients is expected to stimulate replication thus allowing detection of pathogen responses without detailed knowledge of “Ca. L. asiaticus” metabolic requirements and capabilities.

Measurement of genome equivalents (GE) is a widely used method to identify gross increases in DNA replication (although not necessarily cell division). An oligonucleotide primer pair was designed specific to the conserved “Ca. L. asiaticus” hypothetical gene CD16-00155 (strain A4) as a basis for quantification of “Ca. L. asiaticus” DNA. Nucleotide sequence BLAST with CD16-00155 did not result in detection of similar sequences in other bacteria (including “Ca. L. americanus”, “Ca. L. solanacearum”, and L. crescens), consistent with the utility of using detection of CD16-00155 for highly specific detection and quantification of “Ca. L. asiaticus”. To validate the specificity and selectivity of the primers designed for detection of CD16-00155, PCR amplification was performed on 50 ng of total DNA (tDNA) isolated from healthy or “Ca. L. asiaticus”-infected leaves, tDNA isolated from L. crescens and the unrelated (animal) pathogen Coxiella burnetii (FIG. 16A). Amplification of CD16-00155 allowed detection of “Ca. L. asiaticus” DNA with expected specificity and selectivity in that amplification was only observed in “Ca. L. asiaticus”-infected plant tissue. To assess reaction sensitivity, amplification was tested over a concentration gradient of template tDNA. Relative quantification of “Ca. L. asiaticus” based on CD16-00155 was as sensitive as that observed when using 16S rDNA as target (FIG. 16B-16C). Sequencing of the PCR and qPCR reaction products from amplification of CD16-00155 confirmed amplification of the target sequence (data not shown). To facilitate absolute quantification of “Ca. L. asiaticus” DNA, CD16-00155 was cloned into a plasmid vector from which a standard was generated (FIG. 17).

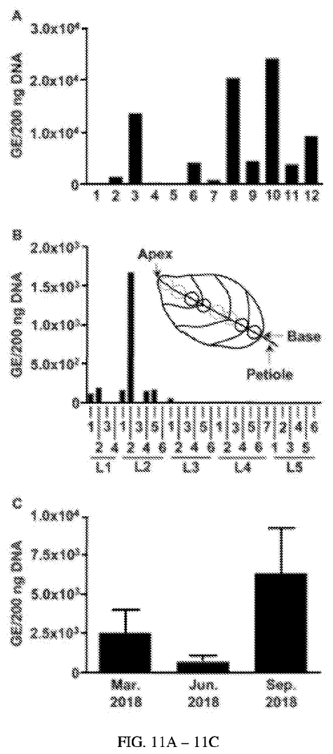

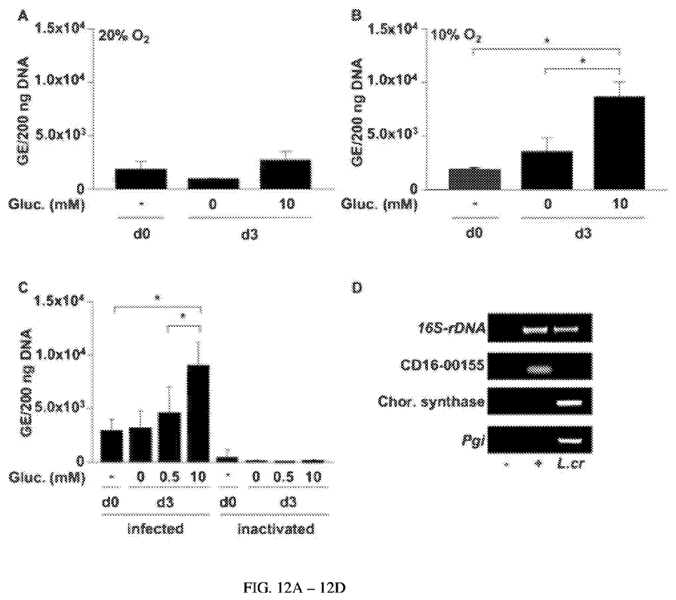

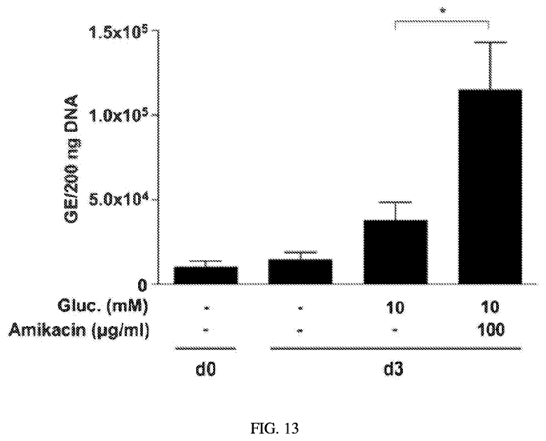

“Ca. L. asiaticus” infection of citrus is characterized by extensive variability in pathogen titer in shoots throughout the canopy. To establish the leaf disc assay platform with the lowest possible variability between replicate samples and independent experiments, the “Ca. L. asiaticus” load in leaves was characterized. Initially, the absolute bacterial load was quantified in different parts of the leaf including the midrib, leaf blade, petiole and stem based on quantitative detection of CD16-00155. In agreement with other reports, “Ca. L. asiaticus” was most and consistently abundant in the midrib (data not shown). To determine the variability of pathogen colonization among leaves, multiple leaves from different branches were randomly collected and surface sterilized, then two leaf discs were punched from the midrib of each leaf and tDNA extracted for GE analysis (FIG. 11A). Out of twelve tested leaves, four had “Ca. L. asiaticus” loads equal to or higher than 1×10⁴ GE/200 ng DNA, while the rest of the leaves had “Ca. L. asiaticus” load lower than 1×10² GE/200 ng DNA. In one leaf, the “Ca. L. asiaticus” load was below the detection limit. Additionally, upon quantifying the absolute “Ca. L. asiaticus” load along the midrib in segments equal to two leaf discs (5 mm diameter), it was observed that while some sections show a “Ca. L. asiaticus” load higher than 1×10³ GE/200 ng DNA, other parts show very low or even undetectable bacterial loads (FIG. 11B). To distribute the pathogen evenly between culture samples, leaf discs prepared from several leaves were pooled, then divided evenly between cultures to assure detectable and similar levels of “Ca. L. asiaticus” for any given experiment regardless of time of tissue harvest (FIG. 11C), greatly reducing overall assay variability (FIG. 10).

Stimulation of “Ca. L. asiaticus” DNA Replication within Leaf Discs

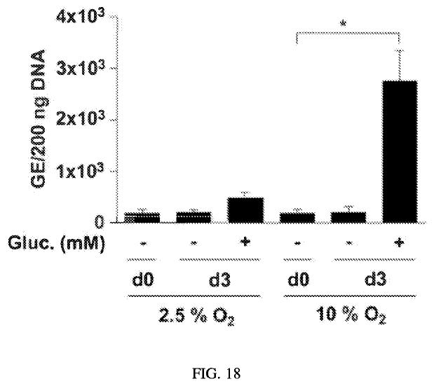

As exemplified by host cell-free replication of the bacterial obligate intracellular parasite C. burnetii, bacteria can exhibit highly specific physicochemical requirements for replication. “Ca. L. asiaticus” is adapted to citrus phloem sap, a microaerobic environment. Therefore, the dependency of “Ca. L. asiaticus” DNA replication on specific O₂ availability was tested (FIGS. 12A-12D). While no significant changes in GE were observed after incubation under normoxic (air/˜20% O₂) conditions, a 4.6-fold increase in bacterial load was observed between day 0 (d0, 1.9×10³±1.9×10² GE) and day 3 (d3, 8.7×10³±1.4×10³ GE) when leaf discs were incubated in the presence of glucose and the level of O₂ was reduced to 10% (FIG. 12A-12B), suggesting that “Ca. L. asiaticus” is indeed a microaerophile. Reducing available 02 to 2.5% did not positively affect replication (FIG. 18). “Ca. L. asiaticus” appears optimally adapted to an environment where the oxygen level is approximately 10%.

“Ca. L. asiaticus” may be able to utilize glucose directly or benefit from a product of glucose metabolism (e.g., ATP) following oxidation by the leaf tissue. Incubation of leaf discs from “Ca. L. asiaticus”-infected plants in PBS containing different concentrations of glucose revealed dose-dependent increases in GE (FIG. 12C). Relative to d0 (3×10³±5.8×10² GE), leaf discs incubated for three days with 10 mM glucose (9.5×10³±1.4×10³ GE) had significantly higher GE counts, equivalent to a 3.2-fold increases in DNA. Relative to d0, incubation with 0.5 mM glucose (d3, 4.7×10³±1.3×10³ GE) showed a non-significant 1.6-fold increase in GE counts, consistent with a dose-dependent effect of glucose on “Ca. L. asiaticus” DNA replication. Incubation of autoclaved leaf discs did not produce any increases in GE over the 3-day incubation. “Ca. L. asiaticus” may utilize glucose directly by importing glucose into the cytoplasm via a glucose/galactose transporter (e.g., CD16-03615, A4 strain). However, the possibility that leaf tissue actively converts glucose into a downstream metabolite used by “Ca. L. asiaticus” cannot be excluded.

Genome sequence analysis has revealed that “Ca. L. asiaticus” encodes a nearly complete glycolytic pathway, but is missing glucose-6-phosphate isomerase (pgi, EC 5.3.1.9). Based on mutational analysis in Escherichia coli, loss of pgi can have significant negative implications for utilization of glucose with corresponding re-arrangements of metabolic flux. Because the genome(s) of “Ca. L. asiaticus” has been obtained via metagenomics sequencing and pathogen isolates may differ in genetic makeup, PCR of several genes was used to validate expected presence or absence of genes, including pgi, between tDNA isolated from healthy or “Ca. L. asiaticus”-infected leaf tissue, and gDNA isolated from L. crescens (FIG. 12D). While pgi (using primers internal to the L. crescens ortholog), 16S-rDNA, and the gene encoding chorismate synthase were detected in L. crescens, only 16S-rDNA and the hypothetical sequence CD16-00155 were detected in tissue containing “Ca. L. asiaticus”. Among Liberibacter species, only L. crescens appears to encode chorismate synthase. Although based on lack of detection, given the positive controls (16S-rDNA and hypothetical gene CD16-00155) included in this experiment and the pattern of positive amplification between species and sample types, results strongly support absence of pgi from the “Ca. L. asiaticus” strain used in this study.

“Ca. L. asiaticus” does not appear to be sensitive to the antibiotic amikacin. In addition, antibiotics have been used to suppress the growth of specific bacteria and thus reduce the complexity of microbial communities in citrus. Therefore, it was tested if the response of “Ca. L. asiaticus” DNA replication was positively affected by the presence of amikacin during incubation (FIG. 13); leaves were pre-screened for the presence of pathogen DNA to further reduce assay variability. Incubation of leaf discs from “Ca. L. asiaticus”-infected plants in PBS containing 10 mM glucose in the absence or presence of amikacin showed that incubation with amikacin resulted in a 3.03-fold potentiation in “Ca. L. asiaticus” DNA content (d3 glucose, 3.8×10⁴±1.07×10⁴ GE; glucose and amikacin, 1.15×10⁵±2.8×10⁴ GE) (FIG. 13) after three days of incubation. Compared to the starting material, an overall 11.1-fold increase in GE was observed (d0, 1.03×10⁴±3.3×10³ GE; d3 glucose and amikacin, 1.15×10⁵±2.8×10⁴ GE). Amikacin might have a negative effect on bacteria that normally compete with “Ca. L. asiaticus” for nutrients and thus reduce the potential for DNA replication.

An assay based on incubating citrus leaf discs in solution was established to enable screening of parameters that affect replication of “Ca. L. asiaticus” DNA in situ. An increase in “Ca. L. asiaticus” DNA within leaf discs was observed under reduced oxygen availability (10% O₂), but not under normoxic (air) conditions. Moreover, glucose stimulated “Ca. L. asiaticus” replication in a dose-dependent manner in situ. Incubation with the antibiotic amikacin further stimulated “Ca. L. asiaticus” DNA replication, suggesting improved “Ca. L. asiaticus” activity when other bacteria are suppressed. A comparison between the metabolite profiles derived from healthy versus “Ca. L. asiaticus”-infected leaf discs following incubation with glucose revealed a trend consistent with a moderate alteration of metabolism in infected tissue. Collectively, these findings are consistent with a model in which “Ca. L. asiaticus” replicates optimally under microaerobic conditions and produces moderate changes in the metabolite makeup of its replicative environment, possibly as a means to increase availability of nutrients that promote pathogen replication and viability.

Several metabolomics studies have revealed changes in the metabolite profiles of citrus leaves and fruit juice after infection with “Ca. L. asiaticus” (Slisz et al., 2012) (Hijaz et al., 2013; Killiny, 2017). For example, concentrations of sugars, organic acids, amino acids and lipids can be altered in response to infection. Additional studies have demonstrated altered carbohydrate (e.g., glucose and fructose) content in “Ca. L. asiaticus” infected leaves (Fan et al., 2010; Albrecht et al., 2016). Levels of glucose and fructose have been shown to vary depending on the area of the infected leaves, and time after infection (Albrecht et al., 2016) suggesting that seasonal changes may additionally be affected by spatiotemporal activity within individual leaves. Results show that the levels of primary metabolites in “Ca. L. asiaticus”-infected leaves from trees exhibit some yet inconsistent variability when exposed to glucose (FIGS. 8A-8B and 9A-9B). Overall, these data show that “Ca. L. asiaticus” infection can affect the metabolite profile of infected tissue and the metabolic response of leaf tissue to specific nutrients, in this case illustrated by the response to incubation with glucose. As revealed upon averaging signal intensities from metabolites detected in March or June, the abundances of several metabolites (including quinic acid, myo-inositol, sucrose, glucose- and fructose-6-phosphate) shifted in opposite directions in the two months shown, indicating that responses to glucose treatment may be season-dependent.

“Ca. L. asiaticus” has been described to have limited capacity for aerobic respiration (Duan et al., 2009). Despite the lack of cytochrome bd (cydAB), a terminal oxidase typically associated with microaerobic metabolism, “Ca. L. asiaticus” was able to undergo DNA replication, but only under microaerobic condition (FIGS. 12A-12D). “Ca. L. asiaticus” DNA replication under microaerobic conditions despite absence of a terminal oxidase indicative of a microaerophilic lifestyle could be related to organism sensitivity to oxidative stress. Importantly, the cytochrome 0 ubiquinol oxidase encoded by “Ca. L. asiaticus” could also function under microaerobic conditions (Tseng et al., 1996).

“Ca. L. asiaticus” DNA replication was observed upon incubation of leaf discs with glucose. “Ca. L. asiaticus” is either able to metabolize glucose, predicted from metabolic pathway reconstruction (Fagen, Leonard, McCullough, et al., 2014), and analysis of E. coli mutants with defects in pgi (Charusanti et al., 2010; Long et al., 2018), or responds to a glucose-dependent alteration in leaf physiology, such as synthesis of ATP. Ability of “Ca. L. asiaticus” to utilize glucose is in agreement with gene expression profiling of “Ca. L. asiaticus” (Yan et al., 2013) demonstrating that genes encoding enzymes involved in glycolysis are expressed in planta. Similar to E. coli (Charusanti et al., 2010) and as predicted for “Ca. L. asiaticus” (Fagen, Leonard, McCullough, et al., 2014), the pathogen may adapt to loss of pgi by rerouting metabolic flux through the pentose phosphate pathway (PPP). In short, “Ca. L. asiaticus” may bypass the early conversions in glycolysis to generate glyceraldehyde-3-phosphate via the PPP (Fagen, Leonard, McCullough, et al., 2014), allowing “Ca. L. asiaticus” to produce pyruvate from glucose. Apparent absence of the PPP enzyme transaldolase (E. C. 2.2.1.2) in “Ca. L. asiaticus” (Fagen, Leonard, McCullough, et al., 2014) may compromise generation of glyceraldehyde-3-phosphate via PPP activity. Moreover, the absence in “Ca. L. asiaticus” of genes shown to have significance for detoxification of methylglyoxal (MG) (Jain et al., 2017) may predispose “Ca. L. asiaticus” to MG sensitivity and thus make metabolism of glucose a sub-optimal carbon source for this pathogen. Regardless, conservation of a nearly complete glycolytic pathway, including the enzyme that facilitated entry of glucose into the pathway, is consistent with oxidation of glucose by “Ca. L. asiaticus”. Genome sequence analysis based on metagenomics assembly showed that “Ca. L. asiaticus” is similar to “Ca. L. solanacearum” (Lin et al., 2011) in that it does not encode a phosphotransferase system (PTS), a common bacterial machinery for transporting carbohydrates (Kotrba et al., 2001). However, “Ca. L. asiaticus” does encode a single glucose/galactose transporter (CD16-03615, strain A4) (Zheng et al., 2014), suggesting that “Ca. L. asiaticus” can take up glucose. Recent analysis of broth-based culture of the Ishi-1 strain of “Ca. L. asiaticus” (Fujiwara et al., 2018) lends support to the finding that carbohydrates, including glucose, are important for optimal growth of “Ca. L. asiaticus”. Because “Ca. L. asiaticus” Ishi-1 does not harbor a pro-phage that appears to have a major impact on pathogen culturability (Fleites et al., 2014; Fujiwara et al., 2018), the hypothesis that glucose can be used directly by the “Ca. L. asiaticus” strain used in this study cannot be tested until a chemically defined medium that supports axenic growth of a wider variety of “Ca. L. asiaticus” strains becomes available.

The “Ca. L. asiaticus” genome encodes an apparently intact ATP/ADP transporter (nttA) (Duan et al. 2009; Vahling et al. 2010; Jain et al. 2017), suggesting the pathogen acts like an “energy parasite” by importing ATP directly from the host akin to the obligate intracellular bacteria Rickettsia prowazekii (Plano and Winkler, 1991; Driscoll et al., 2017) and Chlamydia trachomatis (E. R. I. Lee and McClarty, 1999). It is possible that leaf tissue converts glucose to ATP, and therefore that the increase in “Ca. L. asiaticus” GE within leaf discs as observed in this study is an indirect response to glucose.

Documented seasonal variability in “Ca. L. asiaticus” loads in infected trees (Lopez-Buenfil et al., 2017) may affect the utility of leaf discs prepared from citrus in screening physicochemical and nutritional conditions that affect in situ replication. Indeed, it was observed optimal assay responses between March and September. This limitation could be based on the natural biology of the interaction between “Ca. L. asiaticus” and citrus trees, including increased pathogen activity in the spring and early summer when the flush develops and trees are at their highest level of activity. Use of greenhouse- or growth-chamber cultivated plants that are subject to less seasonal variability may allow assay responses that are consistent throughout the year.

In conclusion, a strategy was developed to assess “Ca. L. asiaticus” responses to physicochemical and nutritional variables in the context of leaf tissue. Because responses in “Ca. L. asiaticus” DNA replication were observed within three days, the methods presented herein are suitable for medium-throughput screening of conditions that influence pathogen DNA replication in situ. “Ca. L. asiaticus” responses to different conditions were determined by measuring bacterial GE by qPCR targeting a single-copy hypothetical gene that appears unique to “Ca. L. asiaticus”, thus reducing the likelihood of detecting DNA related to organisms other than “Ca. L. asiaticus”. Unlike published methods (e.g., (Zhang et al., 2014)) to assess “Ca. L. asiaticis” responses to chemical stimuli (e.g., antibiotics) that yield a qualitative output (e.g., disease transmission), the assay described herein allows such analysis under conditions where pathogen replication is activated and the level of activation is quantitated at the “Ca. L. asiaticus” cellular level. Methods other than qPCR would have to be used to correlate DNA replication with potential cell division. Because “Ca. L. asiaticus” DNA synthesis is measured in the context of host tissue, it is not possible to conclude whether test conditions affect the pathogen directly or indirectly via altered host physiology. Moreover, “Ca. L. asiaticus” may benefit from the ability of another microbe to utilize glucose in the production of one or more secreted metabolite(s) subsequently acquired and used by “Ca. L. asiaticus”. Regardless, this study establishes a method for controlled activation of “Ca. L. asiaticus” DNA replication within natural tissue.

While the invention has been described in terms of its example embodiments, those skilled in the art will recognize that the invention can be practiced with modification within the spirit and scope of the appended claims. Accordingly, the present invention should not be limited to the embodiments as described above, but should further include all modifications and equivalents thereof with the spirit and scope of the description provided herein.

What is claimed is:

1. A bacterial growth medium, comprising:
a composition, wherein the composition further comprises:
a plurality of nutrients and
a Candidatus Liberibacter inoculum,
wherein the composition is configured as a solidified media with an adjusted neutral pH.

2. (canceled)

3. The bacterial growth medium of claim 1, wherein the plurality of nutrients comprises a first plurality of nutrients and a second plurality of nutrients.

4. The bacterial growth medium of claim 3, wherein the second plurality of nutrients comprises at least one of trace minerals, salts, and vitamins to enhance the composition.

5. The bacterial growth medium of claim 1, wherein the plurality of nutrients comprises least one of nutrient selected from the group consisting of alpha-ketoglutaric acid, ACE buffer, potassium hydroxide, phosphate buffer, deionized water, and any combination thereof.

6. The bacterial growth medium of claim 1, wherein the plurality of nutrients comprises one or more trace minerals, wherein the trace minerals comprise at least one trace mineral selected from the group consisting of nitrilotriacetic acid, magnesium chloride, iron sulfate, cobalt chloride, zinc chloride, copper sulfate, potash alum, boric acid, sodium molybdate, nickel chloride, sodium tungstate, sodium selenite, and any combination thereof.

7. (canceled)

8. The bacterial growth medium of claim 1, wherein the plurality of nutrients comprises one or more salts, wherein the salts comprise at least one salt selected from the group consisting of potassium chloride, ammonium chloride, sodium hydrogen phosphate, calcium chloride, magnesium sulfate, and any combination thereof.

9. (canceled)

10. The bacterial growth medium of claim 1, wherein the plurality of nutrients comprises one or more vitamins, wherein the vitamins comprise at least one vitamin selected from the group consisting of biotin, folic acid, pyridoxine hydrochloride, riboflavin, thiamine hydrochloride, nicotinic acid, DL-calcium pantothenate, vitamin B12, p-aminobenzoic acid, thiocidic acid, and any combination thereof.

11. (canceled)

12. The bacterial growth medium of claim 1, wherein the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof.

13. (canceled)

14. (canceled)

15. A method of growing a bacterium, said method comprising the steps of:
i) inoculating a composition using a Candidatus Liberibacter bacteria; and
ii) physiochemically adjusting the composition to comprise a pH between 7 and 12 and an oxygen tension of less than about 30% of air of the composition.

16. The method of claim 15, wherein the method further comprises the step of combining a plurality of nutrients with the composition.

17. The method of claim 15, wherein the method further comprises the step of combining a Candidatus Liberibacter inoculum with the composition.

18. The method of claim 17, wherein the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof.

19. (canceled)

20. The method of claim 15, wherein the composition is configured as a solidified media with an adjusted neutral pH.

21. The method of claim 15, wherein the Candidatus Liberibacter bacteria is derived from at least one bacteria selected from the group consisting of a leaf and one or more infected psyllids.

22. A host-free microbial culture, comprising:
a composition, wherein the composition further comprises:
a Candidatus Liberibacter inoculum;
a Bacillus species; and
one or more antibiotics.

23. The host-free microbial culture of claim 22, wherein the one or more antibiotics are not effective to inhibit growth of the Candidatus Liberibacter inoculum.

24. (canceled)

25. (canceled)

26. (canceled)

27. The host-free microbial culture of claim 22, wherein the one or more antibiotics comprises at least one antibiotic selected from the group consisting of a vancomycin antibiotic, a streptomycin antibiotic, and a polymyxin antibiotic.

28. The host-free microbial culture of claim 27, wherein the one or more antibiotics reduces growth abundance of the Candidatus Liberibacter inoculum.

29. The host-free microbial culture of claim 22, wherein growth of the Candidatus Liberibacter in the culture is inversely correlated with the abundance of the Bacillus species in the culture.

30. The host-free microbial culture of claim 22, wherein the Candidatus Liberibacter inoculum is at least one bacterium selected from the group consisting of Candidatus Liberibacter asiaticus, Candidatus Liberibacter americanus, Candidatus Liberibacter africanus, Candidatus Liberibacter solanacearum, and any combination thereof.

31.-51. (canceled)
